(19) United States
(12) Patent Application Publication
(10) Pub. No.: US 2010/0286290 A1
(43) Pub. Date: Nov. 11, 2010

(54) ENZYME ACTIVITY ASSAY USING ROLLING CIRCLE AMPLIFICATION

(76) Inventors: Jakob Schwalbe Lohmann, Ry (DK); Magnus Stougaard, Aarhus C (DK); Jørn Erland Koch, Ry (DK)

Correspondence Address: WEINGARTEN, SCHURGIN, GAGNEBIN & LEBOVICI LLP, TEN POST OFFICE SQUARE, BOSTON, MA, 02109, US

(21) Appl. No.: 12/602,806

(22) PCT Filed: Jun. 3, 2008
(86) PCT No.: PCT/DK08/50132

§ 371 Date: Apr. 20, 2010

(30) Foreign Application Priority Data
Jun. 4, 2007 (DK) PA 2007 00818
Jan. 16, 2008 (DK) PA 2008 00061

(51) Int. Cl. A61K 47/00; C12Q 1/68; A61P 35/00

(52) U.S. Cl. 514/789; 435/6

(57) ABSTRACT

The present invention relates to an enzyme activity assay using rolling circle amplification for verifying that a sample contains the enzyme activity in question. Thus, the present invention pertains to a method for determining the presence or absence of one or more enzyme activities involved in circularising a non-circular oligonucleotide probe in a biological sample. Furthermore, the present invention concerns liquid compositions comprising one or more oligonucleotide probes. Within the scope of the present invention is also a composition comprising a liquid composition and a tissue sample, and solid support of one or more oligonucleotides of the present invention. Disclosed is also a microfluidic device with one or more compartments for performing rolling circle amplification events, and a method for correlating one or more rolling circle amplification events. Methods for testing the efficacy of a drug, for diagnosing or prognosing a disease, for treating a disease, or for treating prophylactically a disease is furthermore disclosed.

All patent and non-patent references cited in this application are hereby incorporated by reference in their entirety.

### FIELD OF INVENTION

The present invention relates to an enzyme activity assay using rolling circle amplification for verifying that a sample contains the enzyme activity in question.

### BACKGROUND OF INVENTION

At present, a determination of the biological processes which take place in a single cell requires laborious and time consuming investigations at multiple levels.

DNA can be evaluated using different in situ hybridization techniques, such as FISH (Levsky & Singer, 2003), PRINS (Koch et al., 1989) or target primed amplification of padlock probes (Larsson et al., 2004). RNA detection is mainly performed using FISH techniques.

Detection of proteins are routinely performed using antibodies, but new techniques are emerging, e.g. proximity ligation, where oligonucleotide tagged antibodies are used for the detection interacting proteins by either PCR or rolling circle replication (Fredriksson et al., 2002; Soderberg et al., 2006). In particular, detection of DNA modifying enzymes is dominated by techniques using radioactively labeled oligonucleotides, which are practical for monitoring different cleavage and ligation reactions in solution (Lisby et al., 2001; Friedrich-Heineken & Hubscher, 2004), but also inconvenient because of the radioactive labeling. Another way of measuring DNA cleavage and ligation events is by using the Comet assay (also called single-cell gel electrophoresis). In this system cells are embedded in agarose and lysed. Subsequently the nucleoids are electrophorized and the migration of the DNA in the gel-matrix is used as a measure of how much damage is present in the DNA (reviewed in (Collins, 2004)). By exposing the DNA to either damage causing agents (e.g. UV-light, chemicals, and nucleases) and/or damage repairing agents (e.g. cell extracts and specific repair enzymes) prior to electrophoresis, information on different repair events can be monitored at an overall level.

All of the above techniques measure only the presence of bio-molecules, providing only an indirect determination of the activity of a protein within a given cell of a tissue sample.

Gene amplifications do often not correlate quantitatively with a higher expression level of the gene in question, e.g. because the genes may also be over-expressed in the absence of gene amplification (Mueller et al., 2004). Similarly, RNA levels do not necessarily correlate with the corresponding amount of protein produced. Furthermore, no satisfactory correlation can be established between protein production and corresponding protein activity levels.

A further complication is that many genes are alternatively spliced and that give rise to different proteins having different activities. Additionally, many proteins are regulated by interaction with other proteins and/or by posttranslational modifications (e.g. phosphorylation, glycosylation, methylation, etc.). Accordingly, a misleading impression of the activities of various proteins expressed in a tissue sample will often be generated using state-of-the-art methods (Henneke et al., 2003).

Rolling circle replication (RCR) of small circular oligonucleotides was first described by Fire et al., who performed RCR of a circular single stranded oligonucleotide of only 34 nucleotides (Fire & Xu, 1995). This observation has been converted into techniques detecting single nucleotide differentiation on DNA in situ by the use of padlock probes (Nilsson et al., 1994; Larsson et al., 2004), detection of RNA (Stougaard et al., submitted) and the detection of proteins in solution and in situ using proximity ligation (Fredriksson et al., 2002; Soderberg et al., 2006).

### DISCLOSURE OF THE INVENTION

The present invention is directed to assays for the detection in a biological sample of enzyme activities, such as DNA modifying enzyme activities, such as nucleases and toposiomerases. The present invention makes it possible to design assays combining protein activity detection with RCR through the conversion of linear oligonucleotides into circular ones.

An oligonucleotide probe comprising a specific, unprocessed substrate moiety can be processed by a particular enzyme, or a particular class of enzymes, thereby generating a template for rolling circle amplification. Detection of the rolling circle amplification product serves as an indication for the presence in a biological sample of the enzyme activity in question. The assays of the present invention are both quantitative and qualitative, thereby enabling a more sensitive analysis of not only the presence of an enzyme, but also of the activity associated with the enzyme in question when the enzyme is present in a biological sample, such as a tissue sample and/or a body fluid sample.

More particularly, the present invention exploits unique features associated with a single stranded circular DNA molecule according to the present invention. I) The presence of a primer allows a polymerase to synthesize a long, single stranded product containing tandem repeats complementary to the circular molecule. II) The circular molecule is resistant to exonucleases. III) Parts of the circular molecule can be used as a unique label, allowing identification and multiplexing of the reaction (Larsson et al., 2004).

Although topoisomerases and certain nucleases are disclosed herein below in some detail, the invention is not limited to the detection of such enzymes in a biological sample.

### Topoisomerases

One class of enzymes of interest for the present invention is topoisomerases. Topoisomerases are a diverse group of enzymes relaxing DNA during replication and transcription (Champoux, 2001). Topoisomerase I is a monomeric enzyme which cleaves one strand in the DNA, allowing relaxation of the DNA (Champoux, 2001).

The double helical structure of DNA presents a challenge in terms of accessibility, organization and segregation of the genome inside cells. By opening transient breaks in the DNA backbone DNA topoisomerases assist in solving helical winding and tangling of DNA during its replication, transcription, recombination, condensation and segregation. These essential cellular enzymes have been found in organisms ranging from viruses to human and are generally divided into two main groups, type I and type II topoisomerases.

Type I topoisomerases introduce transient single stranded breaks in the DNA and are able to perform DNA relaxation in an ATP-independent manner. Type I topoisomerases are further divided in the subfamilies IA and IB. Type IA topoisomerases are Mg²⁺ dependent, require a partially single-stranded substrate and only relax negatively supercoiled DNA (Kirkegaard and Wang, 1985). Cleavage of the DNA substrate results in a cleavage-complex where the topoisomerase is covalently attached to the 5′ end of the DNA. Examples of this group are human topoisomerase IIIα and IIIβ. Type IB topoisomerases differ from IA enzymes by not requiring a partially single-stranded substrate and by relaxing both negatively and positively supercoiled DNA even in the absence of a metallic cofactor, although Mg²⁺ and Ca²⁺ stimulate the relaxation activity (Goto et al., 1984; Liu and Miller, 1981). Furthermore, they cleave the DNA by forming a covalent bond to the 3′ end of the DNA (Slesarev et al., 1994; Slesarev et al., 1993). An example of this group is human topoisomerase I.

In cervical tumors a comparison of topo I protein levels and enzymatic activity revealed that topoisomerase I activity did not correlate with topo I protein levels. A similar lack of a correlation between topo I protein levels and topo I activity has been reported for malignant ovarian tumors [31, 33]. In addition, cellular sensitivity to SN-38 was positively correlated with topo I activity but not to topo I mRNA expression in human colon cancer cell lines. Sensitivity to SN-38 in a panel of human lung cancer cells also did not correlate with topo I protein levels [34]. In contrast, a relationship between topo I protein levels and catalytic activity in colon and prostate tumors [28] and a trend of increased topo I protein with enzymatic activity in several tumor types [35] have been reported. It is unclear why conflicting results have been obtained for the relationship between topo I protein levels and catalytic activity but one explanation is differential posttranslational modification of topo I protein. Topo I catalytic activity is increased by serine phosphorylation mediated by casein kinase type II [36] and protein kinase C [37], whereas topo I enzymatic activity is inhibited by poly(ADP-ribosylation) [38]. The catalytic activity and stability of topo I can also be increased by association with the tumor suppressor protein p53 [39]. It is tempting to speculate that the posttranslational modifications, and thus activity, of topoisomerase I may vary with tumor type or disease stage. (Elevated Topoisomerase I Activity in Cervical Cancer as a Target for Chemoradiation Therapy, Gynecologic Oncology, Volume 79, Issue 2, November 2000, Pages 272-280).

Topoisomerase I can also recognize artificial DNA substrates. A preferred substrate for topoisomerase I has been identified (Andersen et al., 1985). By dividing the top strand of the substrate into two segments and providing it with a three nucleotide 5′-overhang (flap structure), a molecular structure is generated where topoisomerase I is able to cleave three nucleotides off from the 3′-end. Following cleavage the three 3′-end nucleotides diffuse away allowing ligation of the new 3′-end to the 5′-end, Thus, by positioning an optimized recognition sequence in a self-templating probe containing a flap structure topoisomerase I is able to circularize the probe This flap should be complementary with other parts of the probe such as maximum 25% complementary, such as at least 25% complementary, or such as at least 75% complementary. The length of the flap structure is e.g. such as 1-20 nucleotides, such as e.g. 1-10 nucleotides, such as e.g. 1-5 nucleotides, such as e.g. 4 nucleotides, such as e.g. 3 nucleotides, or such as e.g. 2 nucleotides long, or such as e.g. 1 nucleotide. Furthermore, the probe specificity can be increased by positioning a modification at the 3′-end, such as, but not limited to, PO₃, CH₃, a C-linker, NH₃, CH₂CH₃, or H. Such modification will prevent a ligase from circularizing the probe. To prevent a nuclease from digesting the 5′-overhang the overhang can be blocked by introducing one or more artificial nucleobases, backbone units, internucleoside linkers or sugar moieties in the overhang. could be modified to inhibit exonuclease activity.

In a preferred embodiment both the 3′-end is blocked and the 5′-overhang is blocked by introducing one or more artificial nucleobases, backbone units, internucleoside linkers or sugar moieties in the overhang.

Topoisomerase I is able to recognize the double stranded region of the probe cleaving of three nucleotides from the 3′-end and ligate the new 3′-end to the 5′-end, thereby circularizing the probe.

As described earlier, the probes can be incubated with a sample using buffer conditions enabling protein activity of one or more enzymes. In the case of topoisomerase I, metallic cofactors can potentially be omitted thereby inhibiting many enzymes, but not topoisomerase I which, as described earlier, maintains activity in the absence of metallic cofactors.

In a similar approach the described method can be used to screen for drugs inhibiting the reaction. This can be done by supplementing the sample incubation reaction with one or more drugs. The difference in number of circularized probes between a sample with drugs and without drug, may tell if the drugs have inhibited the reaction.

Type II topoisomerases generate transient double stranded breaks in the DNA helix and transport a second DNA helix through the gap. They all require Mg²⁺ and ATP and perform their catalytic action as either homodimers or heterotetramers. The discovery of an atypical type II topoisomerase, topoisomerase VI from S. shibatae, resulted in a division of the type II topoisomerases into two subfamilies, type IIA and type IIB (Bergerat et al., 1997). All eukaryotic type IIA topoisomerases are homodimeric, whereas the prokaryotic counterparts are heterotetrameric.

Type IIA topoisomerases leave a four base pair overhang during cleavage of the DNA and bind covalently to the 5′ end of the DNA (Liu et al., 1983; Sander and Hsieh, 1983). Examples of the type IIA subfamily are human topoisomerase IIα and IIβ.

The type IIB, topoisomerase VI from S. shibatae, differs significantly in sequence from the type IIA topoisomerases and is a heterotetramer like most of the other prokaryotic type II topoisomerases. Cleavage of the DNA results in a two-nucleotide 5′-overhang and a covalent attachment of the topoisomerase to the 5′ end of the DNA (Buhler et al., 2001).

An increased focus on this broad group of enzymes since the discovery of human topoisomerase I and human topoisomerase IIα as the sole target for several chemotherapeuticals.

### Nucleases

Another class of DNA modifying enzymes of interest for the present invention is nucleases involved in DNA repair. One of these enzymes, Fen1 (Flap Endonuclease 1), recognizes 5′-flap structures and cleaves the overhang at the base of the flap, thereby preparing the DNA for ligation (Harrington & Lieber, 1994). These types of structures are believed to be created in cells through Okazaki fragment elongation during replication (Liu et al., 2004) and during long-patch base excision-repair (Klungland & Lindahl, 1997). The importance of Fen1 is underlined by the observation that Fen1 (−/−) mice do not survive past the blastocyst stage (Larsen et al., 2003) and mice which are heterozygous in both the Fen1 gene and the adenomatous polyposis coli (Apc) gene have an increased number of adenocarcinomas (Kucherlapati et al., 2002).

### Flap Endonuclease 1 (Fen1)

Fen1 was first described as a structure specific endonuclease, which cleaves 5′-flap structures at the base of the flap (Harrington & Lieber, 1994). Fen1 is believed to track along the 5′-overhang starting from the 5′-end and when the base of the flap is reached Fen1 cleaves off the overhang (see FIG. 8).

Furthermore, a free 5′-end is an absolute requirement since blockage of the 5′-end by e.g. streptavidine inhibits Fen1 binding (Murante et al., 1995). Fen1 is believed to be involved in numerous biological processes in human cells, which is also underlined by the observation that Fen1 (−/−) mouse blastocysts cannot enter S phase to carry out normal DNA synthesis leading to cell cycle arrest and cell death (Larsen et al., 2003) and mice which are heterozygous in both the Fen1 gene and the adenomatous polyposis coli (Apc) gene have an increased number of adenocarcinomas (Kucherlapati et al., 2002).

5′-flap structures are believed to be created in human cells during several vital processes such as DNA replication and DNA repair. During DNA replication an essential step in lagging strand DNA synthesis is removal of the RNA primer and ligation of Okazaki fragments. Elongation from an Okazaki fragment results in a collision between the polymerase and the RNA part of the next Okazaki fragment, leading to strand displacement which creates 5′-flap structures. Fen1 is able to remove the flap thereby preparing the nick for ligation (Murante et al., 1996; Qiu et al., 1999). Fen1 is also involved in DNA repair. Cells are constantly exposed to DNA damage caused by exposure to either endogenous reactive metabolites or exogenous damaging agents which can e.g. oxidize or alkylate DNA (Lindahl, 1993). One of the major products resulting from oxidation is 8-oxo-2′-deoxyguanosine (8-oxo-G), which is estimated to be generated 10⁴ to 10⁵ times per cell per day (Ames et al., 1993). What makes 8-oxo-G dangerous, if the base is not repaired, is that it can base pair with A, which can lead to G:C to A:T conversions following replication. Other major base damages are e.g. C to U transitions, abasic sites, methylations and formamidopyriminer (Lindahl, 1993). The main pathway for correcting damaged bases is the DNA base excision repair (BER) pathway. Damaged bases are recognized by a DNA glycosylase which catalyzes the hydrolysis of the glycosidic bond between the modified base and the sugar moiety to the release the base and generate an abasic site (apurinic/apyrimidic (AP) site). Numerous DNA glycosylases exist with different or overlapping substrate specificities Ode & Kotera, 2004; Dizdaroglu, 2005). Subsequently to base removal the AP endonuclease 1 (APE1) cleaves the backbone at the 5′-end of the AP site. From this point two different mechanism are believed to be able to reconstitute the DNA strand, either the short patch BER(SP-BER) or the long patch BER (LP-BER) (Liu et al., 2004) (see FIG. 9).

In SP-BER, DNA polymerase 13 removes the abasic sugar residue and fills the gap, thereby preparing the DNA for ligation. However if the abasic sugar residue is reduced or oxidized it cannot be removed and is therefore subjected to LP-BER. In this case a polymerase extends from the nick and displaces the downstream nucleotides thereby creating a 5′-flap which can be removed by Fen1 followed by ligation Ode & Kotera, 2004).

Although Fen1 is detectable in situ using antibodies (Warbrick et al., 1998) it does not necessarily tell anything about the activity of the enzyme, since the activity is regulated by interactions with other enzymes, phosphorylations and acetylations (Li et al., 1995; Hasan et al., 2001; Henneke et al., 2003; Zheng et al., 2007). Therefore, assays enabling detection of Fen1 activity from small amounts of cells or tissue would be a strong tool to further elucidate Fen1 activities. Furthermore, if an in situ assay for Fen1 activity can be optimized, it may be useful as a cell cycle marker, since it is cell cycle regulated, in part through phosphorylations (Henneke et al., 2003) which is difficult to detect with antibodies.

In general, by having a self-templating probe comprising a 5′-overhang or 3′-overhang the activity of a broad spectrum of nucleases can be detected, such as 3′-exonucleases, 5′-exonucleases, 3′-endonucleases and 5′-endonucleases, depending on the type of overhang. If you want to make the probe more specific one or more nucleobases, backbone units, internucleoside linkers or sugar moieties in the overhang could be modified to inhibit exonuclease activity.

Blocking for 5′-exonuclease Activity:

By positioning one or more modified nucleobases, backbone units, internucleoside linkers or sugar moieties in the 5′-overhang, 5′-exonucleolytically degradation could be inhibited, whereas the 5′-endonuclease activity would likely not be blocked of enzymes such as DNA2P, exol and Fen1. Furthermore, by positioning a cap on the 5′-end inhibiting ligation, unspecific ligation could be inhibited minimizing false positive signals. This ligase activity could, besides be caused by ligase, also be caused by e.g. topoisomerase I. Following endonucleolytically cleavage by e.g. Fen1, a ligase would be able to seal the gap, since the cap has been removed. Preferably the ligase is present in the sample, meaning that the activity of more than one enzyme is detected.

Alternatively the ligase is supplemented to the reaction mixture or added in a subsequent reaction.

Thus, in one embodiment, the 5′-end is capped with a modification selected from the group, but not limited to, PO₃, CH₃, a C-linker, NH₃, CH₂CH₃, biotin or H.

Thus, in a second embodiment, the 5′-overhang comprises one or more modified nucleobases, backbone units, internucleoside linkers or sugar moieties inhibiting exonucleolytically degradation.

Blocking for 3′-exonuclease Activity:

By positioning one or more modified nucleobases, backbone units, internucleoside linkers or sugar moieties in the 3′-overhang, 3′-exonucleolytically degradation could be inhibited, whereas 3′-endonuclease activity would likely not be blocked, Furthermore, by positioning a cap on the 3′-end inhibiting ligation, unspecific ligation could inhibited. This ligase activity could, besides be caused by ligase, also be caused by e.g. a topoisomerase II. Following endonucleolytic cleavage, a ligase would be able to seal the gap, since the cap has been removed.

Thus, in one embodiment, the 3′-end is capped with a modification selected from the group, but not limited to, PO₃, CH₃, a C-linker, NH₃, CH₂CH₃, biotin or H.

Thus, in a second embodiment, the 3′-overhang comprises one or more modified nucleobases, backbone units, internucleoside linkers or sugar moieties inhibiting exonucleolytic degradation.

The number of modifications should be, but not limited to, such as 1-10 modifications, such as 3-5 modifications, or such as 3 modifications.

The flap should be complementary with other parts of the probe such as maximum 25% complementary, such as at least 25% complementary, or such as at least 75% complementary. The length of the flap structure is e.g. such as 1-100 nucleotides, such as e.g. 1-80 nucleotides, such as e.g. 1-60 nucleotides, such as e.g. 1-30 nucleotides, such as e.g. 1-20 nucleotides, or such as e.g. 1-10 nucleotides long, or such as e.g. 3-10 nucleotides.

In a further aspect of the present invention, the effects of macromolecules or drugs affecting the activity of DNA modifying enzymes in a biological sample can be assayed by means of the present invention.

In particular, the present invention provides methods for the direct detection of one or more enzyme activities involved in an enzymatic pathway in a cell or a tissue sample being analyzed for the presence or absence of said one or more enzyme activities. In particular, the present invention makes it possible to directly detect if enzyme activities, such as topoisomerase activities, such as topoisomerase I activities, and nuclease activities, such as flap endonuclease activities, for example Fen1 activities, are present in a biological cell. The direct detection is made possible by introducing into the cell(s) in question a probe structure which is not a substrate for RCR and which can only be amplified by RCR if the probe structure is modified by an enzyme specifically able to modify the probe structure, thereby converting the probe structure into a substrate for subsequent amplification by RCR.

The methods of the present invention can employ any suitable probe structure, such as small, linear self-templating DNA probes with flap structures combined with RCR and fluorescent detection. The methods of the present invention are well suited for i) detection of enzyme activities in gels, ii) detection of enzyme activities in solid support assays, using either purified enzymes or cell extracts, and iii) for enzyme detection directly in single cells attached to a surface, or in tissue preparations.

A solid support associated with the oligonucleotide probes and circular templates according to the invention is also disclosed. This allows in situ amplification and detection of the amplification events.

Also disclosed is a microfluidic device which can be used for diverting samples comprising tissue and/or body fluid samples to and from a reaction chamber of the solid support.

Preferred embodiments of the present invention are disclosed herein below in more detail.

### Method for Determining Enzyme Activity Involved in Oligonucleotide Probe Circularization

In one aspect of the present invention there is provided a method for determining in a biological sample either a) the presence of one or more enzyme activities involved in circularising a non-circular oligonucleotide probe, or b) the absence of at least one such enzyme activity in said biological sample, said method comprising the steps of
i) providing a biological sample to be analysed for the presence or absence of at least one enzyme activity,
ii) providing an oligonucleotide probe comprising an unprocessed substrate moiety capable of being processed by at least one of said one or more enzymes,
   wherein said oligonucleotide probe comprises a single strand of contiguous nucleotides or a plurality of single strands of contiguous nucleotides capable of hybridisation to each other,
   wherein said oligonucleotide probe comprising an unprocessed substrate moiety cannot be amplified by rolling circle replication in the absence of said processing,
iii) contacting the biological sample with the oligonucleotide probe under conditions allowing said one or more enzymes, if present in said biological sample, to act on the substrate moiety,
   wherein said action results in the processing of the substrate moiety and the formation of a circular, oligonucleotide template capable of being amplified by rolling circle replication,
iv) amplifying the circular oligonucleotide template, when such a template is formed in step iii), by using a polymerase capable of performing multiple rounds of rolling circle replication of said circular oligonucleotide template, optionally by initially contacting said circular oligonucleotide template with a suitable primer, and generating a rolling circle amplification product comprising multiple copies of the circular oligonucleotide template, or
v) generating no rolling circle amplification product when no circular oligonucleotide template is formed in step iii) as a result of said one or more enzyme activities not being present in said biological sample,
   wherein steps iv) and v) are mutually exclusive,
   wherein said amplification product is indicative of the presence in said biological sample of said one or more enzyme activities involved in circularising a non-circular oligonucleotide probe,
   and wherein no amplification product is formed in the absence of at least one such enzyme activity in said biological sample.

The oligonucleotide probe can be designed in various ways depending on the purpose of the probe—as primarily defined by the unprocessed substrate moiety. The probe design makes it possible for the methods of the present invention to exploit an enzymatic circularization of oligonucleotide probes which can subsequently be detected by RCR (rolling circle replication), which amplification allows for single molecule detection.

The general design of certain preferred probes is illustrated in FIG. 1. Each probe contains a detection sequence, used to identify the probe following RCR, a primer recognition sequence, and a unique double stranded region which is optimized to bind the enzymes of choice (FIG. 1, A-C). Thus, a molecule which has been circularized by a specific enzyme can be amplified by RCR and visualized in the microscope. The Fen1 probe sequence was derived from the substrate T/DN2/UP3 described by Friedrich-Heineken E and Hubscher U (Friedrich-Heineken & Hubscher, 2004). Fen1 recognizes 5′-flaps and cleaves them endo-nucleolytically at the base of the flap, the strand-break can then be sealed by a ligase, leading to circularization of the probe. (FIG. 1A). To minimize non-specific ligation, the original 5′-end can be blocked with a biotin, which neither interfere with Fen1 binding nor cleavage (Murante et al., 1995). The Topo I probe sequence was derived from the substrate described by Andersen A H et al. (Andersen et al., 1985). Topoisomerase I recognizes the preferred double stranded sequence and cleaves the probe three nucleotides away from the 3′-end. The three far most nucleotides are removed by diffusion (Lisby et al., 2001). Following cleavage the 5′-flap can be ligated to the new 3′-end, thereby resulting in circularization of the probe (FIG. 1B). A probe for analysing gap repair is illustrated in FIG. 1C.

To minimize unspecific circularization events, the original 3′-end was blocked by a 3′-amin, which did not seem to interfere with the topoisomerase I catalysis. Following enzymatic circularization of the probes, they are turned into substrates for RCR (FIG. 1D). The primer for RCR can either be free in solution or coupled to a surface. The rolling circle product (RCP) could be detected by hybridizing a labeled oligonucleotide to the RCP followed by microscope analysis (FIG. 1E).

By analogy to the above disclose, reference is made to FIG. 2 illustrating an alternative probe design for the same enzyme activities.

Both types of assays can initially be tested in solution for cleavage and ligation performance and the reaction products analyzed by PAGE. RCR was performed in a solid support format with covalently coupled primers. Finally, the probes were tested on cells grown on teflon-printed diagnostic well-slides and on anonymous breast cancer tissue.

Preferred enzyme activities include, but are not limited to, topoisomerases and flap endonucleases. The methods of the present invention can be used for the detection of several types of enzymes and enzymatic pathways, such as, topoisomerases (such, as but not limited to, topoisomerase IA, topoisomerase IB, topoisomerase IIα, topoisomerase 116, topoisomerase III, and vaccinia virus topoisomerase I), resolvases/invertases and recombinases (such, as but not limited to, Flp recombinase, Cre recombinases, lambda Int integrase, HP1 integrase XerC recombinases, XerD recombinases).

The methods of the present invention can be multiplexed using a different fluorescent color code for each probe.

One important feature of the present invention is the design of the oligonucleotide probes used. The oligonucleotide probes can be circularized by the action of one or more enzyme activities present in a biological sample. The minimum requirements for an oligonucleotide probe according to the present invention are a detection sequence and a primer sequence, the rest of the probe can be designed according to the enzyme assay of choice.

Signals can also be obtained from cell and tissue preparations, which could be of great potential in evaluating which repair enzymes are active inside a cell, e.g. in cancer.

The probes of the invention are comprising one or more individual nucleic acid sequences. The probe can comprise any sequence of the natural nucleotides G, C, A, T, I, U, or any artificial nucleotides e.g., but not limited to, iso-dCTP, iso-dGTP, or a mixture thereof. The one or more individual nucleic acid sequences of the probes of the invention have a linear length of 20-200 nucleotides. Thus, in one aspect, the invention relates to a method, wherein the one or more probes have a length of 20-300 nucleotides, such as e.g. 20-150 nucleotides, or such as e.g. 20-100 nucleotides, or such as e.g. 20-80 nucleotides, or such as e.g. 20-60 nucleotides, or such as e.g. 20-40 nucleotides, or such as e.g. 20-30 nucleotides. The probes can be synthesized by standard chemical methods (e.g. beta-cyanoethyl phosphoramidite chemistry).

The probes possess several characteristics: 1) the probe comprises one or more complementary sequences, enabling the probe to hybridize to itself. 2) the probe comprises one or more loop structures connecting complementary sequences. 3) The probe possesses binding or interaction sites for one or more enzymes (so-called substrate moieties). 4) The probes are designed so that no part of the probe recognizes DNA or RNA sequences in the sample

### LOOP Structure of the Probes

The one or more loop structures of the probe aim to connect the ends of the two or more complementary sequences. The loop comprises 3-100 nucleotides, such as e.g. 3-80 nucleotides, or such as e.g. 3-60 nucleotides, or such as e.g. 3-40 nucleotides, or such as e.g. 3-30 nucleotides. The loop structures can serve one or more purposes. The loop can be used as primer recognition sequences for amplification reactions, e.g. for rolling circle DNA synthesis, or PCR. The loops can also serve as an identification element to identify specific probes. The loops also serve to connect one or more double stranded regions of the probe.

### Complementary Sequences of the Probes

The complementary sequences of the probe are positioned on each side of the one or more loop structures in the sequence of the probe. The complementary sequences comprise 3-100 nucleotides, such as e.g. 5-80 nucleotides, such as e.g. 10-60 nucleotides, such as e.g. 10-30 nucleotides, or such as e.g. 10-40 nucleotides. Preferably, the complementary sequences are 10-20 nucleotides long, such as e.g. 10-20 nucleotides, such as e.g. 12-20 nucleotides, such as e.g. 14-20 nucleotides, or such as e.g. 15-20 nucleotides.

The aim of the complementary sequences of the probe is to form a substrate or part of a substrate for one or more enzymes. Furthermore, the complementary sequences enable the probe to be circularized by self-templated hybridization of the complementary sequences in the probe.

The probe can be provided in several formats: In one format the probe, which consists of a single oligonucleotide, through self-templated hybridization is able to form a region which can be a substrate moiety for one or more enzymes. In another format the probe, which consists of more than one oligonucleotides, through hybridization, is able to form a region which can be a substrate moiety for one or more enzymes. It is to be understood that all of the following sections refers to both formats of the probe.

### The Sample

A sample can be provided in several formats such as, but not limited to, cells grown on a surface, cells in solution, cell extracts, tissue preparations or purified enzymes.

In a preferred embodiment of the invention, the invention relates to three aspects of enzymatic activities, which can be detected in a sample using probes having special unprocessed substrate moieties:
Nick/gap repair
Topoisomerase activity
Repair of overhangs and flap structures

Accordingly, in one embodiment there is provided an oligonucleotide probe further comprising one or more non-hybridised, single stranded portion(s) and one or more double stranded portion(s), each double stranded portion comprising complementary nucleotide strands. The one or more single stranded portion(s) of said oligonucleotide probe does not hybridise to a complementary nucleotide sequence, but the oligonucleotide probe also comprises at least one nucleotide sequence which is complementary to one or more of said single stranded portion(s) of said oligonucleotide probe.

The oligonucleotide probe can be in the form of a single oligonucleotide comprising a contiguous sequence of nucleotides, wherein at least some of said nucleotides are capable of forming a double stranded sequence comprising complementary nucleotide strands, or the oligonucleotide probe can comprise more than one single oligonucleotide, wherein each oligonucleotide of the probe comprises a single contiguous sequence of nucleotides, wherein at least some of said nucleotides of the different oligonucleotides of the probe are capable of hybridising to each other.

The probe can be a self-templating probe comprising at least two double stranded portions each comprising complementary nucleotide strands separated at the proximal ends by an unprocessed substrate moiety, and the at least two double stranded portions can comprise complementary nucleotide strands are each joined at the distal ends by a single stranded nucleotide forming a loop structure.

In one embodiment, the unprocessed substrate moiety can comprise a nick or a single stranded nucleotide region. The single stranded nucleotide region is adjoined at both ends to a double stranded nucleotide region and the single stranded nucleotide region can be a 5′ overhang nucleotide region or a 3′ overhang nucleotide region adjoined at one end to a double stranded nucleotide region of the oligonucleotide probe.

The single stranded nucleotide region preferably contains less than 20 nucleotides, such as less than 15 nucleotides, for example less than 10 nucleotides, such as less than 5 nucleotides, for example less than 3 nucleotides.

In one embodiment, the substrate moiety conversion is mediated specifically by a flap endonuclease activity present in said sample in combination with a ligase activity present in said sample and/or added to said sample. The flap endonuclease activity can be mediated by, for example, FEN1, DNA2P or EXO1. Below is provided a table with examples of the afore-mentioned enzymes and corresponding database accession numbers. The list is by no means to be regarded as exhaustive.

| Protein | Accession number |
|---|---|
| Flap endonuclease 1 (Fen1) | CAG38799 |
| Dna2P (DNA2) | CAI17238 |
| Exonuclease I (exo1) | Q9UQ84, NP_006018, |
| | NP_003677, NP_569082 |
| Topoisomerase I | CAA34500, |
| Topoisomerase II alpha | CAA09762 |
| Topoisomerase II beta | CAA09753 |
| FLP recombinases (FLP) | CAA71451 |
| Cre recombinases (Cre) | AAV28859 |

The substrate moiety conversion is in another embodiment mediated specifically by a topoisomerase activity present in said sample, such as a Topoisomerase I or a Topoisomerase II activity.

It is advantageous to use a set of probes which allows the skilled practitioner in the clinic to rapidly determine not only the kind of enzyme present in a biological sample, but also the level of activity exhibited by said enzyme. Accordingly, there is also provided a method wherein more than one type of unprocessed substrate moiety is employed. Accordingly, there is provided a method wherein the unprocessed substrate moiety is preferably selected from the group consisting of
i) unprocessed substrate moieties comprising or consisting of one or more nick(s) in one or more single strand(s) of a double stranded nucleotide sequence of said oligonucleotide probe, said one or more nick(s) forming one or more unprocessed substrate moieties of said oligonucleotide probe,
ii) unprocessed substrate moieties comprising or consisting of one or more single stranded nucleotide sequence(s) joined at one or both ends thereof by a double stranded nucleotide sequence, said single stranded sequence(s) creating one or more gap structure(s) forming one or more unprocessed substrate moieties of said oligonucleotide probe, and
iii) unprocessed substrate moieties comprising or consisting of one or more nick(s) or one or more gap(s), said gap(s) being in the form of a single stranded nucleotide sequence, said nick(s) or gap(s) being joined at one end thereof to a double stranded nucleotide sequence and at the other end thereof to at least one single stranded overhang joined to a double stranded nucleotide sequence of said oligonucleotide probe, wherein said nick(s) or gap(s) in combination with the at least one single stranded overhang forms one or more unprocessed substrate moieties of said oligonucleotide probe.

Unprocessed substrate moieties comprising nicks are disclosed in detail herein below. Probes comprising such unprocessed substrate entities preferably comprises an unprocessed substrate moiety which comprises or consists of one or more nick(s) in one or more single strand(s) of a double stranded nucleotide sequence of said oligonucleotide probe, said one or more nick(s) forming one or more unprocessed substrate moieties of said oligonucleotide probe. The one or more enzyme activities present in the biological sample comprises a ligase activity capable of ligating said nick of said oligonucleotide probe. Alternative, a ligase can be added to the sample as a control measurement or for other reasons when the ligase activity is not limiting for the assay to be conducted. The important issue in this respect is that a circular oligonucleotide template capable of being amplified by rolling circle amplification is generated by ligating said nick, said ligation being performed by at least one ligase activity present in said sample.

The generated, circular oligonucleotide template is subsequently amplified by rolling circle amplification and said amplification can be indicative of or evidence of the presence in said sample of at least one ligase activity—provided that the ligase activity is not added to the sample. However, nick ligation can be used as an “internal control” when one or more further enzyme activities are required in order to circularise the oligonucleotide probe—and when a ligase activity in the biological sample in itself is not of any interest to the assay in question.

The generated rolling circle amplification product can be detected by detecting a label covalently or non-covalently associated with said rolling circle amplification product, wherein said label is preferably fluorescently detectable, wherein said label is either a fluorescent molecule incorporated into the rolling circle amplification product, for example by being present in the primer used for probe amplification and generation of the rolling circle amplification product, or by being linked to a nucleotide incorporated into the rolling circle amplification product during the probe amplification process, or by being linked to a fluorescently labelled oligonucleotide hybridising to the rolling circle amplification product, or wherein said label is a molecule or a chemical group which can be detected by a fluorescently labelled molecule, such as an antibody.

Oligonucleotide probes comprising gaps are disclosed in more detail herein below. When such an oligonucleotide probe is employed, there is provided one or more probes each comprising one or more unprocessed substrate moieties which comprises or consists of one or more single stranded nucleotide sequence(s) joined at one or both ends by a double stranded nucleotide sequence, said single stranded sequence(s) creating one or more gap structure(s) forming one or more unprocessed substrate moieties of said oligonucleotide probe. A circular, oligonucleotide template capable of being amplified by rolling circle amplification can be generated through a) filling-in said gap by using the at least one enzyme activity present in said sample which is capable of performing a template directed nucleotide extension reaction, and b) ligating one or both of the end-positioned, filled-in nucleotides to the remaining, double stranded part of the oligonucleotide probe.

The circular, oligonucleotide template can be amplified by rolling circle amplification, said amplification being indicative of the presence in said sample of at least one enzyme activity capable of performing template directed nucleotide extension and/or nucleotide ligation. The rolling circle amplification product can be detected as disclosed herein above.

In one embodiment of the invention, the invention relates to detection of enzyme activity of enzymes or enzymatic pathways involved in gap repair such as, but not limited to, polymerases, kinases, ligases and accessory proteins.

It has been shown that the gap-filling DNA repair activity is markedly decreased in aging neuronal extracts and that this activity could be restored significantly by the addition of pure recombinant polymerase β and T4 DNA ligase. Thus, gap repair seems to be an important factor, which is changing with the aging of cells. Therefore, an assay monitoring the gap repair state of a cell or a sample could be an important molecular tool. (Reduced DNA gap repair in aging rat neuronal extracts and its restoration by DNA polymerase β and DNA-ligase, Journal of Neurochemistry, Volume 92 Issue 4 Page 818—February 2005). Gap repair is an intermediate step in several repair mechanisms, such as mismatch repair, loop repair, base excision repair and nucleotide excision repair.

A gap in the double stranded region can be repaired by a repair mechanism minimally involving a polymerase and a ligase. To simplify the reaction the probe can be 5′-phosphorylated in advance, leaving out the necessity for a kinase. The gap should have a length of such as e.g. 1-50 nucleotides, such as e.g. 1-40 nucleotides, such as e.g. 1-30 nucleotides, such as e.g. 1-20 nucleotides, such as e.g. 1-10 nucleotides, such as e.g. 1-5 nucleotides, such as e.g. 1-5 nucleotides, such as e.g. 4 nucleotides, such as e.g. 3 nucleotides, such as e.g. 2 nucleotides long, such as e.g. 1 nucleotides long, or such as e.g. 0 nucleotide. A length of 0 nucleotides corresponds to a nick, which would enable detection of ligase activity in the absence of polymerase activity.

Overhang sequences of the oligonucleotide probes according to the present invention are disclosed in more detail herein below. In one embodiment there is provided an oligonucleotide probe which comprises a substrate moiety which comprises or consists of one or more nick(s) and/or one or more gap(s), said gap(s) being in the form of a single stranded nucleotide sequence, said nick(s) or gap(s) being joined at one end to a double stranded nucleotide sequence and at the other end to at least one single stranded overhang joined to a double stranded nucleotide sequence of said oligonucleotide probe, wherein said nick(s) or gap(s) in combination with the at least one single stranded overhang forms one or more unprocessed substrate moieties of said oligonucleotide probe.

The one or more overhang(s) can be a 5′ overhang, said oligonucleotide probe further comprising at least one 3′ end and the 5′ overhang can be protected by a protection group preventing an exonuclease from digesting the 5′ overhang.

A circular, oligonucleotide template capable of being amplified by rolling circle amplification can be generated by a) endonucleolytic digestion of said 5′ overhang and b) ligation of the end of the nucleotide strand resulting from the endonucleolytic digestion to a nucleotide strand of the remaining part of the oligonucleotide probe. The circular, oligonucleotide template generated can be amplified by rolling circle amplification, said amplification being indicative of the presence in said sample of at least one endonuclease.

Also in this case can the rolling circle amplification product be detected by detecting a label covalently or non-covalently associated with said rolling circle amplification product, wherein said label is preferably fluorescently detectable, wherein said label is either a fluorescent molecule incorporated into the rolling circle amplification product, for example by being present in the primer used for probe amplification and generation of the rolling circle amplification product, or by being linked to a nucleotide incorporated into the rolling circle amplification product during the probe amplification process, or by being linked to a fluorescently labelled oligonucleotide hybridising to the rolling circle amplification product, or wherein said label is a molecule or a chemical group which can be detected by a fluorescently labelled molecule, such as an antibody.

5′ overhang sequences of oligonucleotide probes are disclosed below in more detail. The 5′ end of the 5′ overhang can comprise a protection group in the form of a phosphate group or different from a phosphate group, wherein said protection group prevents ligation of said 5′ overhang to a 3′ end of a strand of the remaining part of the oligonucleotide probe. The protection group different from a phosphate group can be selected from the group consisting of H, biotin, amin, and an optionally substituted C₁-C₆-linker.

One purpose of the protection group is to prevent a topoisomerase I activity present in the biological sample from processing the unprocessed substrate moiety of said oligonucleotide probe and generate a circular oligonucleotide template. Effectively preventing the activity of a topoisomerase allows a flap endonuclease activity present in said sample to process the unprocessed substrate moiety of said oligonucleotide probe, wherein said processing results in the formation of a 5′ end having a phosphate reactive group capable of being ligated with a 3′ end of a strand of the remaining part of the oligonucleotide probe, thereby generating a circular oligonucleotide template capable of being amplified by rolling circle amplification.

Accordingly, there is provided a method whereby a circular oligonucleotide template generated by the flap endonuclease activity and a ligase activity present in said sample is amplified by rolling circle amplification, thereby generating a rolling circle amplification product indicative of the presence in said sample of a flap endonuclease activity and a ligase activity.

It is also possible for the 3′ end of the oligonucleotide probe to comprise a protection group different from a hydroxy group, wherein said protection group prevents ligation of said 5′ overhang to the 3′ end of a strand of the remaining part of the oligonucleotide probe. The protection group different from a hydroxy group can be selected from the group consisting of H, biotin, amin, and an optionally substituted C₁-C₆-linker.

The protection of the 3′ end means that a flap endonuclease activity present in said biological sample cannot process the unprocessed substrate moiety of said oligonucleotide probe and provide an oligonucleotide which can be ligated by a ligase to generate a circular oligonucleotide template. This in turn enables a topoisomerase I activity present in said sample to process the unprocessed substrate moiety of said oligonucleotide probe, wherein said processing results in the formation of a 3′-phospho-tyrosine intermediate, in the form of a covalent DNA-protein intermediate, capable of being ligated with the HO-group of the 5′-end of the 5′-overhang of the oligonucleotide probe, wherein said ligation results in the formation of a circular oligonucleotide template capable of being amplified by rolling circle amplification.

The circular oligonucleotide template generated by the topoisomerase I activity present in said sample can subsequently be amplified by rolling circle amplification, thereby generating a rolling circle amplification product which is indicative of or evidence of the presence in said sample of a topoisomerase I activity.

The individual nucleotides of the 5′ overhang of the oligonucleotide probe is disclosed in more detail herein below. Each nucleotide of the 5′ overhang of the oligonucleotide probe comprises a nucleobase and a backbone unit, wherein the backbone unit comprises a sugar moiety and an internucleoside linker. The nucleobase of the nucleotides of the 5′ overhang can be selected from naturally occurring nucleobases and non-naturally occurring nucleobases. The backbone unit of neighbouring nucleobases can be selected from naturally occurring backbone units and non-naturally occurring backbone units. The sugar moiety of the backbone unit of neighbouring nucleobases can be selected from naturally occurring sugar moieties and non-naturally occurring sugar moieties. The internucleoside linker of the backbone unit of neighbouring nucleobases can be selected from naturally occurring internucleoside linkers and non-naturally occurring internucleoside linkers.

In one embodiment, the nucleobases of the 5′ overhang are selected independently from the group consisting of natural and non-natural purine heterocycles, natural and non-natural pyrimidine heterocycles, including heterocyclic, non-natural analogues and tautomers of said natural purine heterocycles and said natural pyrimidine heterocycles. Accordingly, the nucleobases of the 5′ overhang are selected, in one embodiment, independently from the group consisting of adenine, guanine, isoguanine, thymine, cytosine, isocytosine, pseudoisocytosine, uracil, inosine, purine, xanthine, diaminopurine, 8-oxo-N⁶-methyladenine, 7-deazaxanthine, 7-deazaguanine, N⁴,N⁴-ethanocytosin, N⁶,N⁶-ethano-2,6-diamino-purine, 5-methylcytosine, 5-(C³—C⁶)-alkynylcytosine, 5-fluorouracil, 5-bromouracil and 2-hydroxy-5-methyl-4-triazolopyridine.

The backbone units of the nucleotides of the 5′ overhang can be the same backbone units or different backbone units, wherein, preferably, the same or different backbone units of the nucleotides of the 5′ overhang are selected independently from the group consisting of

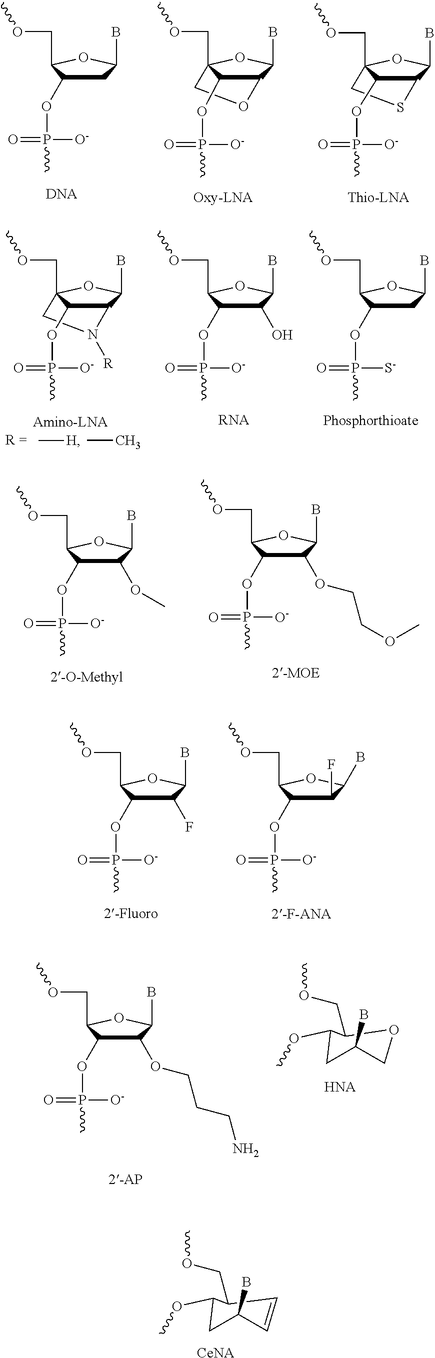

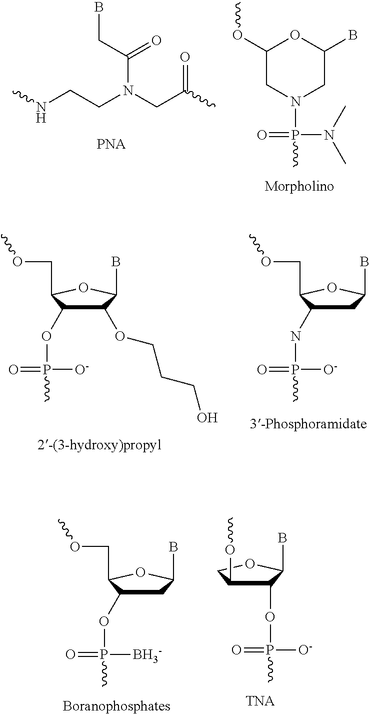

wherein B denotes a nucleobase.

The sugar moiety of the backbone unit of the nucleotides of the 5′ overhang preferably comprises or consists of a pentose, such as a pentose selected from the group consisting of ribose, 2′-deoxyribose, 2′-O-methyl-ribose, 2′-fluor-ribose, and 2′-4′-O-methylene-ribose (LNA). The nucleobase of the nucleotide is preferably attached to the 1′ position of the pentose.

The backbone units linking any two neighbouring nucleotides of the 5′ overhang can be the same or different backbone units. In one embodiment, at least some of the nucleotides of the 5′ overhang are linked by different backbone units. At least some of said different backbone units can be non-natural backbone units.

The internucleoside linkers linking any two neighbouring nucleotides of the 5′ overhang can be the same or different internucleoside linkers. In one embodiment, at least some of the nucleotides of the 5′ overhang are linked by different internucleoside linkers. At least some of said different internucleotide linkers are non-natural internucleotide linkers. Generally, the internucleoside linkers of the 5′ overhang can be selected from the group consisting of phosphodiester bonds, phosphorothioate bonds, methylphosphonate bonds, phosphoramidate bonds, phosphotriester bonds and phosphodithioate bonds.

The nucleotides of the 5′ overhang is in one embodiment selected from naturally occurring nucleosides of the DNA and RNA family connected through phosphodiester linkages and at least one non-natural nucleotide selected from the group consisting of nucleotides comprising a non-natural nucleobase and/or a non-natural backbone unit comprising a non-natural sugar moiety and/or a non-natural internucleoside linker.

In another embodiment, the 5′ overhang comprises naturally occurring nucleobases connected by naturally occurring backbone units, wherein said naturally occurring nucleobases and said naturally occurring backbone units do not prevent exonuclease degradation of said 5′ overhang. The 5′ overhang can further comprise non-naturally occurring nucleobases which do not prevent exonuclease degradation of said 5′ overhang, for example non-naturally occurring backbone units comprising sugar moieties and internucleoside linkers which do not prevent exonuclease degradation of said 5′ overhang, or sugar moieties are non-naturally occurring sugar moieties which do not prevent exonuclease degradation of said 5′ overhang, or non-naturally occurring internucleoside linkers which do not prevent exonuclease degradation of said 5′ overhang. In this embodiment, the one or more enzyme activities present in said sample comprises a 5′ to 3′ exonuclease activity capable of cleaving one or more, such as all of the internucleoside linkers connecting the nucleotides of the 5′ overhang and/or a ligase activity. The generated, circular oligonucleotide template capable of being amplified by rolling circle amplification is generated by ligating the oligonucleotide probe comprising a substrate moiety processed by 5′ to 3′ exonucleolytically digestion of the 5′ overhang, said ligation being performed by at least one ligase activity present in said sample or added to said sample. The generated, circular, oligonucleotide template can be amplified by rolling circle amplification, wherein said amplification is indicative of or evidence of the presence in said sample of at least one 5′ to 3′ exonuclease activity and at least one ligase activity. The rolling circle amplification product can be detected by detecting a label covalently or non-covalently associated with said rolling circle amplification product, wherein said label is preferably fluorescently detectable, wherein said label is either a fluorescent molecule incorporated into the rolling circle amplification product, for example by being present in the primer used for probe amplification and generation of the rolling circle amplification product, or by being linked to a nucleotide incorporated into the rolling circle amplification product during the probe amplification process, or by being linked to a fluorescently labelled oligonucleotide hybridising to the rolling circle amplification product, or wherein said label is a molecule or a chemical group which can be detected by a fluorescently labelled molecule, such as an antibody.

In another embodiment disclosed in more detail herein below, the 5′ overhang comprises non-naturally occurring nucleobases connected by naturally occurring backbone units and/or non-naturally occurring backbone units, said backbone units comprising a sugar moiety and an internucleoside linker, wherein said non-naturally occurring nucleobases and said non-naturally occurring backbone units, when present, prevent exonuclease degradation of said 5′ overhang. The non-naturally occurring nucleobases alone can prevent exonuclease degradation of said 5′ overhang, or the non-naturally occurring backbone units alone can prevent exonuclease degradation of said 5′ overhang, or the non-naturally occurring sugar moieties alone can prevent exonuclease degradation of said 5′ overhang, or the non-naturally occurring internucleoside linkers alone can prevent exonuclease degradation of said 5′ overhang. In this embodiment, a 5′ to 3′ exonuclease activity present in said sample cannot cleave the internucleoside linkers connecting the nucleotides of the 5′ overhang./JLN However, when said one or more enzyme activities present in said biological sample further comprises a flap endonuclease activity, the flap endonuclease is capable of cleaving the internucleoside linkers connecting the nucleotides of the 5′ overhang and/or a ligase activity. Accordingly, when a circular, oligonucleotide template capable of being amplified by rolling circle amplification is generated by ligating the oligonucleotide probe comprising a processed substrate moiety, wherein said substrate moiety processing comprises flap endonucleolytically cleaving at least one internucleoside linker of the 5′ overhang of the probe, thereby releasing the 5′ overhang from the remaining part of the oligonucleotide probe, and wherein the ligation is performed by at least one ligase activity present in said sample, the generated, circular, oligonucleotide template can be amplified by rolling circle amplification, wherein said amplification is indicative of or evidence of the presence in said sample of at least one flap endonuclease activity and at least one ligase activity.

The rolling circle amplification product can be detected by detecting a label covalently or non-covalently associated with said rolling circle amplification product, wherein said label is preferably fluorescently detectable, wherein said label is either a fluorescent molecule incorporated into the rolling circle amplification product, for example by being present in the primer used for probe amplification and generation of the rolling circle amplification product, or by being linked to a nucleotide incorporated into the rolling circle amplification product during the probe amplification process, or by being linked to a fluorescently labelled oligonucleotide hybridising to the rolling circle amplification product, or wherein said label is a molecule or a chemical group which can be detected by a fluorescently labelled molecule, such as an antibody.

### 3′ Overhang Sequences of Oligonucleotide Probes

There is also provide a method wherein one or more overhang(s) of the employed oligonucleotide probe is a 3′ overhang, wherein said oligonucleotide probe further comprising at least one 5′ end. Such methods are disclosed in more detail herein below. Generally, the physical features which have been disclosed herein above for methods employing 5″overhang sequences also apply to methods employing 3′ overhang sequences. However, the specificity of the methods employing 3′ overhang sequences is different from the specificity of the methods employing 5′ overhang sequences. This will also be clear from the below disclosure.

The 3′ overhang can be protected by a protection group preventing an exonuclease from digesting the 3′ overhang. Accordingly, when a circular, oligonucleotide template capable of being amplified by rolling circle amplification is generated by a) endonucleolytic digestion of said 3′ overhang and b) ligation of the end of the nucleotide strand resulting from the endonucleolytic digestion to a nucleotide strand of the remaining part of the oligonucleotide probe, such a circular, oligonucleotide template can be amplified by rolling circle amplification, wherein said amplification being indicative of the presence in said sample of at least one endonuclease. The rolling circle amplification product can be detected by detecting a label covalently or non-covalently associated with said rolling circle amplification product, wherein said label is preferably fluorescently detectable, wherein said label is either a fluorescent molecule incorporated into the rolling circle amplification product, for example by being present in the primer used for probe amplification and generation of the rolling circle amplification product, or by being linked to a nucleotide incorporated into the rolling circle amplification product during the probe amplification process, or by being linked to a fluorescently labelled oligonucleotide hybridising to the rolling circle amplification product, or wherein said label is a molecule or a chemical group which can be detected by a fluorescently labelled molecule, such as an antibody.

Using the above-cited method, and when a topoisomerase II activity is present in said sample, processing of the unprocessed substrate moiety of said oligonucleotide probe provides a circular oligonucleotide template, wherein said circular oligonucleotide template generated by the topoisomerase II activity present in said sample is subsequently amplified by rolling circle amplification, thereby generating a rolling circle amplification product which is indicative of or evidence of the presence in said sample of a topoisomerase II activity.

The rolling circle amplification product can be detected by detecting a label covalently or non-covalently associated with said rolling circle amplification product, wherein said label is preferably fluorescently detectable, wherein said label is either a fluorescent molecule incorporated into the rolling circle amplification product, for example by being present in the primer used for probe amplification and generation of the rolling circle amplification product, or by being linked to a nucleotide incorporated into the rolling circle amplification product during the probe amplification process, or by being linked to a fluorescently labelled oligonucleotide hybridising to the rolling circle amplification product, or wherein said label is a molecule or a chemical group which can be detected by a fluorescently labelled molecule, such as an antibody.

Like nucleotides of 5′ overhang sequences, nucleotides of 3′ overhang sequences can also comprise a nucleobase and a backbone unit, wherein the backbone unit comprises a sugar moiety and an internucleoside linker. The nucleobase of the nucleotides of the 3′ overhang can selected from naturally occurring nucleobases and non-naturally occurring nucleobases. The backbone unit of neighbouring nucleobases can be selected from naturally occurring backbone units and non-naturally occurring backbone units. The sugar moiety of the backbone unit of neighbouring nucleobases can be selected from naturally occurring sugar moieties and non-naturally occurring sugar moieties. The internucleoside linker of the backbone unit of neighbouring nucleobases can be selected from naturally occurring internucleoside linkers and non-naturally occurring internucleoside linkers.

In one embodiment, the nucleobases of the 3′ overhang are selected independently from the group consisting of natural and non-natural purine heterocycles, natural and non-natural pyrimidine heterocycles, including heterocyclic, non-natural analogues and tautomers of said natural purine heterocycles and said natural pyrimidine heterocycles. Accordingly, the nucleobases of the 3′ overhang can e.g. be selected independently from the group consisting of adenine, guanine, isoguanine, thymine, cytosine, isocytosine, pseudoisocytosine, uracil, inosine, purine, xanthine, diaminopurine, 8-oxo-N⁶-methyladenine, 7-deazaxanthine, 7-deazaguanine, N⁴,N⁴-ethanocytosin, N⁶,N⁶-ethano-2,6-diamino-purine, 5-methylcytosine, 5-(C³-C⁶)-alkynylcytosine, 5-fluorouracil, 5-bromouracil and 2-hydroxy-5-methyl-4-triazolopyridine.

The backbone units of the nucleotides of the 3′ overhang can be the same or different backbone units, such as the same or different backbone units selected independently from the group consisting of

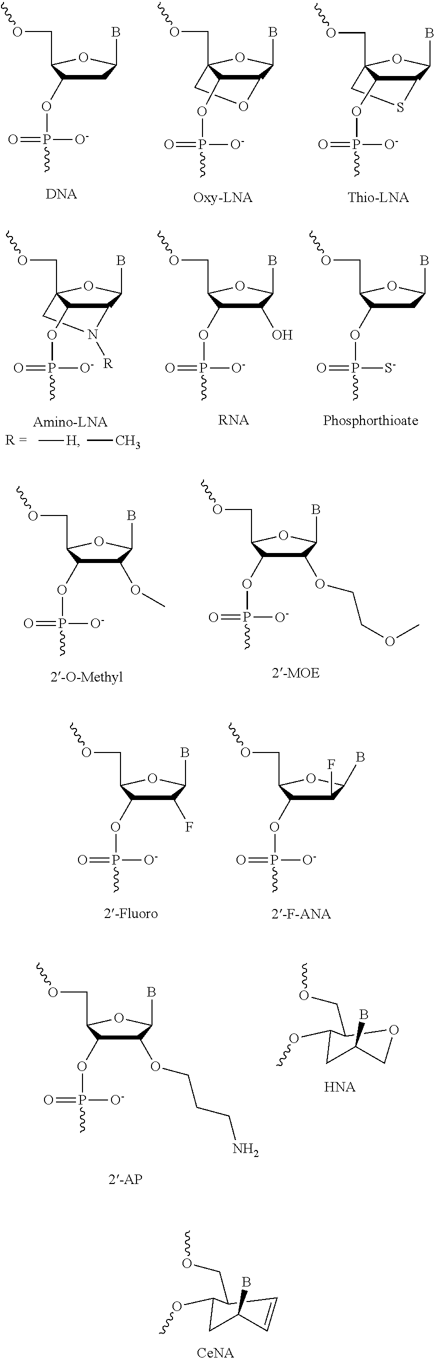

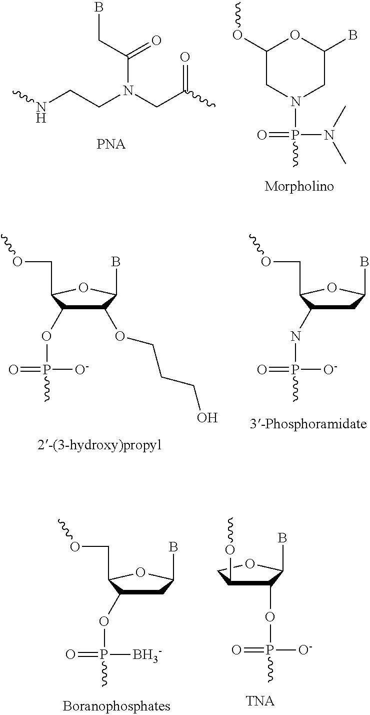

wherein B denotes a nucleobase.

The sugar moiety of the backbone unit of the nucleotides of the 3′ overhang preferably comprises or consists of a pentose, such as a pentose selected from the group consisting of ribose, 2′-deoxyribose, 2′-O-methyl-ribose, 2′-fluor-ribose, and 2′-4′-O-methylene-ribose (LNA). The nucleobase of the nucleotide is preferably attached to the 1′ position of the pentose.

The backbone units linking any two neighbouring nucleotides of the 3′ overhang can be the same or different backbone units, and at least some of the nucleotides of the 3′ overhang can be linked by different backbone units. At least some of said different backbone units can be non-natural backbone units. Also, the internucleoside linkers linking any two neighbouring nucleotides of the 3′ overhang can be the same or different internucleoside linkers. At least some of the nucleotides of the 3′ overhang can be linked by different internucleoside linkers. At least some of said different internucleotide linkers can be non-natural internucleotide linkers.

Generally, the internucleoside linkers of the 3′ overhang can be selected from the group consisting of phosphodiester bonds, phosphorothioate bonds, methylphosphonate bonds, phosphoramidate bonds, phosphotriester bonds and phosphodithioate bonds.

In one embodiment disclosed in more detail herein below, the nucleotides of the 3′ overhang are selected from naturally occurring nucleosides of the DNA and RNA family connected through phosphodiester linkages and at least one non-natural nucleotide selected from the group consisting of nucleotides comprising a non-natural nucleobase and/or a non-natural backbone unit comprising a non-natural sugar moiety and/or a non-natural internucleoside linker. Naturally occurring nucleosides are deoxynucleosides selected from the group consisting of deoxyadenosine, deoxyguanosine, deoxythymidine, and deoxycytidine. Examples of naturally occurring nucleosides are nucleotides selected from the group consisting of adenosine, guanosine, uridine, cytidine, and inosine.

Examples of non-natural nucleobases are nucleobases selected from the group consisting of 8-oxo-N⁶-methyladenine, 7-deazaxanthine, 7-deazaguanine, N⁴,N⁴-ethanocytosin, N⁶,N⁶-ethano-2,6-diamino-purine, 5-methylcytosine, 5-(C³—C⁶)-alkynylcytosine, 5-fluorouracil, 5-bromouracil, pseudoisocytosine, 2-hydroxy-5-methyl-4-triazolopyridine, isocytosine, isoguanine and inosine.

Furthermore, non-natural backbone units of the one or more non-natural nucleotides can be selected from the group consisting of

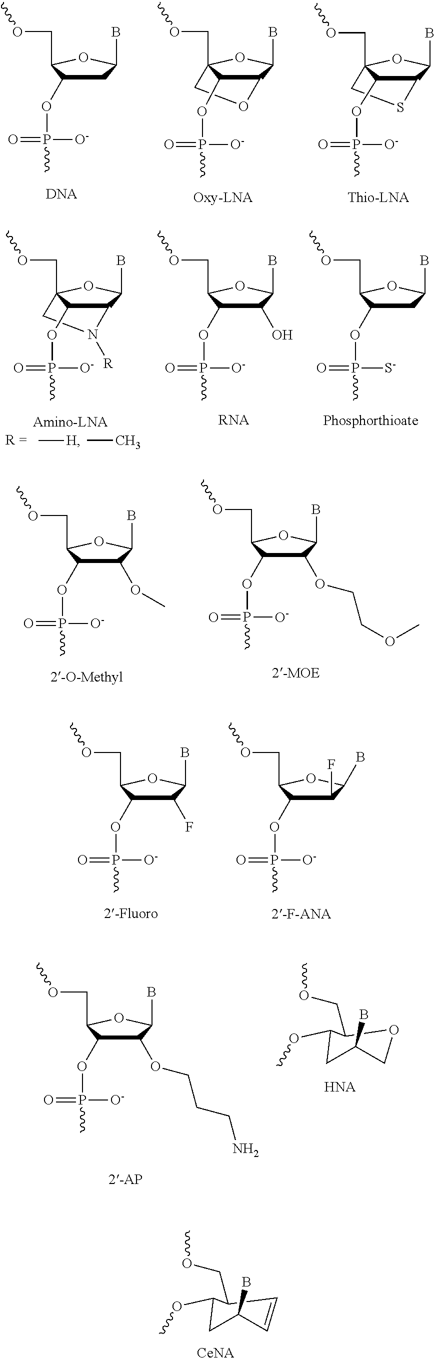

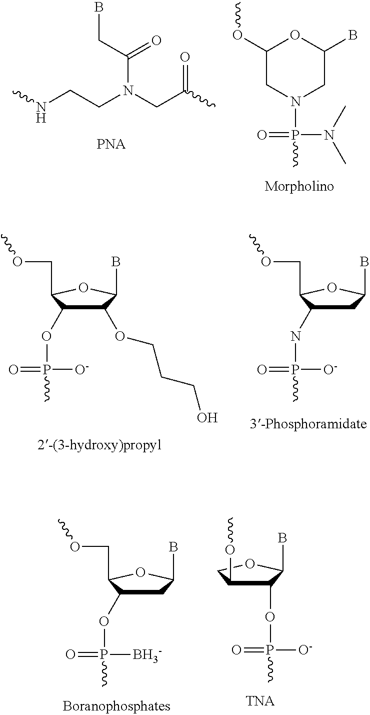

wherein B denotes a nucleobase.

Non-natural sugar moieties of the one or more non-natural backbone unit(s) can be selected from the group consisting of 2′-deoxyribose, 2′-O-methyl-ribose, 2′-fluor-ribose and 2′-4′-O-methylene-ribose (LNA).

Non-natural internucleoside linkers of the one or more non-natural backbone unit(s) can be selected from the group consisting of phosphorothioate bonds, methylphosphonate bonds, phosphoramidate bonds, phosphotriester bonds and phosphodithioate bonds.

In one embodiment disclosed in more detail herein below, the 3′ overhang comprises naturally occurring nucleobases connected by naturally occurring backbone units, wherein said naturally occurring nucleobases and said naturally occurring backbone units do not prevent exonuclease degradation of said 3′ overhang. The 3′ overhang can further comprise non-naturally occurring nucleobases which do not prevent exonuclease degradation of said 3′ overhang, or non-naturally occurring backbone units comprising sugar moieties and internucleoside linkers which do not prevent exonuclease degradation of said 3′ overhang, or non-naturally occurring sugar moieties which do not prevent exonuclease degradation of said 3′ overhang, or non-naturally occurring internucleoside linkers which do not prevent exonuclease degradation of said 3′ overhang.

Accordingly, when said one or more enzyme activities present in said sample comprises a 3′ to 5′ exonuclease activity capable of cleaving one or more, such as all of the internucleoside linkers connecting the nucleotides of the 3′ overhang and/or a ligase activity, a circular oligonucleotide template capable of being amplified by rolling circle amplification is generated by ligating the oligonucleotide probe comprising a substrate moiety processed by 3′ to 5′ exonucleolytically digestion of the 3′ overhang, wherein said ligation is performed by at least one ligase activity present in said sample or added to said sample.

The generated, circular, oligonucleotide template can be amplified by rolling circle amplification, wherein said amplification is indicative of or evidence of the presence in said sample of at least one 3′ to 5′ exonuclease activity and at least one ligase activity. The rolling circle amplification product can detected by detecting a label covalently or non-covalently associated with said rolling circle amplification product, wherein said label is preferably fluorescently detectable, wherein said label is either a fluorescent molecule incorporated into the rolling circle amplification product, for example by being present in the primer used for probe amplification and generation of the rolling circle amplification product, or by being linked to a nucleotide incorporated into the rolling circle amplification product during the probe amplification process, or by being linked to a fluorescently labelled oligonucleotide hybridising to the rolling circle amplification product, or wherein said label is a molecule or a chemical group which can be detected by a fluorescently labelled molecule, such as an antibody.

In another embodiment disclosed in more detail herein below, the 3′ overhang comprises non-naturally occurring nucleobases connected by naturally occurring backbone units and/or non-naturally occurring backbone units, said backbone units comprising a sugar moiety and an internucleoside linker, wherein said non-naturally occurring nucleobases and said non-naturally occurring backbone units, when present, prevent exonuclease degradation of said 3′ overhang. The non-naturally occurring nucleobases alone can prevent exonuclease degradation of said 3′ overhang. The non-naturally occurring backbone units alone can prevent exonuclease degradation of said 3′ overhang. The non-naturally occurring sugar moieties alone can prevent exonuclease degradation of said 3′ overhang. The non-naturally occurring internucleoside linkers alone can prevent exonuclease degradation of said 3′ overhang.

Accordingly, when a 3′ to 5′ exonuclease activity is present in said sample, the activity cannot cleave the internucleoside linkers connecting the nucleotides of the 3′ overhang. Accordingly, when said one or more enzyme activities present in said sample further comprises a topoisomerase II activity capable of processing said unprocessed substrate moiety of said oligonucleotide probe, a circular, oligonucleotide template capable of being amplified by rolling circle amplification is generated by said toposiomerase II activity.

The circular, oligonucleotide template can be amplified by rolling circle amplification, said amplification, which amplification is indicative of the presence in said sample of at least one topoisomerase II activity, wherein said rolling circle amplification product can be detected by detecting a label covalently or non-covalently associated with said rolling circle amplification product, wherein said label is preferably fluorescently detectable, wherein said label is either a fluorescent molecule incorporated into the rolling circle amplification product, for example by being present in the primer used for probe amplification and generation of the rolling circle amplification product, or by being linked to a nucleotide incorporated into the rolling circle amplification product during the probe amplification process, or by being linked to a fluorescently labelled oligonucleotide hybridising to the rolling circle amplification product, or wherein said label is a molecule or a chemical group which can be detected by a fluorescently labelled molecule, such as an antibody.

A number of further embodiments of the present invention is disclosed herein below.

Probe Incubation with Sample

A sample can be provided in several formats such as, but not limited to, cells grown on a surface, cells in solution, cell extracts, tissue preparations or purified enzymes. If the sample is cells on a surface or tissue sections, the probe mixture can be provided by placing the probe mixture in a liquid phase on the cells or tissue sections. Following probe incubation the mixture can be transferred to a solid support before amplification or the amplification can be performed directly on the cell or tissue. If a penetration step is necessary to get the enzymes out of the cells or probes into the cells a pentetration step may be an advantage. This can e.g. be done by hypotonic treatment, detergents, electrophoration, or proteases.

Detergents, such as NP40, triton x-100, tween 20 may be used in the present invention. When using NP40, triton x-100 or tween 20, the concentration is preferably in the range of from about 0.01 to about 2%, from about 0.01 to about 0.05%, from about 0.05 to about 0.1%, from about 0.1 to about 0.2%, from about 0.2 to about 0.3%, from about 0.3 to about 0.4%, from about 0.4 to about 0.5%, from about 0.5 to about 0.6%, from about 0.6 to about 0.7%, from about 0.7 to about 0.8%, from about 0.8 to about 0.9%, from about 0.9 to about 1%, from about 1 to about 1.1%, from about 1.1 to about 1.2%, from about 1.2 to about 1.3%, from about 1.3 to about 1.4%, from about 1.4 to about 1.5%, from about 1.5 to about 1.6%, from about 1.6 to about 1.7%, from about 1.7 to about 1.8%, from about 1.8 to about 1.9%, from about 1.9 to about 2%. SDS can also be used as detergent, albeit at a lower concentration, such as from about 0.001 to about 1%

Cells can also be opened by repeated cycles of freezing and thawing. In a preferred embodiment two cycles of −80° C. to room temperature are performed

Optionally one or more enzymes or chemicals can be added to the sample to stimulate or inhibit the repair event. This could be enzymes such as kinases, ligases, polymerases glycosylases, nucleases, accessory proteins and cofactors or chemicals such as stimulators, inhibitors, NAD+, ATP and dNTPs.

Alternatively, If the sample is a cell culture, the probe can be transfected into the cells by using standard transfection agents such as, but not limited to, lipofectamine, such as FuGene, such as ExGen 500, such as calcium phoshapte, such as electroporation, such as heat shock, such as the gene gun, such as viruses, such as dendrimers, or such as liposomes. Though linear probes are degraded fast inside living cells, circular oligonucleotides are degraded much slower, since most intracellular DNA degradation is caused by exonucleases. Thus, once the probe has been circularized by one or more enzymes it is stabilized. By using this method the enzyme activity, able to circularize the transfected probe, is monitored inside a living cell. Following cell fixation, the circular probes can be detected using rolling circle replication. Thus, in one embodiment of the invention the probes are delivered to the sample by transfections.

If the sample is provided as a cell extract, the sample incubation step can be performed in a test tube, and the probes can subsequently be transferred to a solid support. Alternatively, the probe is pre-bound to a solid support, and the sample in solution is provided to the support.

The sample can be incubated with a concentration of 0.001 μM to 5 μM, preferably 0.01, 1 μM of the probe, in a mixture enabling protein activity. The mixture should have one or more of the following features: be in a buffer such as but not limited to 0.001-0.5M tris-HCl, 0.001×-5×SSC, 0.001×-2×PBS with a pH of such as 2-13, such as 3-12, such as 4-11, such as 4-10, or such as 5-8, preferably the mixture is in 0.1 M tris-HCl pH 7.5, the mixture should contain ATP in a concentration of 0.01 mM to 2 mM, preferably 0.1-1 mM ATP, dNTPs in a concentration of 0.01 mM to 2 mM, preferably 0.1-1 mM dNTPs, one or more of the following ions Mg, K, Ca, Na, Fe, Ni, Cu, and Zn at a concentration of 0.05-500 mM and, one or more reducing agent such as DTT or beta-mercaptoethanol at a concentration of 0.05-100 mM, and one or more protease inhibitors such as but not limited to, AEBSF, aprotinin, bestatin, E-64, leupeptin, pepstatin A and PMSF, preferably a mixture of two more of the inhibitors are used. If other energy sources, such as NAD or NADP, are required the mixture can be supplemented with those. Depending on the reaction setup, one or more of the elements can be left out from the mixture. The probe should be incubated with the sample for an appropriate amount of time, such as one minute to 24 hours, such as five minutes to 16 hours, such as five minutes to four hours, such as five minutes to two hours, such as five minutes to one hour, such as 15 minutes to 30 minutes, or such as 30 minutes. Alternatively, the probe can be hybridized to a primer before incubation with a sample. The primer can either be in solution or bound to a solid support as described below in the section regarding primer design.

### Protease Conditions

Following sample incubation the reaction mix can be inactivated by heating for an appropriate time such as 30 min at 95° C., such as 15 min at 95° C., such as 5 min at 95° C., such as 30 min at 65° C., such as 15 min at 65° C., or such as 5 min at 65° C. Alternatively the proteins in the sample can be inactivated by protease digestion using e.g., but not limited to, proteinase K, pronase or pepsin for such as 60 min at 50° C., such as 30 min at 50° C., such as 15 min at 50° C., such as 60 min at 37° C., such as 30 min at 37° C., or such as 15 min at 37° C.

### Primer Design

In general, a primer consists of 5-50 nucleotides and preferably of 7-15 nucleotides. The primer has to be complementary to part of the nucleic acid probe, preferably a part outside the double stranded region. Preferably the primer is 100% complementary to the probe, alternatively nucleotides at the 5′-end of the primer are non-complementary to the probe, e.g. 1 nucleotide, 3 nucleotides, 5 nucleotides, 10 nucleotides, 25 nucleotides or 50 nucleotides. If a polymerase containing 3′ to 5′ exonuclease activity is used (e.g. Phi29 DNA polymerase), non-complementary nucleotides at the 3′-end of the primer can be present, such as e.g. 1 nucleotide, such as e.g. 3 nucleotides, such as e.g. 5 nucleotides, such as e.g. 10 nucleotides, such as e.g. 25 nucleotides, or such as e.g. 50 nucleotides. Furthermore, mismatched nucleotides in the primer can be present, e.g. 1 nucleotide, such as e.g. 3 nucleotides, such as e.g. 5 nucleotides, such as e.g. 10 nucleotides, or such as e.g. 25 nucleotides. In the case where the probe consists of more than one unbroken chain of nucleotides, one or more of the chains of nucleotides can be used as the primer.

The primer can be synthesized by standard chemical methods (e.g. beta-cyanoethyl phosphoramidite chemistry). A primer can also contain modifications e.g., but not limited to, streptavidine, avidin, biotin, ³²P, and fluorophores, amins or it may comprise artificial nucleotides such as, but not limited to, LNA, PNA, iso-dCTP, and iso-dGTP. For correct annealing between circle and primer, a molar ratio of 0.1-100 between circle and primer is mixed, preferably 0.8-1.2.

It is to be understood that polymerases which do not need a primer can also be used by the method of the invention. In this case no primers are needed to start the rolling circle replication.

The primers, in the method of the invention, can be anchored to a solid support, thereby attaching the following rolling circle product to a surface. This will make it easier to change buffer conditions, and improve washing between the different steps, thereby minimizing background. In one example, the primer can be coupled in the 5′-end to a solid support—a 5′-biotin labeled primer may e.g. be coupled to a streptavidine coated solid support including, but not limited to, PCR-tubes, ELISA plates, beads, plastic CDs (e.g. produced by the company Åmic), and microscope slides. In another example the primer is coupled to a solid support through a 5′-amin, thereby getting a covalent linkage, including, but not limited to, PCR-tubes, ELISA plates, beads, plastic CDs (produced by the company Åmic), and microscope slides. It is to be understood that the primer can also be coupled to a surface when it is part of the probe. Thus, in one aspect, the invention relates to a method, wherein the primer is immobilized on a solid support.

### Probe Hybridization to Primer

The primer can already be present in the sample incubation step, but preferably the primer is added subsequently to sample incubation. The primer can be added together with the polymerase or in an individual hybridization step prior to rolling circle DNA synthesis. If the primer is linked to a solid support, the mixture can be supplemented with 0.01-2 M NaCl (final concentration) to increase hybridization; preferably the mixture is supplemented with 500 mM NaCl (final concentration). If the primer is linked to a solid support, protease digestion can also be performed following probe hybridization to the primer, thereby removing protein cell debris from the solid support.

### Washing Conditions

If the primer is coupled to a solid support and the probe is hybridized to the primer, it is preferable to wash the support before initiation of rolling circle DNA synthesis. In this way the buffer can be changed and unspecific bound cell sample debris can be removed. Several different buffers can be used. Preferably a buffer is removing most unbound sample debris without removing too much of the hybridized probe. Example of washing buffer could be, but not limited to: I) 0.1 M tris-HCl, 150 mM NaCl and 0.5% tween 20. II) 2×SSC and 0.5% tween 20 or III) 0.1 M tris-HCl, 150 mM NaCl and 0.3% SDS. Following washing the slide can either be air-dried or dehydrated through a series of ethanol (e.g. 70%, 85% and 99%) and air-dried.

Alternatively the repair event can be detected by PCR by positioning a primer in each site of the gap.

### Rolling Circle DNA Synthesis

When a polymerase and deoxynucleoside triphosphates (dNTPs) are combined with a probe hybridized to a primer (primer 1) under correct buffer conditions, rolling circle replication can take place. The polymerase will start the polymerization from the 3′-end of the primer, using the circular probe as a rolling-circle-template. As the circular probe is endless, the rolling circle product will comprise a multimer complementary to the sequence of the circular probe. Preferably the polymerase is the Phi29 DNA polymerase. A final concentration of 0.001-2 units of phi29 polymerase (Fermentas) is used, preferably 0.05-1 unit is used. A final dNTP concentration of 0.005-10 mM, preferably 0.1-1 mM is used. Alternatively, other polymerases such as, but not limited to, the T7 DNA polymerase, Sequenase Version 2.0 T7 DNA Polymerase, and Bst DNA polymerase can be used. The incubation time should be between 10 minutes and 24 hours, preferably 30 minutes to 5 hours, at the temperature optimal for the polymerase of choice. For some of the polymerases addition of single stranded binding protein (SSB) enhances the rolling circle activity. Since the Phi29 DNA polymerase is not enhanced by SSB, a concentration of 0 μg/μl SSB is preferably used. Alternatively a concentration of 0.001-0.2 μg/μl can be used. The length of the rolling circle product is preferably between 500 and 500.000 nucleotides in length. The speed and duration of the elongation can be controlled by varying the concentrations of dNTP, polymerase, circle, primer, and SSB. Furthermore, temperature and buffer conditions are adjustable. Following rolling circle from a solid support, it can be washed as described above.

### Detection of Rolling Circle Products

Different methods can be used to identify a specific probe, and the identifier element will differ depending on the choice of method. If detection is obtained through hybridization of a labeled oligonucleotide to the identifier elements, the identifiers need to have a certain length to be specific for a target sequence and allow hybridization under the reaction conditions. In theory an identifier could match the total length of the probe, but in most cases a shorter identifier element would be preferable. Shorter identifiers would have faster hybridization kinetics and would enable a probe to contain more than one identifier.

Thus, in one embodiment, the invention relates to an element defining the specific probe, which is a nucleotide sequence of 6-200 nucleotides, such as e.g. 6-150 nucleotides, or such as e.g. 6-100 nucleotides, or such as e.g. 6-80 nucleotides, or such as e.g. 6-60 nucleotides, or such as e.g. 6-50 nucleotides, or such as e.g. 10-40 nucleotides, or such as e.g. 10-30 nucleotides, or such as e.g. 15-30 nucleotides. However, since the probes are used as templates in rolling circle replications, detection can also be obtained through synthesis. Such detection through synthesis could be performed similar to established linear PRINS reactions. Whereas incorporation of a labeled (e.g. a fluorophore) A, T, G, C, or U is an obvious approach, it will give rise to background staining, as these nucleotides could be incorporated not only in the rolling circle product but also elsewhere in the sample. Incorporating one or more artificial nucleotides, such as isoC or isoG, into the sequence of the probe and providing the complementary nucleotide as a labeled nucleotide (e.g. a fluorophore) during rolling circle DNA synthesis may therefore be preferable. Since such artificial nucleotides are not found in nature, they will not be incorporated to any great extent elsewhere in the sample, minimizing background reactions. This aspect makes the use of a fluorophore-coupled isodCTP nucleotides or iso-dGTP nucleotides preferable. If detection is obtained through synthesis, the identifier element, defining the specific probe, may therefore preferably be one or more artificial nucleotide. Thus, in another embodiment, the invention relates to an element defining the specific probe, which is composed of one or more artificial nucleotides, such as e.g. 1-20 artificial nucleotides, or such as e.g. 1-10 artificial nucleotides, or such as e.g. 1-5 artificial nucleotides, or such as e.g. 4 artificial 30 nucleotides, or such as e.g. 3 artificial nucleotides, or such as e.g. 2 artificial nucleotides, or such as e.g. 1 artificial nucleotide. Thus, each probe can be identified, if desired, by e.g. primer sequence and detection sequence or both.

### Liquid Composition

There is also provided a liquid composition comprising
a) one or more oligonucleotide probes selected from the group consisting of
   i) oligonucleotide probes comprising unprocessed substrate moieties comprising or consisting of one or more nick(s) in one or more single strand(s) of a double stranded nucleotide sequence of said oligonucleotide probe, said one or more nick(s) forming one or more unprocessed substrate moieties of said oligonucleotide probe,
   ii) oligonucleotide probes comprising unprocessed substrate moieties comprising or consisting of one or more single stranded nucleotide sequence(s) joined at one or both ends thereof by a double stranded nucleotide sequence, said single stranded sequence(s) creating one or more gap structure(s) forming one or more unprocessed substrate moieties of said oligonucleotide probe, and
   iii) oligonucleotide probes comprising unprocessed substrate moieties comprising or consisting of one or more nick(s) or one or more gap(s), said gap(s) being in the form of a single stranded nucleotide sequence, said nick(s) or gap(s) being joined at one end thereof to a double stranded nucleotide sequence and at the other end thereof to at least one single stranded overhang joined to a double stranded nucleotide sequence of said oligonucleotide probe, wherein said nick(s) or gap(s) in combination with the at least one single stranded overhang forms one or more unprocessed substrate moieties of said oligonucleotide probe;
   and
b) a liquid carrier, such as an aqueous solvent, allowing one or more enzymes to process the one or more unprocessed substrate moieties of said one or more oligonucleotide probes.

In a further aspect of the present invention there is provided a composition comprising a tissue sample, or a biopsy sample, obtained from an animal, such as a human being, and the above-cited liquid composition.

### Solid Supports According to the Present Invention

In a further aspect of the present invention there is provided a solid support comprising a plurality of attachment points for the attachment to the solid support of one or more oligonucleotide probes each comprising one or more unprocessed substrate moieties, wherein an oligonucleotide probe is either directly attached to an attachment point through one strand of the oligonucleotide probe, wherein said strand is capable of initiating rolling circle amplification of a second strand of the oligonucletide probe, or an oligonucleotide probe is attached to an attachment point through hybridisation of the oligonucleotide probe to a primer oligonucleotide attached to an attachment point, wherein said primer is capable of initiating rolling circle amplification of the oligonucletide probe, so that individual attachment points are associated with one or more oligonucleotide primers suitable for initiating rolling circle amplification of a circular template generated by enzyme processing of said one or more oligonucleotide probes each comprising one or more unprocessed substrate moieties,
wherein the same or different primers are associated with the same or different attachment points,
wherein the oligonucleotide probes attached to the solid support are selected from the group consisting of
   i) oligonucleotide probes comprising unprocessed substrate moieties comprising or consisting of one or more nick(s) in one or more single strand(s) of a double stranded nucleotide sequence of said oligonucleotide probe, said one or more nick(s) forming one or more unprocessed substrate moieties of said oligonucleotide probe,
   ii) oligonucleotide probes comprising unprocessed substrate moieties comprising or consisting of one or more single stranded nucleotide sequence(s) joined at one or both ends thereof by a double stranded nucleotide sequence, said single stranded sequence(s) creating one or more gap structure(s) forming one or more unprocessed substrate moieties of said oligonucleotide probe, and
   iii) oligonucleotide probes comprising unprocessed substrate moieties comprising or consisting of one or more nick(s) or one or more gap(s), said gap(s) being in the form of a single stranded nucleotide sequence, said nick(s) or gap(s) being joined at one end thereof to a double stranded nucleotide sequence and at the other end thereof to at least one single stranded overhang joined to a double stranded nucleotide sequence of said oligonucleotide probe, wherein said nick(s) or gap(s) in combination with the at least one single stranded overhang forms one or more unprocessed substrate moieties of said oligonucleotide probe.

Each oligonucleotide probe attached to an attachment site at a different, predetermined position can comprise the same or a different nucleotide or sequence of nucleotides for use in probe detection and/or probe confirmation. Also, the primer can be associated with one or more label(s) selected from the group consisting of chromophores and fluorophores.

The oligonucleotide probes associated with or attached to the solid support is disclosed herein above.

In one embodiment, there is provided a solid support wherein at least 3 different types of oligonucleotide probes are associated with said solid support through hybridisation to one or more oligonucleotide primers each associated with a solid support attachment point, wherein each of said 3 different types of oligonucleotide probes comprises an unprocessed substrate moiety, wherein the unprocessed substrate moiety of each type of oligonucleotide probe is different and each type of oligonucleotide probe is capable of being processed by at least one different enzyme, wherein said at least one different enzyme is selected from the group consisting of a ligase, an exonuclease, such as a 5′ to 3′ exonuclease or a 3′ to 5′ exonuclease, and an endonuclease, such as a flap endonuclease, such as FEN1 or DNA2P and EXO1, or a topoisomerase, such as a topoisomerase of type I or type II.

The different 3 types of oligonucleotide probes are, in one embodiment,
i) oligonucleotide probes comprising unprocessed substrate moieties comprising or consisting of one or more nick(s) in one or more single strand(s) of a double stranded nucleotide sequence of said oligonucleotide probe, said one or more nick(s) forming one or more unprocessed substrate moieties of said oligonucleotide probe, and
ii) oligonucleotide probes comprising unprocessed substrate moieties comprising or consisting of one or more single stranded nucleotide sequence(s) joined at one or both ends thereof by a double stranded nucleotide sequence, said single stranded sequence(s) creating one or more gap structure(s) forming one or more unprocessed substrate moieties of said oligonucleotide probe, and
iii) oligonucleotide probes comprising unprocessed substrate moieties comprising or consisting of one or more nick(s) or one or more gap(s), said gap(s) being in the form of a single stranded nucleotide sequence, said nick(s) or gap(s) being joined at one end thereof to a double stranded nucleotide sequence and at the other end thereof to at least one single stranded overhang joined to a double stranded nucleotide sequence of said oligonucleotide probe, wherein said nick(s) or gap(s) in combination with the at least one single stranded overhang forms one or more unprocessed substrate moieties of said oligonucleotide probe.

The oligonucleotide probes can be present alone or in any combination. The solid support can further comprise detection means for detection of a rolling circle amplification product generated by amplification of circular oligonucleotide templates generated by substrate moiety processing, wherein said rolling circle amplification generates a rolling circle amplification product which remains associated with an attachment point of said solid support.

The means for detecting the rolling circle amplification product can be any means for detecting a label covalently or non-covalently associated with said rolling circle amplification product, wherein said label is preferably fluorescently detectable, wherein said label is either a fluorescent molecule incorporated into the rolling circle amplification product, for example by being present in the primer used for probe amplification and generation of the rolling circle amplification product, or by being linked to a nucleotide incorporated into the rolling circle amplification product during the probe amplification process, or by being linked to a fluorescently labelled oligonucleotide hybridising to the rolling circle amplification product, or wherein said label is a molecule or a chemical group which can be detected by a fluorescently labelled molecule, such as an antibody.

### Solid Support Comprising RCA Templates

In another aspect of the present invention there is provided a solid support comprising a plurality of attachment points for the attachment of one or more circular oligonucleotide templates to the solid support,
wherein each attachment point is associated with one or more primers suitable for initiating rolling circle amplification of a circular oligonucleotide template generated by enzyme processing of an oligonucleotide probe comprising one or more unprocessed substrate moieties, said processing being performed as disclosed herein elsewhere,
wherein the same or different primers are associated with the same or different attachment points, so that a plurality of circular oligonucleotide templates are attached to the solid support by means of hybridisation of each circular oligonucleotide template to said one or more primers associated with each of said plurality of attachment points,
wherein said circular oligonucleotide templates are selected from the group consisting of
   i) circular oligonucleotide templates resulting from processing and ligation of oligonucleotide probes comprising unprocessed substrate moieties comprising or consisting of one or more nick(s) in one or more single strand(s) of a double stranded nucleotide sequence of said oligonucleotide probe, said one or more nick(s) forming one or more unprocessed substrate moieties of said oligonucleotide probe,
   ii) circular oligonucleotide templates resulting from processing and ligation of oligonucleotide probes comprising unprocessed substrate moieties comprising or consisting of one or more single stranded nucleotide sequence(s) joined at one or both ends thereof by a double stranded nucleotide sequence, said single stranded sequence(s) creating one or more gap structure(s) forming one or more unprocessed substrate moieties of said oligonucleotide probe, and
   iii) circular oligonucleotide templates resulting from processing and ligation of oligonucleotide probes comprising unprocessed substrate moieties comprising or consisting of one or more nick(s) or one or more gap(s), said gap(s) being in the form of a single stranded nucleotide sequence, said nick(s) or gap(s) being joined at one end thereof to a double stranded nucleotide sequence and at the other end thereof to at least one single stranded overhang joined to a double stranded nucleotide sequence of said oligonucleotide probe, wherein said nick(s) or gap(s) in combination with the at least one single stranded overhang forms one or more unprocessed substrate moieties of said oligonucleotide probe.

Each primer attached to an attachment site at a different, predetermined position of the solid support can comprise the same or a different label, and the different labels are preferably selected from the group consisting of chromophores and fluorophores.

### Microfluidic Device

There is also provided a microfluidic device comprising one or more reaction compartments for performing one or more rolling circle amplification events of a circular oligonucleotide template and one or more detection compartments for the detection of said rolling circle amplification events performed in said one or more reaction compartments. The microfluidic device can comprise the solid support according to the present invention.

### Method for Correlation

As the enzyme activities can vary from sample to sample—caused in part by degradation of proteins, changing experimental conditions, time of storage etc., a standard which enables correlation of rolling circle amplification events to the activity of the one or more enzyme activities present in a sample is needed. Accordingly, there is provided, in one embodiment, an external standard, such as a preformed, circular oligonucleotide comprising a different label than the probes being used. This standard would serve as a control for the rolling circle amplification and the detection steps. A build-in control for enzyme activity could be a probe detecting e.g. ligase activity since it is, in theory, the simplest activity to detect and a prerequisite to detecting the nuclease activity.

In this way, the level of ligase activity could serve as an internal standard for the general level of protein activity preserved in the sample. One or more other protein activities could also support this internal control for making a more precise estimate of the general state of protein activity in the sample.

Accordingly, there is provided a method for correlating one or more rolling circle amplification event(s) with the activity of one or more enzymes in a sample, said method comprising the steps of performing the enzyme activity determination methods according to the present invention and amplifying by rolling circle amplification the one or more circular templates having been generated as a result of the presence in said sample of said one or more enzyme activities, wherein the detection of said amplification events is done using the solid support according to the invention or the microfluidic device according to the invention, wherein a predetermined number of rolling circle amplification events correlate with a predetermined enzyme activity, and wherein the actual number of rolling circle amplification events recorded for a given sample is compared to the number of events correlating with said predetermined enzyme activity, thereby correlating the actual number of rolling circle amplification events with said activity of said one or more enzyme activities present in said sample.

### Applications of the Methods and Devices of the Present Invention

In a further aspect there is provided a method for testing the efficacy of a drug or drug-lead, said method comprising the steps of
i) providing a drug or drug-lead to be tested;
ii) providing a biological sample to be treated with the drug or drug-lead;
iii) performing the correlation method of the invention for the biological sample in the absence of drug or drug-lead and determining the activity of one or more enzyme activities involved in circularising a non-circular oligonucleotide probe;
iv) contacting the drug or drug-lead and the biological sample;
v) performing the correlation method of the invention for the biological sample in the presence of drug or drug-lead and determining the activity of one or more enzyme activities involved in circularising a non-circular oligonucleotide probe;
vi) comparing the enzyme activities in the biological sample in the presence and absence, respectively, of the drug or drug-lead, wherein said comparison is obtained by comparing the rolling circle amplification events in the presence and absence, respectively, of the drug or drug-lead, and
vii) evaluating the efficacy of the drug or drug-lead based on the comparison performed in step vi).

The method can be repeated once or more than once.

There is also provided a method for diagnosing or prognosing a disease in an individual by determining the activity of one or more enzyme activities involved in circularising a non-circular oligonucleotide probe, said method comprising the steps of obtaining a biological sample from an individual to be tested, said biological sample comprising said one or more enzyme activities to be tested in the diagnostic or prognostic method,

performing on said biological sample the enzyme activity determination methods according to the invention and amplifying by rolling circle amplification the one or more circular templates having been generated as a result of the presence in said sample of said one or more enzyme activities being tested for, and optionally detecting said amplification events by using the solid support according to the invention or the microfluidic device according to the invention,
determining the number of rolling circle amplification events and correlating said number with a predetermined enzyme activity corresponding to standard defining a physiologically normal activity of the one or more enzyme activities being tested for in a healthy individual,
wherein the actual number of rolling circle amplification events recorded for a given sample is compared to the number of events correlating with said predetermined enzyme activity, thereby correlating the actual number of rolling circle amplification events with said activity of said one or more enzyme activities present in said sample, and diagnosing or prognosing said individual with said disease, or the likelihood of developing said disease, based on the enzyme activities determined in said biological sample.

There is also provided a method for treating a disease diagnosed according to the methods of the present invention, said method comprising the steps of administering a pharmaceutical composition to said individual having being diagnosed with said disease, wherein said medicament is capable of treating said disease by curing the disease or ameliorating the disease.

In another aspect there is provided a method for treating prophylactically a disease prognosed according to the methods of the present invention, said method comprising the steps of administering a pharmaceutical composition to said individual having being prognosed with the likelihood of developing said disease, wherein said pharmaceutical composition is capable of treating prophylactically said disease.

The disease can be any disease comprising an element of genetics involved in nucleotide repair. Examples are cancer and cellular aging.

Accordingly, the cancer disease can be selected from the group consisting of bladder carcinoma, blood (and bone marrow)-hematological malignancies, leukemia, lymphoma, Hodgkin's disease, non-Hodgkin's lymphoma, multiple myeloma, brain tumor, breast cancer, cervical cancer, colorectal cancer—in the colon, rectum, anus, or appendix, esophageal cancer, endometrial cancer—in the uterus, hepatocellular carcinoma—in the liver, gastrointestinal stromal tumor (GIST), laryngeal cancer, lung cancer, mesothelioma—in the pleura or pericardium, oral cancer, osteosarcoma—in bones, ovarian cancer, pancreatic cancer, prostate cancer, renal cell carcinoma—in the kidneys, rhabdomyosarcoma—in muscles, skin cancer (including benign moles and dysplastic nevi), stomach cancer, testicular cancer, and thyroid cancer.

In children or young adults the cancer disease can further be selected from the group consisting of neuroblastoma, leukemia, a cancer in the central nervous system, retinoblastoma, Wilms' tumor, germ cell cancer, soft tissue sarcomas, hepatic cancer, lymphomas, and epithelial cancer.

When related to cellular aging, the disease can be selected from the group consisting of Alzheimer's Disease, Creutzfeld-Jakob Disease, Dementia, Multiple Systems Atrophy, Neurodegenerative Diseases, such as Parkinsonism, Retrogenesis, Sundown Syndrome and Vascular Dementia.

### Solid Supports and Micro-Fluidic Devices

Following the various sample preparation, operations involving the contacting of the sample and the one or more oligonucleotide probes comprising an unprocessed substrate moiety, the nucleotide probes and the sample comprising the enzyme activities to be analysed, or the rolling circle amplification product generated following such contacting, can in one embodiment be subjected to one or more analysis and/or manipulation operations.

Particularly preferred analysis operations include, e.g., rolling circle amplification analyses using a hybridization array comprising an ordered plurality of probe oligonucleotides and/or an analysis based on separation and analysis of rolling circle amplification products further comprising or being associated with a selectively detectable label or a polynucleotide, such as chimeric polynucleotide, further comprising a molecular identifier and/or a selectively detectable label, i.e. analyses using, e.g., microfluidicsic devices such as e.g. microcapillary array electrophoresis.

### Probe Analysis Using a Microfluidics Device Comprising a Hybridization Array

In one embodiment, following sample preparation, the biological sample comprising one or more enzyme activities to be analysed is processed as disclosed herein and the rolling circle amplification products thus obtained are analysed using a hybridization array comprising a plurality of linker oligonucleotides or attachment sites capable of binding to or associating with the nucleotide probes according to the invention.

Accordingly, it shall be understood that the description of analyses of nucleotide templates or rolling circle amplification products using a hybridization array comprising a plurality of ordered oligonucleotides or attachment sites may take place with or without the use of a microfluidics device comprising the array.

Furthermore, when sample processing or rolling circle amplification analysis occurs in one microfluidics device, the processed sample or the rolling circle amplification product may be analysed in said device with or without using a hybridization array comprising an ordered plurality of oligonucleotides or attachment points, or the sample or the nucleotide probe or the rolling circle amplification product may be transferred to another microfluidics device comprising a hybridization array, or the sample or the nucleotide probe or the rolling circle amplification product may be transferred to a hybridization array that does not form part of a microfluidics device.

In one preferred embodiment of the present invention, a microfluidics device, optionally comprising a hybridization array, is used for sample handling or handling of nucleotide template or rolling circle amplification product analysis and characterization.

The method of the present invention for characterizing a nucleotide template or a rolling circle amplification product employs, in one embodiment, a solid support or a microfluidics device wherein the position of the template or of the RCA product is known by means of specific coordinates. Thus, by determining the locations at which nucleotide probes having different, unprocessed substrate moieties or nucleotide templates corresponding thereto, hybridize on the array, or the hybridization pattern, one can determine, following RCA, the templates which have been amplified and thus also the specific enzyme activities present in the sample having undergone analysis.

For example, in preferred embodiments involving diagnostic or prognostic applications, the generation in the hybridization array of RCA products at discrete, predetermined positions will readily confirm the presence of certain enzyme activities in the sample. Likewise, the absence of a RCA product will confirm that a certain enzyme activity is not present in the sample. By reading an output generated by fluorescence label detection or detection of any other form of label, one can readily perform a diagnosis of or prognosis for a given disease condition or disease state.

In one embodiment, the sample comprising at least one enzyme activity to be analysed and one or more nucleotide probes are subjected to mixing, e.g. stirring or shaking. Mixing may be carried out by any method described herein, e.g., through the use of piezoelectric elements, electrophoretic methods, or physical mixing by pumping fluids into and out of a designated reaction chamber, i.e., into an adjoining chamber or channel.

In one embodiment, the detection operation will be performed using a suitable label detection or label reader device external to the diagnostic device. However, it may be desirable in some cases, to incorporate the data gathering operation into the diagnostic device itself. Novel systems for direct detection of RCA events in situ on the array are also encompassed by the present invention.

The data from the RCA detection is analyzed to determine the presence or absence of a particular enzyme activity within the sample. In some cases, probe oligonucleotides or RCA products may be labeled. For example, where biotin labeled dNTPs are used in, e.g., rolling circle amplification, streptavidin linked reporter groups may be used to label the RCA products. Such operations are readily integratable into the systems of the present invention, requiring the use of various mixing methods as is necessary.

### Capillary Electrophoresis

In some embodiments, it may be desirable to provide additional, or alternative means for analyzing the enzymatic activities putatively contained in a sample—by analysing the RCA events resulting from probe nucleotide circularisation and amplification.

Accordingly, in one embodiment, the device of the invention will optionally or additionally comprise a micro capillary array for analysis of the RCA products obtained from amplification of probe nucleotides having reacted with an enzymatic activity of the sample. In this embodiment, the RCA products are capable of being manipulated according to size, molecular weight and/or charge.

Microcapillary array electrophoresis generally involves the use of a thin capillary or channel which may or may not be filled with a particular separation medium. Electrophoresis of a sample through the capillary provides a size based separation profile for the sample. The use of microcapillary electrophoresis in size separation of nucleic acids has been reported in, e.g., Woolley and Mathies, Proc. Nat'l Acad. Sci. USA (1994) 91:11348-11352. Microcapillary array electrophoresis generally provides a rapid method for size based sequencing, PCR product analysis and restriction fragment sizing. The high surface to volume ratio of these capillaries allows for the application of higher electric fields across the capillary without substantial thermal variation across the capillary, consequently allowing for more rapid separations. Furthermore, when combined with confocal imaging methods, these methods provide sensitivity in the range of attomoles, which is comparable to the sensitivity of radioactive sequencing methods.

Microfabrication of microfluidics devices including microcapillary electrophoretic devices has been discussed in detail in, e.g., Jacobsen, et al., Anal. Chem. (1994) 66:1114-1118, Effenhauser, et al., Anal. Chem. (1994) 66:2949-2953, Harrison, et al., Science (1993) 261:895-897, Effenhauser, et al. Anal. Chem. (1993) 65:2637-2642, and Manz, et al., J. Chromatog. (1992) 593:253-258.

Typically, these methods comprise photolithographic etching of micron scale channels on a silica, silicon or other rigid substrate or chip, and can be readily adapted for use in the miniaturized devices of the present invention. In some embodiments, the capillary arrays may be fabricated from the same polymeric materials described for the fabrication of the body of the device, using the injection moulding techniques described herein. In such cases, the capillary and other fluid channels may be moulded into a first planar element. A second thin polymeric member having ports corresponding to the termini of the capillary channels disposed there through, is laminated or sonically welded onto the first to provide the top surface of these channels. Electrodes for electrophoretic control are disposed within these ports/wells for application of the electrical current to the capillary channels. Through use of a relatively thin sheet as the covering member of the capillary channels, heat generated during electrophoresis can be rapidly dissipated. Additionally, the capillary channels may be coated with more thermally conductive material, e.g., glass or ceramic, to enhance heat dissipation.

In many capillary electrophoresis methods, the capillaries, e.g., fused silica capillaries or channels etched, machined or moulded into planar substrates, are filled with an appropriate separation/sieving matrix. Typically, a variety of sieving matrices is known in the art and may be used in the microcapillary arrays. Examples of such matrices include, e.g., hydroxyethyl cellulose, polyacrylamide, agarose and the like. Gel matrices may be introduced and polymerized within the capillary channel. However, in some cases, this may result in entrapment of bubbles within the channels which can interfere with sample separations. Accordingly, it is often desirable to place a preformed separation matrix within the capillary channel(s), prior to mating the planar elements of the capillary portion. Fixing the two parts, e.g., through sonic welding, permanently fixes the matrix within the channel. Polymerization outside of the channels helps to ensure that no bubbles are formed. Further, the pressure of the welding process helps to ensure a void-free system. Generally, the specific gel matrix, running buffers and running conditions are selected to maximize the separation characteristics of the particular application, e.g., the size of the nucleic acid fragments, the required resolution, and the presence of native or undenatured nucleic acid molecules. For example, running buffers may include denaturants, chaotropic agents such as urea or the like, to denature nucleic acids in the sample.

### Data Gathering and RCA Product Analysis

Gathering data from the various analysis operations, e.g., hybridization arrays and/or microcapillary arrays, is carried out using any method known in the art. For example, the arrays may be scanned using lasers to excite fluorescently labeled tags that have been hybridized to or form part of the RCA products, which RCA products can then be imaged using charged coupled devices (“CCDs”) for a wide field scanning of the array. Alternatively, another particularly useful method for gathering data from the arrays is through the use of laser confocal microscopy which combines the ease and speed of a readily automated process with high resolution detection. Particularly preferred scanning devices are generally described in, e.g., U.S. Pat. Nos. 5,143,854 and 5,424,186.

Following the data gathering operation, the data will typically be reported to a data analysis operation. To facilitate the sample analysis operation, the data obtained by the reader from the device will typically be analyzed using a digital computer. Typically, the computer will be appropriately programmed for receipt and storage of the data from the device, as well as for analysis and reporting of the data gathered, i.e., interpreting fluorescence data to determine the identity of nucleotide templates and/or rolling circle amplification products, as well as normalization of background.

### Nucleotide Templates or Rolling Circle Amplification Products Characterization for Diagnostic Purposes

When used for diagnostic purposes, the present invention may in one preferred embodiment exploit a microfluidics device comprising a part used primarily for sample processing purposes and/or analytical purposes, as well as a part used primarily for diagnostic purposes.

A schematic presentation of a representative microfluidics device is disclosed e.g. in U.S. Pat. No. 6,168,948, incorporated herein by reference, wherein the analytical part comprises one or more compartments for sample collection, one or more compartments for sample preparation or sample processing, and one or more compartments for sample analysis, as well as suitable systems for data acquisition, data analysis, and data interpretation. The microfluidics device may further comprise a diagnostic part for performing one or more of the operations of sample collection, preparation and/or analysis using, e.g., rolling circle amplification for the generation of RCA products, identification of said RCA products and/or separation of RCA products according to size, molecular weight, or charge.

The diagnostic part of the device can be connected to a reader device in order to detect the hybridization and/or separation information contained in the device. The hybridization and/or separation data is reported from the reader device to a computer which is programmed with appropriate software for interpreting the data obtained by the reader device from the diagnostic device.

Interpretation of the data from the diagnostic device may be used in a variety of ways, such as for nucleotide template identification and/or rolling circle amplification product identification directed towards a particular disease or genetic disorders, such as e.g., cancer, deficiencies associated with the immune system, diabetes, fibrotic diseases, thrombotic diseases, Alzheimer's disease, sickle cell anemia, cystic fibrosis, Fragile X syndrome and Duchenne muscular dystrophy. For each of the afore-mentioned diseases, an increased or decreased or aberant enzyme activity in a tested sample is indicative of the disease in the individual from whom the sample is obtained.

### Fen1

Several Proteins are Known to Interact with Fen1, and through this Interaction Regulate the Activity of Fen1

TABLE 1
Interaction partners of Fen1^{a}

| Protein | Role of interacting protein | Genome maintenance mechanism affected | Refs |
|---|---|---|---|
| Dna2 | Removal of RNA-containing primers of Okazakl fragments | DNA replication | [25] |
| | Hellcase, endonuclease activities | | |
| HIV-1 integrase | DNA binding, specific endonuclease activity, DNA | DNA repair of HIV-1 integration | [59] |
| | joining, disintegration | intermediates | |
| RP-A | Component of DNA replication, DNA repair | DNA replication | [60] |
| | and recombination apparatus | | |
| | Single-strand binding protein, unwinding activity | | |
| PCNA | Component of DNA replication, DNA repair | DNA replication | [61] |
| | and recombination apparatus | | |
| | | DNA repair (BER) | |
| AP endonuclease 1 | Incision of the AP site | DNA repair (BER) | [62] |
| Werner syndrome protein | Hellcase activity | DNA repelication | [38] |
| | | DNA repair | |
| p300 | Transcriptional coactivator | DNA repair/DNA replication? | [20] |
| | Acetyltransferase acitivity | DNA binding and nuclease activity of | |
| | | Fen1 reduced | |
| Cdk1, Cdk2 | Cyclin-dependent kinases | DNA replication (end of S phase) | [21] |
| Cyclin A | Once-per-cell-cycle control of DNA replication | Reduced Fen1 nuclease activity | [21] |

^{a}Fen1, Sap endonuclease 1: HIV-1, human immenodeSciency virus-1: RP-A. replication protein A: PCNA, proliferating cell nuclear antigen; AP, apuriniclapyrimidinic; SER, base excision repair.

In one embodiment, the present invention provides a microfluidics device and RCA based methods for monitoring treatment of a neoplastic disease, such as cancer. Often the efficacy of a cancer drug is tested in a clinical trial to test whether a new treatment has an anti-cancer effect, for example, whether it shrinks a tumour or improves blood test results, and whether it works against a certain type of cancer. A tumour is an abnormal mass of tissue that results from excessive cell division (mitotic activity). Tumors perform no useful body function and may be either benign or malignant. Malignant tumours are cancerous and grow with a tendency to invade and destroy nearby tissue and spread to other parts of the body through the bloodstream and lymphatic system. This is termed metastasis. Cancer cells also avoid natural cell death (apoptosis). Neoplastic diseases as used herein includes any abnormal and uncontrolled cell growth (mitosis) that results in the production of a tumour (i.e. a neoplasm). Neoplasitc diseases include diseases wherein a malignant tumour grows with a tendency to invade and destroy nearby tissue and spread to other parts of the body through the bloodstream and lymphatic system. Cancers can be classified by the type of cell in which it originates and by the location of the cell. Accordingly, Carcinomas originate in epithelial cells, e.g. skin, digestive tract or glands. Leukemia starts in the bone marrow stem cells. Lymphoma is a cancer originating in lymphatic tissue. Melanoma arises in melanocytes. Sarcoma begins in the connective tissue of bone or muscle. Teratoma begins within germ cells. Adult cancers usually form in epithelial tissues and are believed often to be the result of a long biological process related to the interaction of exogenous exposures with genetic and other endogenous characteristics among susceptible people. Examples include: bladder carcinoma, blood (and bone marrow)-hematological malignancies, leukemia, lymphoma, Hodgkin's disease, non-Hodgkin's lymphoma, multiple myeloma, brain tumor, breast cancer, cervical cancer, colorectal cancer—in the colon, rectum, anus, or appendix, esophageal cancer, endometrial cancer—in the uterus, hepatocellular carcinoma—in the liver, gastrointestinal stromal tumor (GIST), laryngeal cancer, lung cancer, mesothelioma—in the pleura or pericardium, oral cancer, osteosarcoma—in bones, ovarian cancer, pancreatic cancer, prostate cancer, renal cell carcinoma—in the kidneys, rhabdomyosarcoma—in muscles, skin cancer (including benign moles and dysplastic nevi), stomach cancer, testicular cancer, and thyroid cancer. Cancer can also occur in young children, particularly infants. Childhood cancers include, from most frequently occurring to least: Neuroblastoma, leukemia, central nervous system, retinoblastoma, Wilms' tumor, germ cell, soft tissue sarcomas, hepatic, lymphomas, epithelial.

In one embodiment, the present invention provides a microfluidics device and RCA based methods for monitoring diseases of the immune system. Often the efficacy of a drug for stimulating the immune system is tested in a clinical trial to test whether a new treatment is capable of stimulating or restorating the ability of the immune system to fight infection and disease. Drugs putatively capable of reducing or eliminating any side effect(s) that may be caused by some cancer treatments can also be monitored.

When used for diagnostic and/or analytical purposes, including characterization of nucleotide templates and/or rolling circle amplification products, the device generally comprises a number of discrete reaction, storage and/or analytical chambers disposed within a single unit or body. While referred to herein as a “diagnostic device,” those of skill in the art will appreciate that the device of the invention will have a variety of applications outside the scope of diagnostics alone. Such applications include sample identification and characterization applications (for, e.g., diagnostic tests, prognostic tests, taxonomic studies, forensic applications, i.e., criminal investigations, and the like).

Typically, the body of the device defines the various reaction chambers and fluid passages in which the above described operations are carried out. Fabrication of the body, and thus the various chambers and channels disposed within the body may generally be carried out using one or a combination of a variety of well known manufacturing techniques and materials. Generally, the material from which the body is fabricated will be selected so as to provide maximum resistance to the full range of conditions to which the device will be exposed, e.g., extremes of temperature, salt, pH, application of electric fields and the like, and will also be selected for compatibility with other materials used in the device. Additional components may be later introduced, as necessary, into the body. Alternatively, the device may be formed from a plurality of distinct parts that are later assembled or mated. For example, separate and individual chambers and fluid passages may be assembled to provide the various chambers of the device.

As a miniaturized device, the body of the microfluidics device as described herein will typically be approximately 1 to 20 cm in length by about 1 to 10 cm in width by about 0.1 cm to about 2 cm thick. Although indicative of a rectangular shape, it will be readily appreciated that the devices of the invention may be embodied in any number of shapes depending upon the particular need. Additionally, these dimensions will typically vary depending upon the number of operations to be performed by the device, the complexity of these operations and the like. As a result, these dimensions are provided as a general indication of the size of the device.

The number and size of the reaction chambers included within the device will also vary depending upon the specific application for which the device is to be used. Generally, the device will include at least two distinct reaction chambers, and preferably, at least three, four or five distinct reaction chambers, all integrated within a single body. Individual reaction chambers will also vary in size and shape according to the specific function(s) of the reaction chamber.

For example, in some cases, circular reaction chambers may be employed. Alternatively, elongate reaction chambers may be used. In general however, the reaction chambers will be from about 0.05 mm to about 20 mm in width or diameter, preferably from about 0.1 mm to about 2.0 mm in width or diameter and about 0.05 mm to about 5 mm deep, and preferably 0.05 mm to about 1 mm deep. For elongate chambers, length will also typically vary along these same ranges.

Microfluidics channels, on the other hand, are typically distinguished from chambers in having smaller dimensions relative to the chambers, and will typically range from about 10 μm to about 1000 μm wide, preferably, 100 μm to 500 μm wide and about 1 μm to 500 μm deep. Although described in terms of reaction chambers, it will be appreciated that these chambers may perform a number of varied functions, e.g., as storage chambers, incubation chambers, mixing chambers and the like.

In some cases, a separate chamber or chambers may be used as volumetric chambers, e.g., to precisely measure fluid volumes for introduction into a subsequent reaction chamber. In such cases, the volume of the chamber will be dictated by volumetric needs of a given reaction. Further, the device may be fabricated to include a range of volumetric chambers having varied, but known volumes or volume ratios (e.g., in comparison to a reaction chamber or other volumetric chambers).

As described above, the body of the device is generally fabricated using one or more of a variety of methods and materials suitable for microfabrication techniques. For example, in preferred aspects, the body of the device may comprise a number of planar members that may individually be injection molded parts fabricated from a variety of polymeric materials, or may be silicon, glass, or the like. In the case of substrates like silica, glass or silicon, methods for etching, milling, drilling, etc., may be used to produce wells and depressions which make up the various reaction chambers and fluid channels within the device.

Microfabrication techniques, such as those regularly used in the semiconductor and microelectronics industries are particularly suited to these materials and methods. These techniques include, e.g., electrodeposition, low-pressure vapor deposition, photolithography, wet chemical etching, reactive ion etching (RIE), laser drilling, and the like. Where these methods are used, it will generally be desirable to fabricate the planar members of the device from materials similar to those used in the semiconductor industry, i.e., silica, silicon, gallium arsenide, polyimide substrates. U.S. Pat. No. 5,252,294, to Kroy, et al., incorporated herein by reference in its entirety for all purposes, reports the fabrication of a silicon based multiwell apparatus for sample handling in biotechnology applications.

Photolithographic methods of etching substrates are particularly well suited for the microfabrication of these substrates and are well known in the art. For example, the first sheet of a substrate may be overlaid with a photoresist. An electromagnetic radiation source may then be shone through a photolithographic mask to expose the photoresist in a pattern which reflects the pattern of chambers and/or channels on the surface of the sheet. After removing the exposed photoresist, the exposed substrate may be etched to produce the desired wells and channels. Generally preferred photoresists include those used extensively in the semiconductor industry. Such materials include polymethyl methacrylate (PMMA) and its derivatives, and electron beam resists such as poly(olefin sulfones) and the like (more fully discussed in, e.g., Ghandi, “VLSI Fabrication Principles,” Wiley (1983) Chapter 10, incorporated herein by reference in its entirety for all purposes).

As an example, the wells manufactured into the surface of one planar member make up the various reaction chambers of the device. Channels manufactured into the surface of this or another planar member make up fluid channels which are used to fluidly connect the various reaction chambers. Another planar member is then placed over and bonded to the first, whereby the wells in the first planar member define cavities within the body of the device which cavities are the various reaction chambers of the device. Similarly, fluid channels manufactured in the surface of one planar member, when covered with a second planar member define fluid passages through the body of the device. These planar members are bonded together or laminated to produce a fluid tight body of the device.

Bonding of the planar members of the device may generally be carried out using a variety of methods known in the art and which may vary depending upon the materials used. For example, adhesives may generally be used to bond the planar members together. Where the planar members are, e.g., glass, silicon or combinations thereof, thermal bonding, anodic/electrostatic or silicon fusion bonding methods may be applied. For polymeric parts, a similar variety of methods may be employed in coupling substrate parts together, e.g., heat with pressure, solvent based bonding. Generally, acoustic welding techniques are generally preferred. In a related aspect, adhesive tapes may be employed as one portion of the device forming a thin wall of the reaction chamber/channel structures.

Although primarily described in terms of producing a fully integrated body of the device, the above described methods can also be used to fabricate individual discrete components of the device which are later assembled into the body of the device.

In additional embodiments, the body may comprise a combination of materials and manufacturing techniques described above. In some cases, the body may include some parts of injection molded plastics, and the like, while other portions of the body may comprise etched silica or silicon planar members, and the like. For example, injection molding techniques may be used to form a number of discrete cavities in a planar surface which define the various reaction chambers, whereas additional components, e.g., fluid channels, arrays, etc, may be fabricated on a planar glass, silica or silicon chip or substrate. Lamination of one set of parts to the other will then result in the formation of the various reaction chambers, interconnected by the appropriate fluid channels.

In particularly preferred embodiments, the body of the device is made from at least one injection molded, press molded or machined polymeric part that has one or more wells or depressions manufactured into its surface to define several of the walls of the reaction chamber or chambers. Molds or mold faces for producing these injection molded parts may generally be fabricated using the methods described herein for, e.g., conventional machining or silicon molds. Examples of suitable polymers for injection molding or machining include, e.g., polycarbonate, polystyrene, polypropylene, polyethylene, acrylic, and commercial polymers such as Kapton, Valox, Teflon, ABS, Delrin and the like. A second part that is similarly planar in shape is mated to the surface of the polymeric part to define the remaining wall of the reaction chamber(s). Published PCT Application No. 95/33846, incorporated herein by reference, describes a device that is used to package individual hybridization array comprising a plurality of ordered first and/or second identifying linker oligonucleotides. The device includes a hybridization chamber disposed within a planar body. The chamber is fluidly connected to an inlet port and an outlet port via flow channels in the body of the device. The body includes a plurality of injection moulded planar parts that are mated to form the body of the device, and which define the flow channels and hybridization chamber.

The surfaces of the fluid channels and reaction chambers which contact the samples and reagents may also be modified to better accommodate a desired reaction. Surfaces may be made more hydrophobic or more hydrophilic depending upon the particular application. Alternatively, surfaces may be coated with any number of materials in order to make the overall system more compatible to the reactions being carried out. For example, in the case of nucleic acid analyses, it may be desirable to coat the surfaces with a non-stick coating, e.g., a Teflon, parylene or silicon, to prevent adhesion of nucleic acids to the surface. Additionally, insulator coatings may also be desirable in those instances where electrical leads are placed in contact with fluids, to prevent shorting out, or excess gas formation from electrolysis. Such insulators may include those well known in the art, e.g., silicon oxide, ceramics or the like.

### DEFINITIONS

Gap: A double stranded region of DNA wherein one of the strands possesses a free 5′-end and a free 3′-end separated by a gap of one or more nucleotides.

Nick: A double stranded region of DNA wherein one of the strands possesses a free 5′-end and a free 3′-end separated by a gap of zero nucleotides.

Artificial nucleic acid: That being both nucleic acids not found in the nature, e.g. but not limited to, PNA, LNA, iso-dCTP, or iso-dGTP, as well as any modified nucleotide, e.g., but not limited to, biotin coupled nucleotides, fluorophore coupled nucleotides, or radioactive nucleotides.

Closed circular structure: A nucleic acid sequence with a non-ending backbone, e.g., but not limited to, sugar-phosphate in DNA and RNA, or N-(2-aminoethyl)-glycine units linked by peptide bonds in PNA.

Hybridize: Base pairing between two complementary nucleic acid sequences.

Intra-molecular structure: Hybridisation of one or more nucleic acid sequence parts in a molecule to one or more nucleic acid sequence parts of the same molecule.

LNA: Locked nucleic acid.

Natural nucleic acids: The nucleotides G, C, A, T, U and I.

Open circular structure: A nucleic acid sequence which is in a circular structure, either aided by an external ligation template or self-templated, with at least one 5′-end and one 3′-end. E.g., but not limited to, sugar-phosphate in DNA and RNA, or N-(2-aminoethyl)-glycine units linked by peptide bonds in PNA.

PNA: Peptide nucleic acid.

Probe: A nucleic acid sequence composed of natural or artificial, modified or unmodified nucleotides, having a length of e.g. 6-200 nucleotides.

Rolling circle template: A closed circular sequence of nucleotides, artificial or natural, that the polymerase uses as a template during rolling circle replication.

Rolling circle DNA synthesis: DNA synthesis using a circular single stranded oligonucleotide as rolling circle template and a target RNA molecule as primer. The addition of a DNA polymerase and dNTPs starts the polymerization. As the rolling circle template is endless, the product will be a long single stranded DNA strand composed of tandem repeats complementary to the rolling circle template. Artificial as well as natural nucleic acid residues can serve as substrates for the rolling circle replication.

### BRIEF DESCRIPTION OF THE DRAWINGS

FIG. 1—Schematic representation of the protein activity assays using self-templating probes.

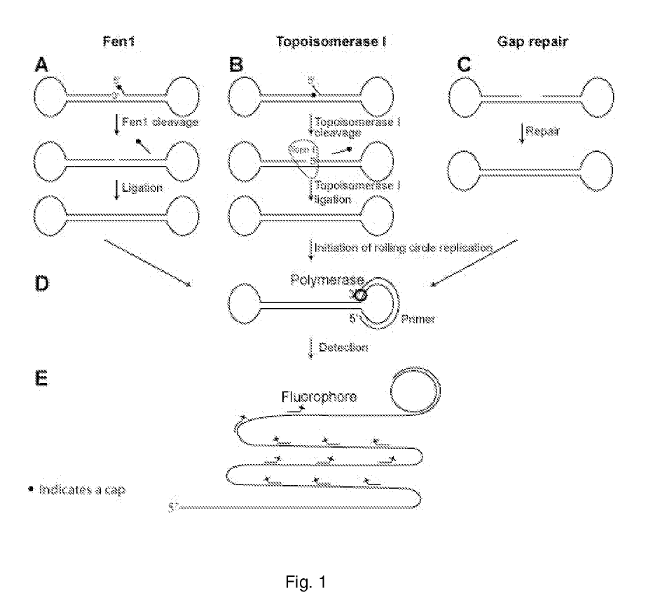

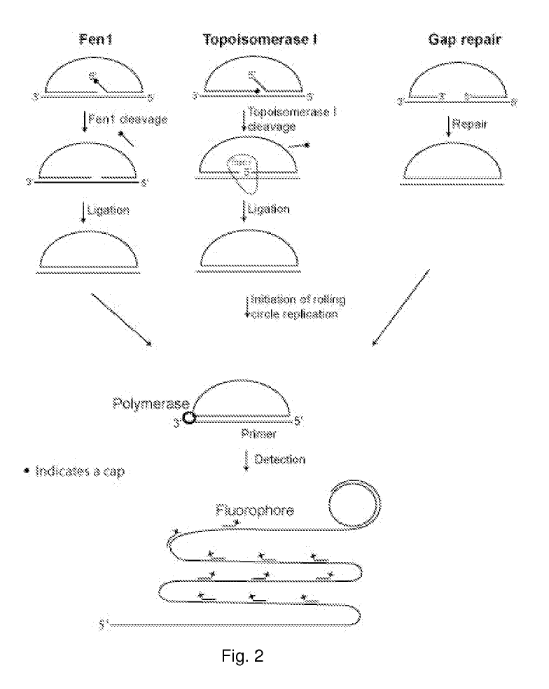

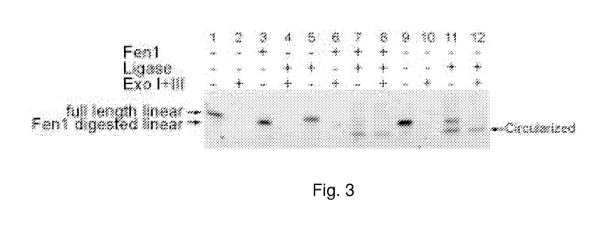

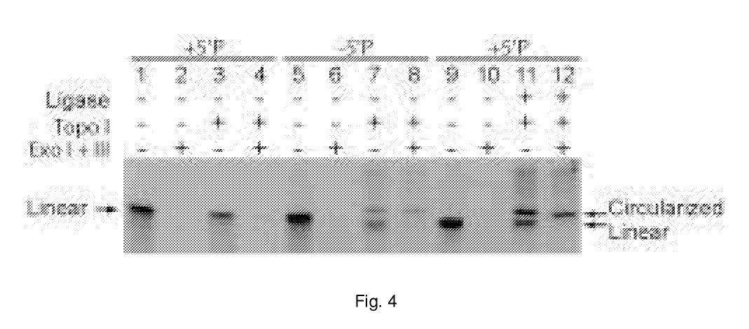

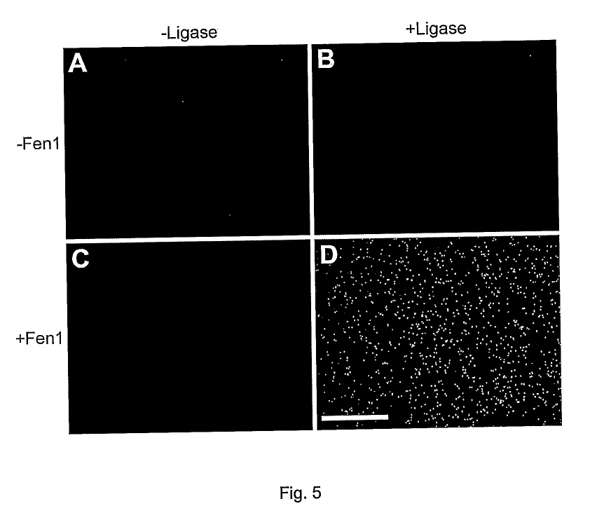

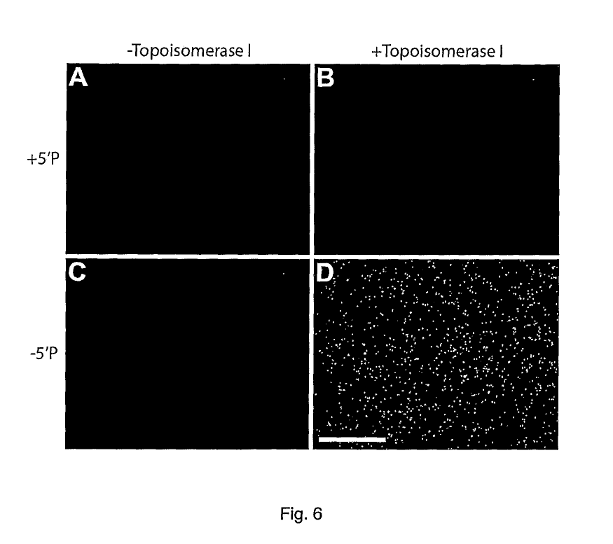

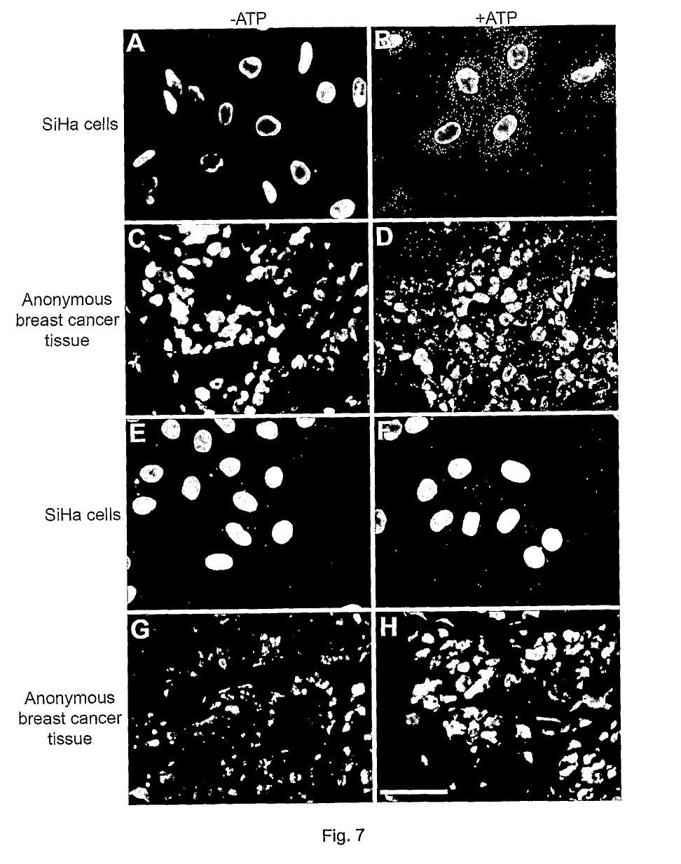

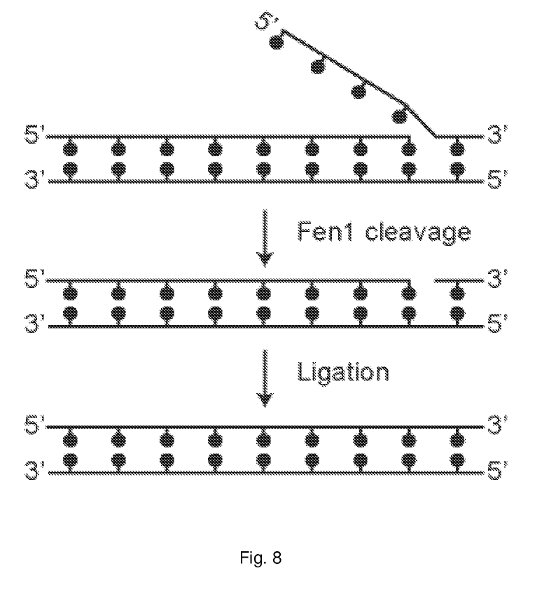

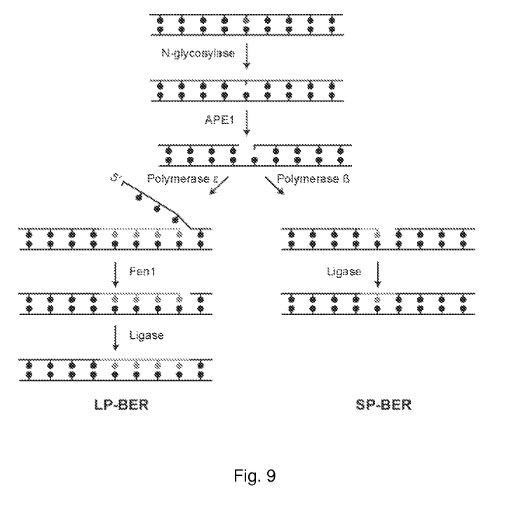

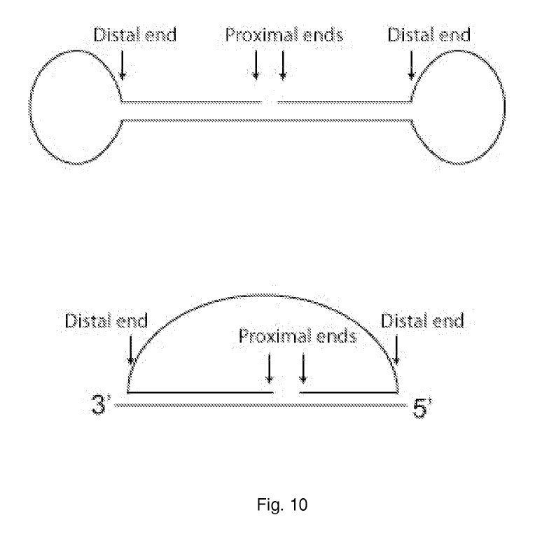

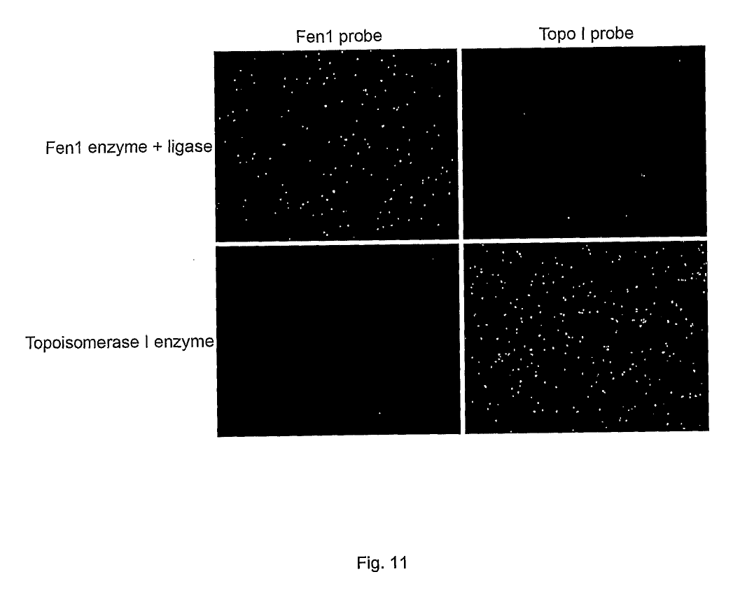

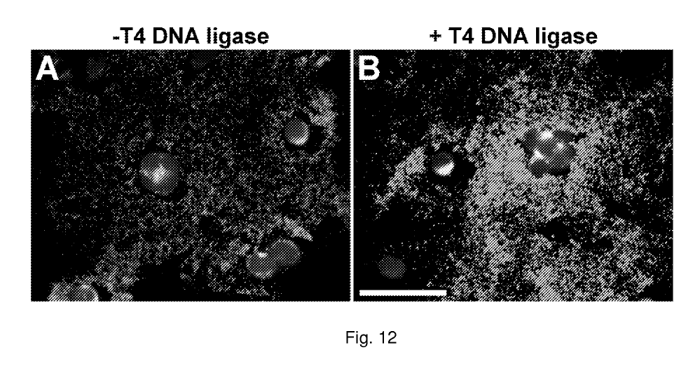

Each probe contains a detection sequence, a primer hybridization sequence and a double stranded region unique for the probe. To increase the specificity of individual probes, modifications can be inserted at both the 5′-end and the 3′-end. (A) In step 1 Fen1 recognizes the flap structure and removes the overhang at the base of the flap, thereby preparing the probe for the ligase, which can circularize the probe. (B) Topoisomerase I cleaves the probe three nucleotides away from the 3′-end, creating a covalent phospho-tyrosine intermediate. The three far most nucleotides are removed and the new 3′-end is ligated to the 5′-end. Following circularization the products can be visualized in a gel or by rolling circle replication. (C) The rolling circle replication is initiated by the presence of primer and a polymerase (It is to be understood that the primer can be either in solution or linked to a solid support. Furthermore, the probe can be hybridized to the primer either before or after incubation with the sample). (D) The rolling circle product is visualized by hybridization of a labeled oligonucleotide to the detection sequence. • indicates a biotin for the Fen1 probe and an amin for the Topo I probe.

FIG. 2—Schematic representation of the protein activity assays using probes comprising two nucleotide sequences.

Each probe contains a detection sequence, a primer hybridization sequence and a double stranded region unique for the probe. To increase the specificity of individual probes, modifications can be inserted at both the 5′-end and the 3′-end. (A) In step 1 Fen1 recognizes the flap structure and removes the overhang at the base of the flap, thereby preparing the probe for the ligase, which can circularize the probe. (B) Topoisomerase I cleaves the probe three nucleotides away from the 3′-end, creating a covalent phospho-tyrosine intermediate. The three far most nucleotides are removed and the new 3′-end is ligated to the 5′-end. Following circularization the products can be visualized in a gel or by rolling circle replication. (C) The rolling circle replication is initiated by the presence of primer and a polymerase (it is to be understood the part of the probe which functions as the primer can be either in solution or linked to a solid support). (D) The rolling circle product is visualized by hybridization of a labeled oligonucleotide to the detection sequence. • indicates a biotin for the Fen1 probe and an amin for the Topo I probe.

FIG. 3-Gel-based detection of Fen1 activity.

Lane 1: Fen1 probe. Lane 2: Same as lane1+exonuclease I and III digestion. Lane 3: Fen1 cleaved Fen1 probe. Lane 4: Same as lane3+exonuclease I and III digestion. Lane 5: Fen1 probe+T4 DNA ligase. Lane 6: Same as lane5+exonuclease I and III digestion. Lane 7: Fen1 digested Fen1 probe+T4 DNA ligase. Lane 8: Same as lane7+exonuclease I and III digestion. Lane 9: Fen1 POS probe. Lane 10: Same as lane9+exonuclease I and III digestion. Lane 11: Fen1 POS probe+T4 DNA ligase. Lane 12: Same as lane11+exonuclease I and III digestion. The gel was stained with SYBR Gold.

FIG. 4—Gel-based detection of topoisomerase I activity.

Lane 1: Topo I probe +5′P. Lane 2: Same as lane1+exonuclease I and III digestion. Lane 3: Topo I probe +5′P+topoisomerase I. Lane 4: Same as lane3+exonuclease I and III digestion. Lane 5: Topo I probe −5′P. Lane 6: Same as lane5+exonuclease I and III digestion. Lane 7: Topo I probe −5′P+topoisomerase I. Lane 8: Same as lane7+exonuclease I and III digestion. Lane 9: Topo I POS probe +5′P. Lane 10: Same as lane9+exonuclease I and III digestion. Lane 11: Topo I POS probe +5′P+T4 DNA ligase. Lane 12: Same as lane 11+exonuclease I and III digestion. The gel was stained with SYBR Gold.

FIG. 5—Fen1 protein activity detection by solid support rolling circle replication.

The Fen1 probe was applied in A-D. (A) −Fen1, −T4 DNA ligase. (B) −Fen1, +T4 DNA ligase. (C) +Fen1, −T4 DNA ligase. (D) +Fen1, +T4 DNA ligase. Scale bar, 100 μm.

FIG. 6—Topoisomerase I protein activity detection by solid support rolling circle replication.

The topoisomerase I probe was applied in A-D. (A) +5′P, −topoisomerase I. (B) +5′P, +topoisomerase I. (C) −5′P, −topoisomerase I. (D) −5′P, +topoisomerase I. Scale bar, 100 μm.

FIG. 7—Enzyme activity detection directly on cells and tissue.

(A-D) Fen1 probe. (A) SiHa cells, −ATP. (B) SiHa cells +ATP. (C) Tissue, −ATP. (D) Tissue +ATP. (E-H) Topo I probe. (E) SiHa cells, −ATP. (F) SiHa cells +ATP. (G) Tissue, −ATP. (H) Tissue +ATP. Scale bar, 100 μm.

FIG. 8—Fen1 endonucleolytic activity.

Fen1 recognizes 5′-flap structures, scans from the 5′-end until it reaches the base of the flap. Following endonucleolytic cleavage the substrate is prepared for ligation. Black circles symbolize bases.

FIG. 9: Simplified illustration of LP-BER and SP-BER. Both pathways start with the removal of a damaged base by a glycosylase and APE1 makes a nick 5′ to the abasic site. In SP-BER polymerase 13 removes the sugar group and inserts the missing nucleotide thereby preparing the substrate for ligation. In LP-BER polymerase ε makes strand displacement thereby inserting several nucleotides. Fen1 removes the displaced strand preparing the substrate for ligation. Black circles symbolize a base. Star indicates a damaged base. Gray circles symbolize a repaired base.

FIG. 10: Explanation of proximal and distal ends of the probes.

FIG. 11: Specificity of topo I probe and Fen1 probe. The Fen1 probe is inert to circularization by topoisomerase I and the topo I probe is inert to circularization by a combination of Fen1 and T4 DNA ligase.

FIG. 12: Stimulation of the circularization of the Fen1 probe on SiHa-cells, by the addition of T4 DNA ligase.

Adding T4 DNA ligase to the reaction mixture results in an increase in the number of obtained signals.

### REFERENCES

Ames B N, Shigenaga M K, Hagen T M. 1993. Oxidants, antioxidants, and the degenerative diseases of aging. Proc Natl Acad Sci USA 90:7915-7922.
Andersen A H, Gocke E, Bonven B J, Nielsen O F, Westergaard O. 1985. Topoisomerase I has a strong binding preference for a conserved hexadecameric sequence in the promoter region of the rRNA gene from Tetrahymena pyriformis. Nucleic Acids Res 13:1543-1557.
Champoux J J. 2001. DNA topoisomerases: structure, function, and mechanism. Annu Rev Biochem 70:369-413.
Collins A R. 2004. The comet assay for DNA damage and repair: principles, applications, and limitations. Mol Biotechnol 26:249-261.
Dizdaroglu M. 2005. Base-excision repair of oxidative DNA damage by DNA glycosylases. Mutat Res 591:45-59.
Fire A, Xu S Q. 1995. Rolling replication of short DNA circles. Proc Natl Acad Sci USA 92:4641-4645.
Fredriksson S, Gullberg M, Jarvius J, Olsson C, Pietras K, Gustafsdottir S M, Ostman A, Landegren U. 2002. Protein detection using proximity-dependent DNA ligation assays. Nat Biotechnol 20:473-477.
Friedrich-Heineken E, Hubscher U. 2004. The Fen1 extrahelical 3′-flap pocket is conserved from archaea to human and regulates DNA substrate specificity. Nucleic Acids Res 32:2520-2528.
Harrington J J, Lieber M R. 1994. The characterization of a mammalian DNA structure-specific endonuclease. Embo J 13:1235-1246.
Hasan S, Stucki M, Hassa P O, Imhof R, Gehrig P, Hunziker P, Hubscher U, Hottiger M O. 2001. Regulation of human flap endonuclease-1 activity by acetylation through the transcriptional coactivator p300. Mol Cell 7:1221-1231.
Henneke G, Koundrioukoff S, Hubscher U. 2003. Phosphorylation of human Fen1 by cyclin-dependent kinase modulates its role in replication fork regulation. Oncogene 22:4301-4313.
Ide H, Kotera M. 2004. Human DNA glycosylases involved in the repair of oxidatively damaged DNA. Biol Pharm Bull 27:480-485.
Iwai S. 2006. Chemical synthesis of oligonucleotides containing damaged bases for biological studies. Nucleosides Nucleotides Nucleic Acids 25:561-582.

Klungland A, Lindahl T. 1997. Second pathway for completion of human DNA base excision-repair: reconstitution with purified proteins and requirement for DNase IV (FEN1). Embo J 16:3341-3348.
Koch J E, Kolvraa S, Petersen K B, Gregersen N, Bolund L. 1989. Oligonucleotide-priming methods for the chromosome-specific labelling of alpha satellite DNA in situ. Chromosoma 98:259-265.
Kucherlapati M, Yang K, Kuraguchi M, Zhao J, Lia M, Heyer J, Kane M F, Fan K, Russell R, Brown A M, Kneitz B, Edelmann W, Kolodner R D, Lipkin M, Kucherlapati R. 2002. Haploinsufficiency of Flap endonuclease (Fen1) leads to rapid tumor progression. Proc Natl Acad Sci USA 99:9924-9929.
Larsen E, Gran C, Saether B E, Seeberg E, Klungland A. 2003. Proliferation failure and gamma radiation sensitivity of Fen1 null mutant mice at the blastocyst stage. Mol Cell Biol 23:5346-5353.
Larsson C, Koch J, Nygren A, Janssen G, Raap A K, Landegren U, Nilsson M. 2004. In situ genotyping individual DNA molecules by target-primed rolling-circle amplification of padlock probes. Nat Methods 1:227-232.
Levsky J M, Singer R H. 2003. Fluorescence in situ hybridization: past, present and future. J Cell Sci 116:2833-2838.
Li X, Li J, Harrington J, Lieber M R, Burgers P M. 1995. Lagging strand DNA synthesis at the eukaryotic replication fork involves binding and stimulation of FEN-1 by proliferating cell nuclear antigen. J Biol Chem 270:22109-22112.
Lindahl T. 1993. Instability and decay of the primary structure of DNA. Nature 362:709-715.
Lisby M, Olesen J R, Skouboe C, Krogh B O, Straub T, Boege F, Velmurugan S, Martensen P M, Andersen A H, Jayaram M, Westergaard O, Knudsen B R. 2001. Residues within the N-terminal domain of human topoisomerase I play a direct role in relaxation. J Biol Chem 276:20220-20227.
Liu Y, Kao H I, Bambara R A. 2004. Flap endonuclease 1: a central component of DNA metabolism. Annu Rev Biochem 73:589-615.
Mueller R E, Parkes R K, Andrulis I, O'Malley F P. 2004. Amplification of the TOP2A gene does not predict high levels of topoisomerase II alpha protein in human breast tumor samples. Genes Chromosomes Cancer 39:288-297.
Murante R S, Rumbaugh J A, Barnes C J, Norton J R, Bambara R A. 1996. Calf RTH-1 nuclease can remove the initiator RNAs of Okazaki fragments by endonuclease activity. J Biol Chem 271:25888-25897.
Murante R S, Rust L, Bambara R A. 1995. Calf 5′ to 3′ exo/endonuclease must slide from a 5′ end of the substrate to perform structure-specific cleavage. J Biol Chem 270:30377-30383.
Nilsson M, Malmgren H, Samiotaki M, Kwiatkowski M, Chowdhary B P, Landegren U. 1994. Padlock probes: circularizing oligonucleotides for localized DNA detection. Science 265:2085-2088.
Pommier Y. 2006. Topoisomerase I inhibitors: camptothecins and beyond. Nat Rev Cancer 6:789-802.
Qiu J, Qian Y, Frank P, Wintersberger U, Shen B. 1999. Saccharomyces cerevisiae RNase H(35) functions in RNA primer removal during lagging-strand DNA synthesis, most efficiently in cooperation with Rad27 nuclease. Mol Cell Biol 19:8361-8371.
Soderberg O, Gullberg M, Jarvius M, Ridderstrale K, Leuchowius K J, Jarvius J, Wester K, Hydbring P, Bahram F, Larsson L G, Landegren U. 2006. Direct observation of individual endogenous protein complexes in situ by proximity ligation. Nat Methods 3:995-1000.
Warbrick E, Coates P J, Hall P A. 1998. Fen1 expression: a novel marker for cell proliferation. J Pathol 186:319-324.
Zheng L, Dai H, Qiu J, Huang Q, Shen B. 2007. Disruption of the FEN-1/PCNA interaction results in DNA replication defects, pulmonary hypoplasia, pancytopenia, and newborn lethality in mice. Mol Cell Biol.

### EXAMPLES

### Example 1

### Methods

### Oligonucleotides

Oligonucleotides (listed below) were purchased from DNA Technology A/S, Aarhus, Denmark.

| | |
|---|---|
| Name | Sequence |

| | |
|---|---|
| Fen1 | 5′-BIOTIN-GATATCGAAT TCCACTGTGA AGATCGCTTA TGGAATTCGA |
| | TATCAAGCCC TCAATGCACA TGTTTGGCTC CGCTTGATAT-3′ |
| Fen1 | 5′-P-CGAATTCC ACTGTGAAGATCGCTTAT GGAATTCGA |
| POS | TATCAAGC CCTCAATGCACATGTTTGGCTCC GCTTGATAT-3′ |
| Topo I | 5′-AGAAAAATTT TTAAAAAAAC TGTGAAGATC GCTTATTTTT TTAAAAATTT |
| | TTCTAAGTCT TTTAGATCCC TCAATGCTGC TGCTGTACTA CGATCTAAAA |
| | GACTTAGA-AMIN-3′ |
| Topo I | 5′-P-AGAAAAATTT TTAAAAAAAC TGTGAAGATC GCTTATTTTT TTAAAAATTT |
| POS | TTCTAAGTCT TTTAGATCCC TCAATGCTGC TGCTGTACTA CGATCTAAAAA CTT- |
| | 3′ |
| Primer1 | 5′-AMIN-CCAACCAACCAACCAA-ATAAGCGATCTTCACAGT-3′ |
| Primer2 | 5′-ATAAGCGATC TTCACAG-3′ |
| ID 16 | 5′-TAMRA-CCTCAATGCT GCTGCTGTAC TAC-3′ |
| ID 33 | 5′-FITC-CCTCAATGCA CATGTTTGGC TCC-3′ |

### Fen1 Reaction

### PAGE:

The Fen1 reaction was carried out in a mixture containing 40 mM Tris-HCl, pH 7.5, 10 mM MgCl₂, 1 mM DTT, 0.2 μg/μl BSA, 0.1 μM probe, 30 pg/μl (1 fmol/μl) of Fen1 enzyme (Alexis Biochemicals), and 0.03 u/μl of T4 DNA ligase (Fermentas) for 15 min at 30° C. The reaction was inactivated for 5 min at 95° C. For exonuclease digestion the reactions were supplemented with 7 units exonuclease I (Fermentas) and 70 units exonuclease III (Fermentas) and incubated for 60 min at 37° C. and inactivated for 15 min at 80° C. The reaction products were separated by 12% PAGE and stained with SYBR Gold (Molecular Probes).

### Solid Support Amplification:

5′-amine coupled primers were linked to CodeLink Activated Slides (Amersham Biosciences) according to the manufactures description. The enzymatic reactions were performed as described above. After enzyme inactivation the NaCl concentration was adjusted to 0.5 M before hybridization to the covalently coupled primer for 30 min at 37° C. Slides were washed in 0.1 M Tris-HCL, 150 mM NaCl and 0.3% SDS (washbuffer 1) for 2 min at room temperature followed by a wash in 0.1 M Tris-HCL, 150 mM NaCl and 0.05% Tween-20 (washbuffer 2). RCR was performed for 30 min at 37° C. in 1×Phi29 buffer supplemented with 0.2 μg/μl BSA, 250 μM dNTP, 5% glycerol, and 1 u/μl Phi29 DNA polymerase. The reaction was stopped by washing in washbuffer 1 and 2. RCPs were detected by hybridizing 0.17 μM of each of the detection oligonucleotides ID16 and ID33 in a buffer containing 20% formamide, 2×SSC and 5% glycerol for 30 min at 37° C. The slides were washed in washbuffer 1 and 2, dehydrated and mounted with Vectashield (Vector Laboratories).

### In Situ Detection:

Cells or tissue were thawed at room temperature and washed in 1×PBS (supplemented with DTT and PMSF) at 4° C. for three min. Cells were incubated with a mixture containing 40 mM Tris-HCl, pH 7.5, 10 mM MgCl₂, 1 mM DTT, 0.2 μg/μl BSA, 0.1 μM Fen1 probe for 30 min at 37° C. The reaction was supplemented with 0.5 mM ATP when indicated. The reaction was quenched by incubating 10 min in 70% ice-cold ethanol followed by dehydration. Rolling circle and detection was performed as described for the solid support assay, with the difference that the rolling circle reaction was supplemented with 0.02 μM primer and only washbuffer 2 was used.

### Topoisomerase I Reaction

### PAGE:

The topoisomerase I reaction was carried out in a mixture containing 10 mM Tris-HCl, pH 7.5, 5 mM CaCl₂, 5 mM MgCl₂, 10 mM DTT, 0.2 μg/μl BSA, 0.2 μM probe, 0.5 μl topoisomerase I for 60 min at 37° C. The reaction was inactivated for 5 min at 95° C. For exonuclease digestion the reactions were supplemented with 7 units exonuclease I (Fermentas) and 70 units exonuclease III (Fermentas) and incubated for 60 min at 37° C. and inactivated for 15 min at 80° C. The reaction products were separated by 12% PAGE and stained with SYBR Gold (Molecular Probes).

### Solid Support Amplification:

The topoisomerase I reaction was carried out in a mixture containing 10 mM Tris-HCl, pH 7.5, 5 mM CaCl₂, 5 mM MgCl₂, 10 mM DTT, 0.2 μg/μl BSA, 0.1 μM probe, 0.5 μl topoisomerase I for 60 min at 37° C. The reaction was inactivated for 5 min at 95° C. NaCl adjustment, hybridization, amplification and detection were performed as described for the Fen1 assay.

### In Situ Detection:

Cells or tissue were thawed at room temperature and washed in 1×PBS (supplemented with DTT and PMSF) at 4° C. for three min. Cells were incubated with a mixture containing 10 mM Tris-HCl, pH 7.5, 5 mM CaCl₂, 5 mM MgCl₂, 10 mM DTT, 0.2 μg/μl BSA, 0.1 μM Topo I probe for 60 min at 37° C. The reaction was supplemented with 0.5 mM ATP when indicated. The reaction was quenched by incubating 10 min in 70% ice-cold ethanol followed by dehydration. Rolling circle and detection was performed as described for the Fen1 in situ assay.

### Cells Lines

SiHa cells (30 μl with a density of 50,000 cells/ml (in MEM media+10% FCS)) were grown for 48-72 hours in teflon-printed diagnostic well slides (5 mm) (Immuno-Cell Int.), washed in 1×PBS, covered with Tissue-Tek (Sakura) and snap-frozen on a metal-block cooled on dry ice. Cells were stored at −80° C. until use.

### Tissue Sections

Frozen anonymous breast cancer tissue was cut on a microtone (4 μm sections) and placed on Superfrost slides (Menzel-Glaser).

### Image Analysis

Both solid support and cell slides were analyzed with a Leica epifluorescence microscope and images were recorded with a SenSys CCD-camera operated by the SmartCapture 2 version 2.0 from Digitalscientific (Cambridge, UK). A 63× objective (Leica) was used for all images. Thresholding was performed using Adobe Photoshop (Adobe Systems).

### Example 2

### Gel-Based Detection Assays

### Fen 1:

The experimental setup of the Fen1 activity assay was confirmed by PAGE (FIG. 2). The linear probe was degradable using a combination of exonuclease I and III (both exonucleases were used because the probe contained both single and double stranded regions) (compare FIG. 2, lane 1 and 2). The probe was also degradable when Fen1 and T4 DNA ligase were added separately (compare FIG. 2, lane 3 and 4 and lane 5 and 6). In contrast, when both Fen1 and T4 DNA ligase were included, a faster migrating product, resistant to exonucleases, appeared (compare FIG. 2 lane 7 and 8), indicating that the probe had been circularized. This circular product had the same migration speed as a circularized control probe with the same sequence as the Fen1 modified product (compare FIG. 2, lane 8 and 12). When only Fen1 was applied to the reaction, the linear product had a faster migration than without Fen1 (compare FIG. 2, lane 1 and 5), likely resulting from Fen1 removing the four nucleotide overhang present in the probe (see Table 1). Thus, the probe was indeed a substrate for Fen1 and the resulting product of the Fen1 modification was a substrate for T4 DNA ligase, which was able to circularize the modified probe.

### Topoisomerase 1

To verify that the Topo I probe was a substrate for topoisomerase I, a gel-based assay was employed (FIG. 3). Incubation of the probe with topoisomerase I gave rise to a product resistant to exonuclease digestion and with a slower mobility in the gel than the linear substrate (compare FIG. 3, lane 5 and 8). This product had the same migration speed as a circularized control probe with the same sequence (compare FIG. 3, lane 8 and 12). Furthermore, phosphorylation of the probe made it resistant to topoisomerase I catalysis, as expected according to the catalytic mechanism of topoisomerase I (Champoux, 2001), resulting in degradation by exonuclease treatment (compare FIG. 3, lane 4 and 8). Thus, the probe was indeed recognized and circularized by topoisomerase I. The circularized Topo I probe had a slower migration speed than the corresponding linear probe, whereas the circularized Fen1 probe had a faster migration speed than the corresponding linear probe. Variable shifting of circularized oligonucleotides has also been observed in our laboratory with other probes containing double stranded regions.

### Example 3

### Solid Support Amplification Assays

To verify that the resulting products were indeed circularized, solid support assays comprising RCR and fluorescent labeling were designed.

### Fen1

Products corresponding to lane 1, 3, 5 and 7 from FIG. 2 were hybridized to a surface coated with 5′-amin-coupled oligonucleotides (primer 1). Following hybridization the covalently coupled oligonucleotide functioned as a primer for RCR. After amplification the products were detected by hybridizing a labeled oligonucleotide to the RCP. As expected, according to the gel-based assay, RCP's could only be detected when both Fen1 and ligase were present in the reaction mixture (FIG. 4). Since each Fen1 enzyme can prepare several probes for ligation, this assay does not correspond to single-molecule detection of Fen1 enzymes, but instead each green dot corresponds to a single circularization event (if two or more RCR reactions have not taken place in close proximity on the solid support, thereby giving rise to a dot corresponding to several circularization events).

### Topoisomerase I

To verify the circularization of the Topo I probe, a solid support assay, similar to the Fen1 assay, was performed. Signals only appeared when topoisomerase I was present (compare FIGS. 5, C and D), indicating circularization of the probe. Furthermore, when the probe was 5′-phosphorylated virtually no signals were seen, indicating that the ligation event was prevented, (compare FIGS. 5, B and D). The very few signals which appear are most likely caused by un-phosphorylated probes, indicating that the phosphorylation was incomplete (FIG. 5B).

### Example 4

### Assays for Direct Detection in Cells and Tissue Samples

Monitoring protein activity on single cells would be a strong molecular tool. Before our method could be implemented to in situ assays, several factors had to be considered. I) The probe should not hybridize to a target nucleic acid sequence like a standard FISH probe does, and it should not bind strongly to a protein like an antibody does. II) Fixation (ethanol and paraformaldehyde) seems to inactivate the enzymes in question. III) Since we would like to detect protein activity, cell penetration would not be an option.

A protocol for adherent cell lines was developed. We were able to maintain protein activity in frozen cells by using Tissue-Tek, which preserves cell morphology and is water dissolvable making it easy to remove by washing. Furthermore, with the probes used we were able to detect protein activity for several months after freezing (when stored at −80° C.).

Following probe incubation the mixture was quenched in 70% ice-cold ethanol. This step traps a number of the probes to the slide, but since only circularized probes can be visualized by RCR, trapped linear probes should not give any background staining. Some signals were likely lost in this protocol, since the probes did not bind to a target in the cells. This was confirmed by the observation that the probe mix, following cell incubation, could be transferred to a solid support assay generating specific signals there (data not shown). Furthermore, in situ signals were not located specifically inside cells. This indicates that the lack of fixation allowed proteins to diffuse out of the cells. This was confirmed by applying a reaction mixture to the cells (leaving out the probe), and following incubation the mixture could be transferred to a solid support with a pre-hybridized linear probe and subsequently obtaining specific signals there (data not shown). The current protocol may thus predominantly be seen as an alternative to making cell extracts by time consuming procedures, as well as a method to examine only a few cells in each reaction.

### Fen 1 Probe

The Fen1 probe only gave rise to signals in the presence of ATP, indicating that following cellular cleavage, an ATP-dependent cellular ligase was sealing the strand-break (FIG. 6, A-B). Addition of dNTP did not stimulate the reaction (data not shown), indicating that the repair event was independent of polymerase activity. Most of the visible signals were present outside the nucleus, though signals could be seen in the nucleus if other focal planes were chosen. Fen1 is believed to be almost exclusively located in the nucleus (Warbrick et al., 1998) indicating that the signals in the cytosol are an artefact of the method. The low amount of signals in the nucleus could be caused by competition from cellular DNA, whereas enzymes which have escaped from the nucleus did not have any competing cellular DNA. This is in agreement with the observation that carrier DNA strongly inhibited the reaction (data not shown). To test the system on tissue, frozen anonymous breast cancer tissue was tested for protein activity with the Fen1 probe; many signals were detected in an ATP-dependent manner (FIG. 6, C-D). Like the results from the cell line, the signals were not located specifically inside cells.

### Topo I Probe

Signals appeared also with the topoisomerase I assay on cells. Unlike the Fen1 assay, the signals were ATP-independent indicating a one-enzyme ligase-independent reaction (FIG. 6, E-F). On the tissue sections, signals could likewise be obtained in an ATP-independent manner (FIG. 6, G-H).

Taken together, these results indicate that protein activity can indeed be detected in cell and tissue preparations at single molecule resolution. Fen1 is just one of many enzymes involved in DNA repair, similar assays could be developed to detect the activity of different DNA modifying enzymes, e.g. glycosylases which recognize different DNA damages (such as methylations, 8-hydroxy-2′-deoxyguanosines (8-oxo-dG), etc), APE1 which recognizes abasic sites, methylases, recombinases or whole enzymatic pathways. Such assays could in theory be multiplexed allowing for e.g. the evaluation of the repair-state of a single cell or a population of cells, by using an arsenal of synthetic oligonucleotides containing different DNA-lesions; several modified nucleotides, corresponding to cellular DNA-lesions, are now commercially available (Iwai, 2006). Likewise, designing specific probes for different types of topoisomerases could be a strong tool when the type chemotherapy should be chosen in cancer treatments, since topoisomerases are targets for several chemotherapeutics (Pommier, 2006). Optimization of probes and protocols for such assays are currently in progress in our laboratory. In the future, multiplexed assays for detection of the activity of different enzymes (e.g. the repair of a large panel of DNA damages) analyzed at single cell resolution, would be a strong tool not only in basic research but also in cancer prognostics/diagnostics.

1. A method for determining in a biological sample either a) the presence of one or more enzyme activities involved in circularising a non-circular oligonucleotide probe, or b) the absence of at least one such enzyme activity in said biological sample, said method comprising the steps of
i) providing a biological sample to be analysed for the presence or absence of at least one enzyme activity,
ii) providing an oligonucleotide probe comprising an unprocessed substrate moiety capable of being processed by at least one of said one or more enzymes,
wherein said oligonucleotide probe comprises a single strand of contiguous nucleotides or a plurality of single strands of contiguous nucleotides capable of hybridisation to each other,
wherein said oligonucleotide probe comprising an unprocessed substrate moiety cannot be amplified by rolling circle replication in the absence of said processing,
iii) contacting the biological sample with the oligonucleotide probe under conditions allowing said one or more enzymes, if present in said biological sample, to act on the substrate moiety,
wherein said action results in the processing of the substrate moiety and the formation of a circular, oligonucleotide template capable of being amplified by rolling circle replication,
iv) amplifying the circular oligonucleotide template, when such a template is formed in step iii), by using a polymerase capable of performing multiple rounds of rolling circle replication of said circular oligonucleotide template, optionally by initially contacting said circular oligonucleotide template with a suitable primer, and generating a rolling circle amplification product comprising multiple copies of the circular oligonucleotide template, or
v) generating no rolling circle amplification product when no circular oligonucleotide template is formed in step iii) as a result of said one or more enzyme activities not being present in said biological sample,
wherein steps iv) and v) are mutually exclusive,
wherein said amplification product is indicative of the presence in said biological sample of said one or more enzyme activities involved in circularising a non-circular oligonucleotide probe,
and wherein no amplification product is formed in the absence of at least one such enzyme activity in said biological sample.

2. The method of claim 1, wherein said oligonucleotide probe further comprises one or more non-hybridised, single stranded portion(s) and one or more double stranded portion(s), each double stranded portion comprising complementary nucleotide strands.

3. The method of claim 2, wherein said one or more single stranded portion(s) of said oligonucleotide probe does not hybridise to a complementary nucleotide sequence.

4. The method of claim 2, wherein said oligonucleotide probe comprises at least one nucleotide sequence which is complementary to one or more of said single stranded portion(s) of said oligonucleotide probe.

5. The method of any of claims 1 to 4, wherein said oligonucleotide probe is in the form of a single oligonucleotide comprising a contiguous sequence of nucleotides, wherein at least some of said nucleotides are capable of forming a double stranded sequence comprising complementary nucleotide strands.

6. The method of any of claims 1 to 4, wherein said oligonucleotide probe comprises more than one single oligonucleotide, wherein each oligonucleotide of the probe comprises a single contiguous sequence of nucleotides, wherein at least some of said nucleotides of the different oligonucleotides of the probe are capable of hybridising to each other.

7. The method of any of claims 1 to 6, wherein the probe is a self-templating probe comprising at least two double stranded portions each comprising complementary nucleotide strands separated at the proximal ends by an unprocessed substrate moiety.

8. The method of claim 7, wherein the at least two double stranded portions comprising complementary nucleotide strands are each joined at the distal ends by a single stranded nucleotide forming a loop structure.

9. The method of any of claims 1 to 8, wherein the unprocessed substrate moiety comprises a nick or a single stranded nucleotide region.

10. The method of claim 9, wherein the single stranded nucleotide region is adjoined at both ends to a double stranded nucleotide region.

11. The method of claim 9, wherein the single stranded nucleotide region is a 5′ overhang nucleotide region adjoined at one end to a double stranded nucleotide region of the oligonucleotide probe.

12. The method of claim 9, wherein the single stranded nucleotide region is a 3′ overhang nucleotide region adjoined at one end to a double stranded nucleotide region of the oligonucleotide probe.

13. The method of any of claims 9 to 12, wherein the single stranded nucleotide region preferably contains less than 20 nucleotides.

14. The method of any of claims 9 to 12, wherein the single stranded nucleotide region preferably contains less than 15 nucleotides.

15. The method of any of claims 9 to 12, wherein the single stranded nucleotide region preferably contains less than 10 nucleotides.

16. The method of any of claims 9 to 12, wherein the single stranded nucleotide region preferably contains less than 5 nucleotides.

17. The method of any of claims 9 to 12, wherein the single stranded nucleotide region preferably contains less than 3 nucleotides.

18. The method of any of claims 1 to 17, wherein the substrate moiety conversion is mediated specifically by a flap endonuclease activity present in said sample in combination with a ligase activity present in said sample and/or added to said sample.

19. The method of claim 18, wherein the flap endonuclease activity is mediated by FEN1, DNA2P or EXO1.

20. The method of any of claims 1 to 17, wherein the substrate moiety conversion is mediated specifically by a topoisomerase activity present in said sample.

21. The method of claim 20, wherein the topoisomerase activity is mediated by a Topoisomerase I.

22. The method of claim 20, wherein the topoisomerase activity is mediated by a Topoisomerase II.

23. The method of any of claims 1 to 17, wherein said unprocessed substrate moiety is selected from the group consisting of
i) unprocessed substrate moieties comprising or consisting of one or more nick(s) in one or more single strand(s) of a double stranded nucleotide sequence of said oligonucleotide probe, said one or more nick(s) forming one or more unprocessed substrate moieties of said oligonucleotide probe,
ii) unprocessed substrate moieties comprising or consisting of one or more single stranded nucleotide sequence(s) joined at one or both ends thereof by a double stranded nucleotide sequence, said single stranded sequence(s) creating one or more gap structure(s) forming one or more unprocessed substrate moieties of said oligonucleotide probe, and
iii) unprocessed substrate moieties comprising or consisting of one or more nick(s) or one or more gap(s), said gap(s) being in the form of a single stranded nucleotide sequence, said nick(s) or gap(s) being joined at one end thereof to a double stranded nucleotide sequence and at the other end thereof to at least one single stranded overhang joined to a double stranded nucleotide sequence of said oligonucleotide probe, wherein said nick(s) or gap(s) in combination with the at least one single stranded overhang forms one or more unprocessed substrate moieties of said oligonucleotide probe.

24. The method of claim 23, wherein said unprocessed substrate moiety comprises or consists of one or more nick(s) in one or more single strand(s) of a double stranded nucleotide sequence of said oligonucleotide probe, said one or more nick(s) forming one or more unprocessed substrate moieties of said oligonucleotide probe.

25. The method of claim 24, wherein said one or more enzyme activities present in said sample comprises a ligase activity capable of ligating said nick of said oligonucleotide probe.

26. The method of any of claims 24 and 25, wherein a circular oligonucleotide template capable of being amplified by rolling circle amplification is generated by ligating said nick, said ligation being performed by at least one ligase activity present in said sample.

27. The method of claim 26, wherein said circular oligonucleotide template is amplified by rolling circle amplification, said amplification being indicative of the presence in said sample of at least one ligase activity.

28. The method of claim 27, wherein said rolling circle amplification product is detected by detecting a label covalently or non-covalently associated with said rolling circle amplification product, wherein said label is preferably fluorescently detectable, wherein said label is either a fluorescent molecule incorporated into the rolling circle amplification product, for example by being present in the primer used for probe amplification and generation of the rolling circle amplification product, or by being linked to a nucleotide incorporated into the rolling circle amplification product during the probe amplification process, or by being linked to a fluorescently labelled oligonucleotide hybridising to the rolling circle amplification product, or wherein said label is a molecule or a chemical group which can be detected by a fluorescently labelled molecule, such as an antibody.

29. The method of claim 23, wherein said unprocessed substrate moiety comprises or consists of one or more single stranded nucleotide sequence(s) joined at one or both ends by a double stranded nucleotide sequence, said single stranded sequence(s) creating one or more gap structure(s) forming one or more unprocessed substrate moieties of said oligonucleotide probe.

30. The method of claim 29, wherein a circular, oligonucleotide template capable of being amplified by rolling circle amplification is generated through a) filling-in said gap by using the at least one enzyme activity present in said sample which is capable of performing a template directed nucleotide extension reaction, and b) ligating one or both of the end-positioned, filled-in nucleotides to the remaining, double stranded part of the oligonucleotide probe.

31. The method of claim 30, wherein said circular, oligonucleotide template is amplified by rolling circle amplification, said amplification being indicative of the presence in said sample of at least one enzyme activity capable of performing template directed nucleotide extension and/or nucleotide ligation.

32. The method of claim 31, wherein said rolling circle amplification product is detected by detecting a label covalently or non-covalently associated with said rolling circle amplification product, wherein said label is preferably fluorescently detectable, wherein said label is either a fluorescent molecule incorporated into the rolling circle amplification product, for example by being present in the primer used for probe amplification and generation of the rolling circle amplification product, or by being linked to a nucleotide incorporated into the rolling circle amplification product during the probe amplification process, or by being linked to a fluorescently labelled oligonucleotide hybridising to the rolling circle amplification product, or wherein said label is a molecule or a chemical group which can be detected by a fluorescently labelled molecule, such as an antibody.

33. The method of claim 23, wherein said substrate moiety comprises or consists of one or more nick(s) and/or one or more gap(s), said gap(s) being in the form of a single stranded nucleotide sequence, said nick(s) or gap(s) being joined at one end to a double stranded nucleotide sequence and at the other end to at least one single stranded overhang joined to a double stranded nucleotide sequence of said oligonucleotide probe, wherein said nick(s) or gap(s) in combination with the at least one single stranded overhang forms one or more unprocessed substrate moieties of said oligonucleotide probe.

34. The method of claim 33, wherein the one or more overhang(s) is a 5′ overhang, said oligonucleotide probe further comprising at least one 3′ end.

35. The method of claim 34, wherein the 5′ overhang is protected by a protection group preventing an exonuclease from digesting the 5′ overhang.

36. The method of claim 35, wherein a circular, oligonucleotide template capable of being amplified by rolling circle amplification is generated by a) endonucleolytic digestion of said 5′ overhang and b) ligation of the end of the nucleotide strand resulting from the endonucleolytic digestion to a nucleotide strand of the remaining part of the oligonucleotide probe.

37. The method of claim 36, wherein said circular, oligonucleotide template is amplified by rolling circle amplification, said amplification being indicative of the presence in said sample of at least one endonuclease.

38. The method of claim 37, wherein said rolling circle amplification product is detected by detecting a label covalently or non-covalently associated with said rolling circle amplification product, wherein said label is preferably fluorescently detectable, wherein said label is either a fluorescent molecule incorporated into the rolling circle amplification product, for example by being present in the primer used for probe amplification and generation of the rolling circle amplification product, or by being linked to a nucleotide incorporated into the rolling circle amplification product during the probe amplification process, or by being linked to a fluorescently labelled oligonucleotide hybridising to the rolling circle amplification product, or wherein said label is a molecule or a chemical group which can be detected by a fluorescently labelled molecule, such as an antibody.

39. The method of any of claims 34 and 35, wherein the 5′ end of the 5′ overhang comprises a protection group in the form of a phosphate group or different from a phosphate group, wherein said protection group prevents ligation of said 5′ overhang to a 3′ end of a strand of the remaining part of the oligonucleotide probe.

40. The method of claim 39, wherein said protection group different from a phosphate group is selected from the group consisting of H, biotin, amin, and an optionally substituted C₁-C₆-linker.

41. The method of any of claims 39 and 40, wherein a topoisomerase I activity present in said sample cannot process the unprocessed substrate moiety of said oligonucleotide probe and generate a circular oligonucleotide template.

42. The method of claim 41, wherein a flap endonuclease activity present in said sample processes the unprocessed substrate moiety of said oligonucleotide probe, said processing resulting in the formation of a 5′ end having a phosphate reactive group capable of being ligated with a 3′ end of a strand of the remaining part of the oligonucleotide probe, thereby generating a circular oligonucleotide template capable of being amplified by rolling circle amplification.

43. The method of claim 42, wherein said circular oligonucleotide template generated by the flap endonuclease activity and a ligase activity present in said sample is amplified by rolling circle amplification, thereby generating a rolling circle amplification product.

44. The method of claim 43, wherein said rolling circle amplification product is indicative of the presence in said sample of a flap endonuclease activity and a ligase activity.

45. The method of any of claims 34 and 35, wherein the 3′ end of the oligonucleotide probe comprises a protection group different from a hydroxy group, wherein said protection group prevents ligation of said 5′ overhang to the 3′ end of a strand of the remaining part of the oligonucleotide probe.

46. The method of claim 45, wherein said protection group different from a phosphate group is selected from the group consisting of H, biotin, amin, and an optionally substituted C₁-C₆-linker.

47. The method of any of claims 34 and 35, wherein a flap endonuclease activity present in said sample cannot process the unprocessed substrate moiety of said oligonucleotide probe and provide an oligonucleotide which can be ligated by a ligase to generate a circular oligonucleotide template.

48. The method of claim 47, wherein a topoisomerase I activity present in said sample processes the unprocessed substrate moiety of said oligonucleotide probe, said processing resulting in the formation of a 3′-phospho-tyrosine intermediate, in the form of a covalent DNA-protein intermediate, capable of being ligated with the HO-group of the 5′-end of the 5′-overhang of the oligonucleotide probe, wherein said ligation results in the formation of a circular oligonucleotide template capable of being amplified by rolling circle amplification.

49. The method of claim 48, wherein said circular oligonucleotide template generated by the topoisomerase I activity present in said sample is amplified by rolling circle amplification, thereby generating a rolling circle amplification product.

50. The method of claim 49, wherein said rolling circle amplification product is indicative of the presence in said sample of a topoisomerase I activity.

51. The method of any of claims 34 and 35, wherein the nucleotides of the 5′ overhang each comprises a nucleobase and a backbone unit, wherein the backbone unit comprises a sugar moiety and an internucleoside linker.

52. The method of claim 51, wherein the nucleobase of the nucleotides of the 5′ overhang are selected from naturally occurring nucleobases and non-naturally occurring nucleobases.

53. The method of claim 51, wherein the backbone unit of neighbouring nucleobases is selected from naturally occurring backbone units and non-naturally occurring backbone units.

54. The method of claim 51, wherein the sugar moiety of the backbone unit of neighbouring nucleobases is selected from naturally occurring sugar moieties and non-naturally occurring sugar moieties.

55. The method of claim 51, wherein the internucleoside linker of the backbone unit of neighbouring nucleobases is selected from naturally occurring internucleoside linkers and non-naturally occurring internucleoside linkers.

56. The method of claim 52, wherein the nucleobases of the 5′ overhang are selected independently from the group consisting of natural and non-natural purine heterocycles, natural and non-natural pyrimidine heterocycles, including heterocyclic, non-natural analogues and tautomers of said natural purine heterocycles and said natural pyrimidine heterocycles.

57. The method of claim 52, wherein the nucleobases of the 5′ overhang are selected independently from the group consisting of adenine, guanine, isoguanine, thymine, cytosine, isocytosine, pseudoisocytosine, uracil, inosine, purine, xanthine, diaminopurine, 8-oxo-N⁶-methyladenine, 7-deazaxanthine, 7-deazaguanine, N⁴,N⁴-ethanocytosin, N⁶,N⁶-ethano-2,6-diamino-purine, 5-methylcytosine, 5-(C³—C⁶)-alkynylcytosine, 5-fluorouracil, 5-bromouracil and 2-hydroxy-5-methyl-4-triazolopyridine.

58. The method of claim 52, wherein the nucleobases of the 5′ overhang are selected independently from the group consisting of adenine, guanine, thymine, cytosine, 5-methylcytosine and uracil.

59. The method of claim 53, wherein the backbone units of the nucleotides of the 5′ overhang are the same or different backbone units.

60. The method of claim 59, wherein the same or different backbone units of the nucleotides of the 5′ overhang are selected independently from the group consisting of 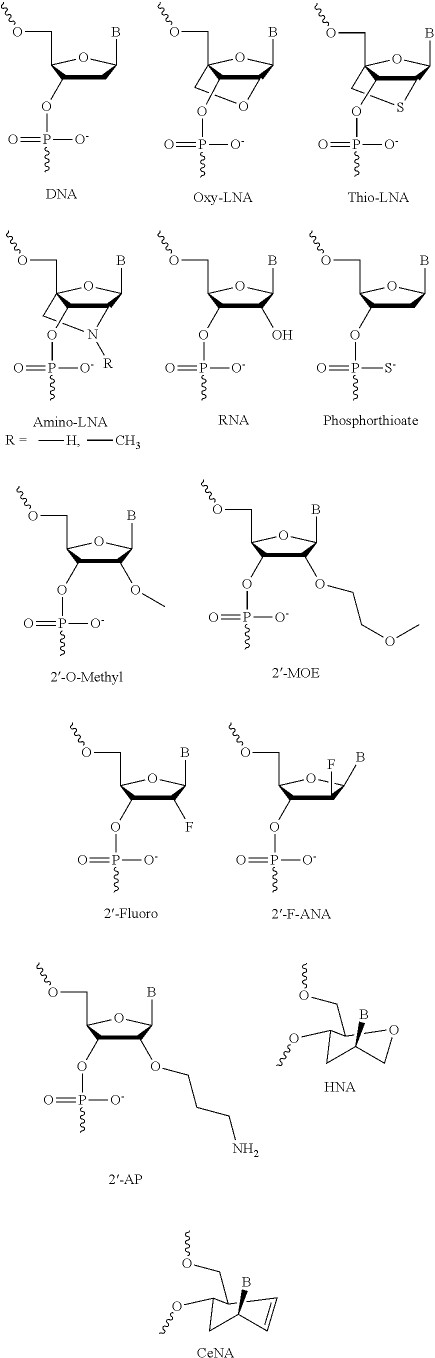 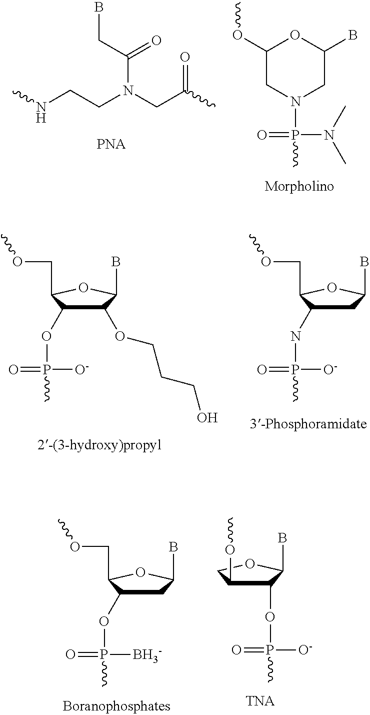
wherein B denotes a nucleobase.

61. The method of claim 54, wherein the sugar moiety of the backbone unit of the nucleotides of the 5′ overhang comprises or consists of a pentose.

62. The method of claim 61, wherein the pentose is selected from the group consisting of ribose, 2′-deoxyribose, 2′-O-methyl-ribose, 2′-fluor-ribose, and 2′-4′-O-methylene-ribose (LNA).

63. The method of any of claims 61 and 62, wherein the nucleobase of the nucleotide is attached to the 1′ position of the pentose.

64. The method of claim 63, wherein the backbone units linking any two neighbouring nucleotides of the 5′ overhang are the same or different backbone units.

65. The method of claim 64, wherein at least some of the nucleotides of the 5′ overhang are linked by different backbone units.

66. The method of claim 65, wherein at least some of said different backbone units are non-natural backbone units.

67. The method of claim 55, wherein the internucleoside linkers linking any two neighbouring nucleotides of the 5′ overhang are the same or different internucleoside linkers.

68. The method of claim 55, wherein at least some of the nucleotides of the 5′ overhang are linked by different internucleoside linkers.

69. The method of claim 68, wherein at least some of said different internucleotide linkers are non-natural internucleotide linkers.

70. The method of claim 55, wherein the internucleoside linkers of the 5′ overhang are selected from the group consisting of phosphodiester bonds, phosphorothioate bonds, methylphosphonate bonds, phosphoramidate bonds, phosphotriester bonds and phosphodithioate bonds.

71. The method of claim 55, wherein the internucleoside linkers of the 5′ overhang are selected from the group consisting of phosphorothioate bonds, methylphosphonate bonds, phosphoramidate bonds, phosphotriester bonds and phosphodithioate bonds.

72. The method of any of claims 34 and 35, wherein the nucleotides of the 5′ overhang are selected from naturally occurring nucleosides of the DNA and RNA family connected through phosphodiester linkages and at least one non-natural nucleotide selected from the group consisting of nucleotides comprising a non-natural nucleobase and/or a non-natural backbone unit comprising a non-natural sugar moiety and/or a non-natural internucleoside linker.

73. The method of claim 72, wherein the naturally occurring nucleosides are deoxynucleosides selected from the group consisting of deoxyadenosine, deoxyguanosine, deoxythymidine, and deoxycytidine.

74. The method of claim 72, wherein the naturally occurring nucleosides are selected from the group of nucleotides consisting of adenosine, guanosine, uridine, cytidine, and inosine.

75. The method of any of claims 72 to 74, wherein the non-natural nucleobase of the one or more non-natural nucleotides is selected from the group consisting of 8-oxo-N⁶-methyladenine, 7-deazaxanthine, 7-deazaguanine, N⁴,N⁴-ethanocytosin, N⁶,N⁶-ethano-2,6-diamino-purine, 5-methylcytosine, 5-(C³—C⁶)-alkynylcytosine, 5-fluorouracil, 5-bromouracil, pseudoisocytosine, 2-hydroxy-5-methyl-4-triazolopyridine, isocytosine, isoguanine and inosine.

76. The method of any of claims 72 to 75, wherein the non-natural backbone unit of the one or more non-natural nucleotides is selected from the group consisting of 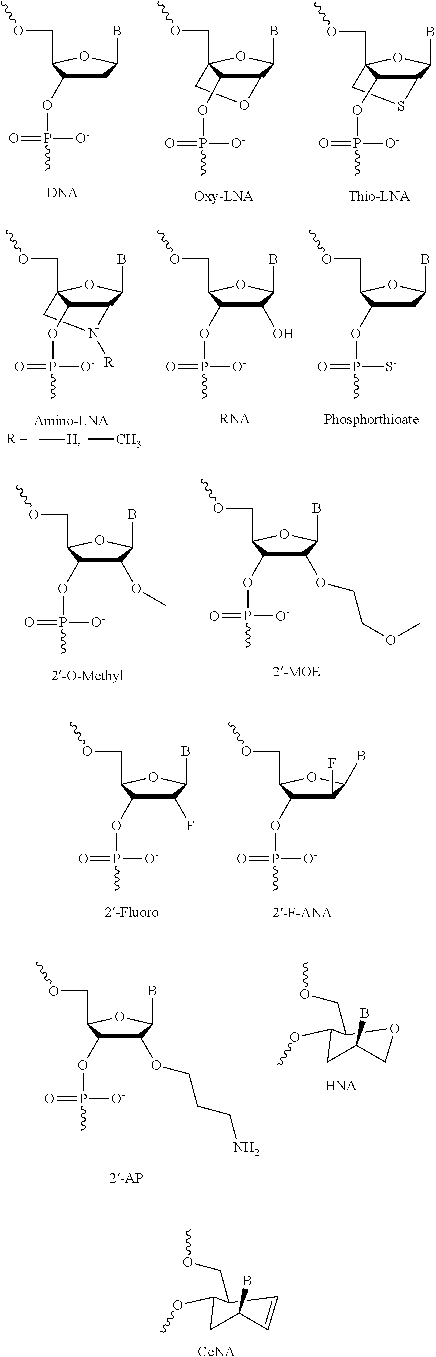 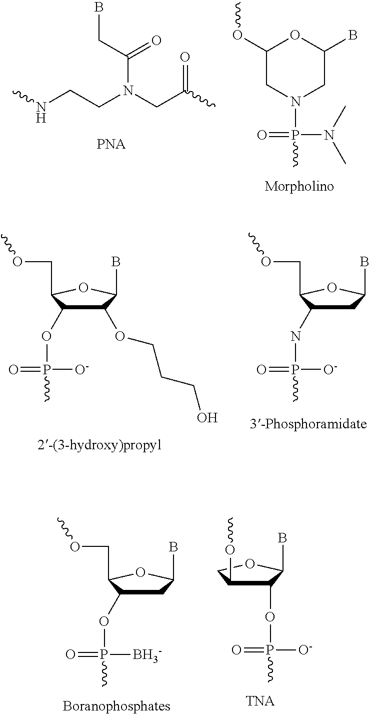
wherein B denotes a nucleobase.

77. The method of any of claims 72 to 76, wherein the non-natural sugar moiety of the one or more non-natural backbone unit(s) is selected from the group consisting of 2′-deoxyribose, 2′-O-methyl-ribose, 2′-fluor-ribose and 2′-4′-O-methylene-ribose (LNA).

78. The method of any of claims 72 to 77, wherein the non-natural internucleoside linker of the one or more non-natural backbone unit(s) is selected from the group consisting of phosphorothioate bonds, methylphosphonate bonds, phosphoramidate bonds, phosphotriester bonds and phosphodithioate bonds.

79. The method of claim 52, wherein said 5′ overhang comprises naturally occurring nucleobases connected by naturally occurring backbone units, wherein said naturally occurring nucleobases and said naturally occurring backbone units do not prevent exonuclease degradation of said 5′ overhang.

80. The method of claim 79, wherein said 5′ overhang further comprises non-naturally occurring nucleobases which do not prevent exonuclease degradation of said 5′ overhang.

81. The method of claim 80, wherein said non-naturally occurring backbone units comprising sugar moieties and internucleoside linkers do not prevent exonuclease degradation of said 5′ overhang.

82. The method of claim 81, wherein said sugar moieties are non-naturally occurring sugar moieties which do not prevent exonuclease degradation of said 5′ overhang.

83. The method of claim 81, wherein said internucleoside linkers are non-naturally occurring internucleoside linkers which do not prevent exonuclease degradation of said 5′ overhang.

84. The method of any of claims 79 to 83, wherein said one or more enzyme activities present in said sample comprises a 5′ to 3′ exonuclease activity capable of cleaving one or more, such as all of the internucleoside linkers connecting the nucleotides of the 5′ overhang and/or a ligase activity.

85. The method of claim 84, wherein the circular oligonucleotide template capable of being amplified by rolling circle amplification is generated by ligating the oligonucleotide probe comprising a substrate moiety processed by 5′ to 3′ exonucleolytically digestion of the 5′ overhang, said ligation being performed by at least one ligase activity present in said sample.

86. The method of claim 85, wherein said circular, oligonucleotide template is amplified by rolling circle amplification, said amplification being indicative of the presence in said sample of at least one 5′ to 3′ exonuclease activity and at least one ligase activity.

87. The method of claim 86, wherein said rolling circle amplification product is detected by detecting a label covalently or non-covalently associated with said rolling circle amplification product, wherein said label is preferably fluorescently detectable, wherein said label is either a fluorescent molecule incorporated into the rolling circle amplification product, for example by being present in the primer used for probe amplification and generation of the rolling circle amplification product, or by being linked to a nucleotide incorporated into the rolling circle amplification product during the probe amplification process, or by being linked to a fluorescently labelled oligonucleotide hybridising to the rolling circle amplification product, or wherein said label is a molecule or a chemical group which can be detected by a fluorescently labelled molecule, such as an antibody.

88. The method of claim 52, wherein said 5′ overhang comprises non-naturally occurring nucleobases connected by naturally occurring backbone units and/or non-naturally occurring backbone units, said backbone units comprising a sugar moiety and an internucleoside linker, wherein said non-naturally occurring nucleobases and said non-naturally occurring backbone units, when present, prevent exonuclease degradation of said 5′ overhang.

89. The method of claim 88, wherein said non-naturally occurring nucleobases alone prevents exonuclease degradation of said 5′ overhang.

90. The method of claim 88, wherein said non-naturally occurring backbone units prevent exonuclease degradation of said 5′ overhang.

91. The method of claim 88, wherein said non-naturally occurring sugar moieties prevent exonuclease degradation of said 5′ overhang.

92. The method of claim 88, wherein said non-naturally occurring internucleoside linkers prevent exonuclease degradation of said 5′ overhang.

93. The method of any of claims 88 to 92, wherein a 5′ to 3′ exonuclease activity present in said sample cannot cleave the internucleoside linkers connecting the nucleotides of the 5′ overhang.

94. The method of any of claims 88 to 92, wherein said one or more enzyme activities present in said sample further comprises a flap endonuclease activity capable of cleaving the internucleoside linkers connecting the nucleotides of the 5′ overhang and/or a ligase activity.

95. The method of claim 94, wherein a circular, oligonucleotide template capable of being amplified by rolling circle amplification is generated by ligating the oligonucleotide probe comprising a processed substrate moiety, wherein said substrate moiety processing comprises flap endonucleolytically cleaving at least one internucleoside linker of the 5′ overhang of the probe, thereby releasing the 5′ overhang from the remaining part of the oligonucleotide probe, wherein the ligation is performed by at least one ligase activity present in said sample.

96. The method of claim 95, wherein said circular, oligonucleotide template is amplified by rolling circle amplification, said amplification being indicative of the presence in said sample of at least one flap endonuclease activity and at least one ligase activity.

97. The method of claim 96, wherein said rolling circle amplification product is detected by detecting a label covalently or non-covalently associated with said rolling circle amplification product, wherein said label is preferably fluorescently detectable, wherein said label is either a fluorescent molecule incorporated into the rolling circle amplification product, for example by being present in the primer used for probe amplification and generation of the rolling circle amplification product, or by being linked to a nucleotide incorporated into the rolling circle amplification product during the probe amplification process, or by being linked to a fluorescently labelled oligonucleotide hybridising to the rolling circle amplification product, or wherein said label is a molecule or a chemical group which can be detected by a fluorescently labelled molecule, such as an antibody.

98. The method of claim 33, wherein the one or more overhang(s) is a 3′ overhang, said oligonucleotide probe further comprising at least one 5′ end.

99. The method of claim 98, wherein the 3′ overhang is protected by a protection group preventing an exonuclease from digesting the 3′ overhang.

100. The method of claim 99, wherein a circular, oligonucleotide template capable of being amplified by rolling circle amplification is generated by a) endonucleolytic digestion of said 3′ overhang and b) ligation of the end of the nucleotide strand resulting from the endonucleolytic digestion to a nucleotide strand of the remaining part of the oligonucleotide probe.

101. The method of claim 100, wherein said circular, oligonucleotide template is amplified by rolling circle amplification, said amplification being indicative of the presence in said sample of at least one endonuclease.

102. The method of claim 101, wherein said rolling circle amplification product is detected by detecting a label covalently or non-covalently associated with said rolling circle amplification product, wherein said label is preferably fluorescently detectable, wherein said label is either a fluorescent molecule incorporated into the rolling circle amplification product, for example by being present in the primer used for probe amplification and generation of the rolling circle amplification product, or by being linked to a nucleotide incorporated into the rolling circle amplification product during the probe amplification process, or by being linked to a fluorescently labelled oligonucleotide hybridising to the rolling circle amplification product, or wherein said label is a molecule or a chemical group which can be detected by a fluorescently labelled molecule, such as an antibody.

103. The method of any of claims 98 and 99, wherein a topoisomerase II activity present in said sample processes the unprocessed substrate moiety of said oligonucleotide probe and thereby provides a circular oligonucleotide template.

104. The method of claim 103, wherein said circular oligonucleotide template generated by the topoisomerase II activity present in said sample is amplified by rolling circle amplification, thereby generating a rolling circle amplification product.

105. The method of claim 104, wherein said rolling circle amplification product is indicative of the presence in said sample of a topoisomerase II activity.

106. The method of claim 105, wherein said rolling circle amplification product is detected by detecting a label covalently or non-covalently associated with said rolling circle amplification product, wherein said label is preferably fluorescently detectable, wherein said label is either a fluorescent molecule incorporated into the rolling circle amplification product, for example by being present in the primer used for probe amplification and generation of the rolling circle amplification product, or by being linked to a nucleotide incorporated into the rolling circle amplification product during the probe amplification process, or by being linked to a fluorescently labelled oligonucleotide hybridising to the rolling circle amplification product, or wherein said label is a molecule or a chemical group which can be detected by a fluorescently labelled molecule, such as an antibody.

107. The method of any of claims 98 and 99, wherein the nucleotides of the 3′ overhang each comprises a nucleobase and a backbone unit, wherein the backbone unit comprises a sugar moiety and an internucleoside linker.

108. The method of claim 107, wherein the nucleobase of the nucleotides of the 3′ overhang are selected from naturally occurring nucleobases and non-naturally occurring nucleobases.

109. The method of claim 107, wherein the backbone unit of neighbouring nucleobases is selected from naturally occurring backbone units and non-naturally occurring backbone units.

110. The method of claim 107, wherein the sugar moiety of the backbone unit of neighbouring nucleobases is selected from naturally occurring sugar moieties and non-naturally occurring sugar moieties.

111. The method of claim 107, wherein the internucleoside linker of the backbone unit of neighbouring nucleobases is selected from naturally occurring internucleoside linkers and non-naturally occurring internucleoside linkers.

112. The method of claim 108, wherein the nucleobases of the 3′ overhang are selected independently from the group consisting of natural and non-natural purine heterocycles, natural and non-natural pyrimidine heterocycles, including heterocyclic, non-natural analogues and tautomers of said natural purine heterocycles and said natural pyrimidine heterocycles.

113. The method of claim 108, wherein the nucleobases of the 3′ overhang are selected independently from the group consisting of adenine, guanine, isoguanine, thymine, cytosine, isocytosine, pseudoisocytosine, uracil, inosine, purine, xanthine, diaminopurine, 8-oxo-N⁶-methyladenine, 7-deazaxanthine, 7-deazaguanine, N⁴,N⁴-ethanocytosin, N⁶,N⁶-ethano-2,6-diamino-purine, 5-methylcytosine, 5-(C³—C⁶)-alkynylcytosine, 5-fluorouracil, 5-bromouracil and 2-hydroxy-5-methyl-4-triazolopyridine.

114. The method of claim 108, the nucleobases of the 3′ overhang are selected independently from the group consisting of adenine, guanine, thymine, cytosine, 5-methylcytosine and uracil.

115. The method of claim 109, wherein the backbone units of the nucleotides of the 3′ overhang are the same or different backbone units.

116. The method of claim 115, wherein the same or different backbone units of the nucleotides of the 3′ overhang are selected independently from the group consisting of 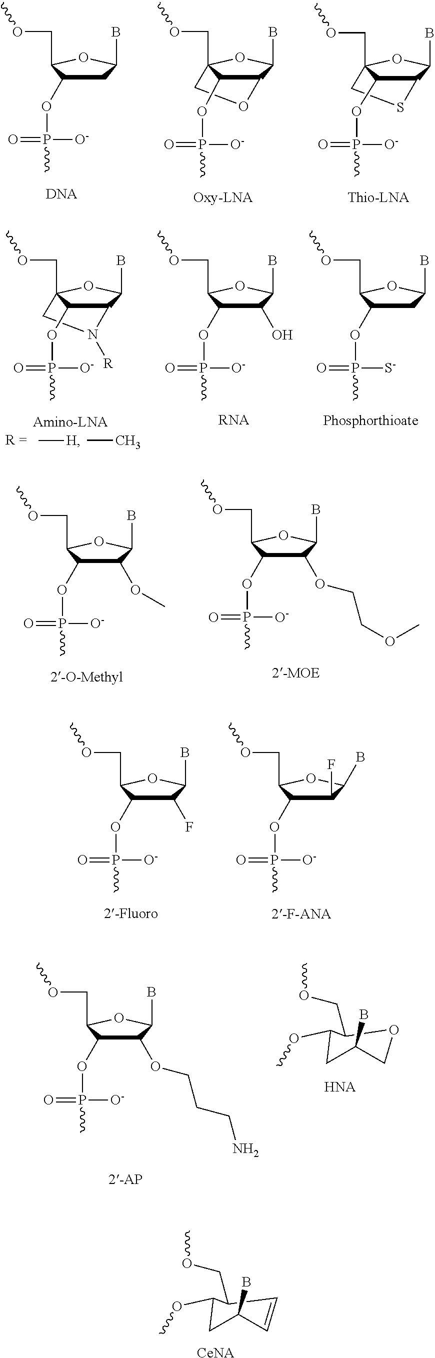 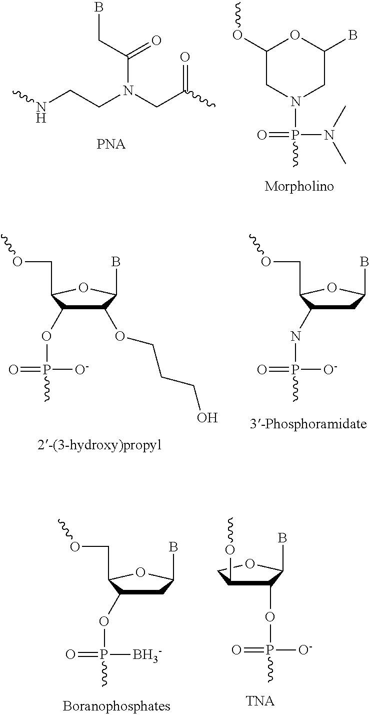
wherein B denotes a nucleobase.

117. The method of claim 110, wherein the sugar moiety of the backbone unit of the nucleotides of the 3′ overhang comprises or consists of a pentose.

118. The method of claim 117, wherein the pentose is selected from the group consisting of ribose, 2′-deoxyribose, 2′-O-methyl-ribose, 2′-fluor-ribose, and 2′-4′-O-methylene-ribose (LNA).

119. The method of any of claims 117 and 118, wherein the nucleobase of the nucleotide is attached to the 1′ position of the pentose.

120. The method of claim 119, wherein the backbone units linking any two neighbouring nucleotides of the 3′ overhang are the same or different backbone units.

121. The method of claim 120, wherein at least some of the nucleotides of the 3′ overhang are linked by different backbone units.

122. The method of claim 121, wherein at least some of said different backbone units are non-natural backbone units.

123. The method of claim 121, wherein the internucleoside linkers linking any two neighbouring nucleotides of the 3′ overhang are the same or different internucleoside linkers.

124. The method of claim 121, wherein at least some of the nucleotides of the 3′ overhang are linked by different internucleoside linkers.

125. The method of claim 124, wherein at least some of said different internucleotide linkers are non-natural internucleotide linkers.

126. The method of claim 121, wherein the internucleoside linkers of the 3′ overhang are selected from the group consisting of phosphodiester bonds, phosphorothioate bonds, methylphosphonate bonds, phosphoramidate bonds, phosphotriester bonds and phosphodithioate bonds.

127. The method of claim 121, wherein the internucleoside linkers of the 3′ overhang are selected from the group consisting of phosphorothioate bonds, methylphosphonate bonds, phosphoramidate bonds, phosphotriester bonds and phosphodithioate bonds.

128. The method of any of claims 98 and 99, wherein the nucleotides of the 3′ overhang are selected from naturally occurring nucleosides of the DNA and RNA family connected through phosphodiester linkages and at least one non-natural nucleotide selected from the group consisting of nucleotides comprising a non-natural nucleobase and/or a non-natural backbone unit comprising a non-natural sugar moiety and/or a non-natural internucleoside linker.

129. The method of claim 128, wherein the naturally occurring nucleosides are deoxynucleosides selected from the group consisting of deoxyadenosine, deoxyguanosine, deoxythymidine, and deoxycytidine.

130. The method of claim 128, wherein the naturally occurring nucleosides are selected from the group of nucleotides consisting of adenosine, guanosine, uridine, cytidine, and inosine.

131. The method of any of claims 128 to 130, wherein the non-natural nucleobase of the one or more non-natural nucleotides is selected from the group consisting of 8-oxo-N⁶-methyladenine, 7-deazaxanthine, 7-deazaguanine, N⁴,N⁴-ethanocytosin, N⁶,N⁶-ethano-2,6-diamino-purine, 5-methylcytosine, 5-(C³—C⁶)-alkynylcytosine, 5-fluorouracil, 5-bromouracil, pseudoisocytosine, 2-hydroxy-5-methyl-4-triazolopyridine, isocytosine, isoguanine and inosine.

132. The method of any of claims 128 to 131, wherein the non-natural backbone unit of the one or more non-natural nucleotides is selected from the group consisting of 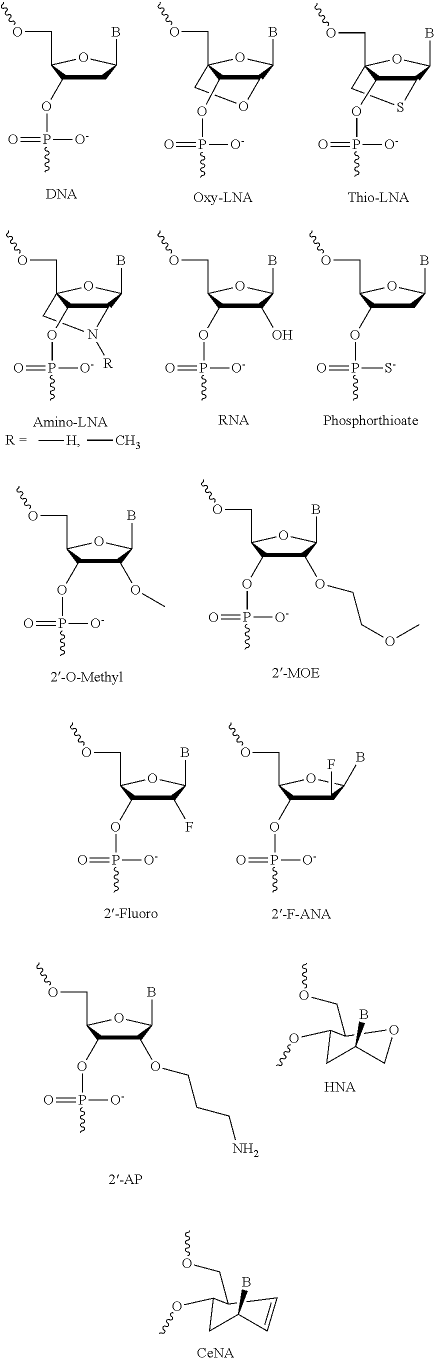 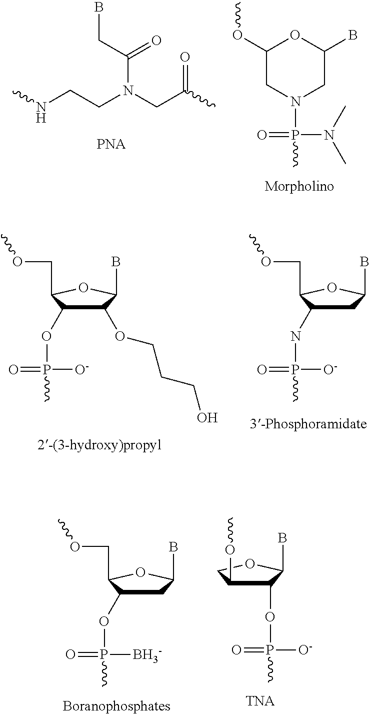
wherein B denotes a nucleobase.

133. The method of any of claims 128 to 132, wherein the non-natural sugar moiety of the one or more non-natural backbone unit(s) is selected from the group consisting of 2′-deoxyribose, 2′-O-methyl-ribose, 2′-fluor-ribose and 2′-4′-O-methylene-ribose (LNA).

134. The method of any of claims 128 to 133, wherein the non-natural internucleoside linker of the one or more non-natural backbone unit(s) is selected from the group consisting of phosphorothioate bonds, methylphosphonate bonds, phosphoramidate bonds, phosphotriester bonds and phosphodithioate bonds.

135. The method of claim 108, wherein said 3′ overhang comprises naturally occurring nucleobases connected by naturally occurring backbone units, wherein said naturally occurring nucleobases and said naturally occurring backbone units do not prevent exonuclease degradation of said 3′ overhang.

136. The method of claim 135, wherein said 3′ overhang further comprises non-naturally occurring nucleobases which do not prevent exonuclease degradation of said 3′ overhang.

137. The method of claim 136, wherein said non-naturally occurring backbone units comprising sugar moieties and internucleoside linkers do not prevent exonuclease degradation of said 3′ overhang.

138. The method of claim 137, wherein said sugar moieties are non-naturally occurring sugar moieties which do not prevent exonuclease degradation of said 3′ overhang.

139. The method of claim 137, wherein said internucleoside linkers are non-naturally occurring internucleoside linkers which do not prevent exonuclease degradation of said 3′ overhang.

140. The method of any of claims 135 to 139, wherein said one or more enzyme activities present in said sample comprises a 3′ to 5′ exonuclease activity capable of cleaving one or more, such as all of the internucleoside linkers connecting the nucleotides of the 3′ overhang and/or a ligase activity.

141. The method of claim 140, wherein the circular oligonucleotide template capable of being amplified by rolling circle amplification is generated by ligating the oligonucleotide probe comprising a substrate moiety processed by 3′ to 5′ exonucleolytically digestion of the 3′ overhang, said ligation being performed by at least one ligase activity present in said sample.

142. The method of claim 141, wherein said circular, oligonucleotide template is amplified by rolling circle amplification, said amplification being indicative of the presence in said sample of at least one 3′ to 5′ exonuclease activity and at least one ligase activity.

143. The method of claim 142, wherein said rolling circle amplification product is detected by detecting a label covalently or non-covalently associated with said rolling circle amplification product, wherein said label is preferably fluorescently detectable, wherein said label is either a fluorescent molecule incorporated into the rolling circle amplification product, for example by being present in the primer used for probe amplification and generation of the rolling circle amplification product, or by being linked to a nucleotide incorporated into the rolling circle amplification product during the probe amplification process, or by being linked to a fluorescently labelled oligonucleotide hybridising to the rolling circle amplification product, or wherein said label is a molecule or a chemical group which can be detected by a fluorescently labelled molecule, such as an antibody.

144. The method of claim 108, wherein said 3′ overhang comprises non-naturally occurring nucleobases connected by naturally occurring backbone units and/or non-naturally occurring backbone units, said backbone units comprising a sugar moiety and an internucleoside linker, wherein said non-naturally occurring nucleobases and said non-naturally occurring backbone units, when present, prevent exonuclease degradation of said 3′ overhang.

145. The method of claim 144, wherein said non-naturally occurring nucleobases alone prevents exonuclease degradation of said 3′ overhang.

146. The method of claim 144, wherein said non-naturally occurring backbone units prevent exonuclease degradation of said 3′ overhang.

147. The method of claim 144, wherein said non-naturally occurring sugar moieties prevent exonuclease degradation of said 3′ overhang.

148. The method of claim 144, wherein said non-naturally occurring internucleoside linkers prevent exonuclease degradation of said 3′ overhang.

149. The method of any of claims 144 to 148, wherein a 3′ to 5′ exonuclease activity present in said sample cannot cleave the internucleoside linkers connecting the nucleotides of the 3′ overhang.

150. The method of any of claims 144 to 148, wherein said one or more enzyme activities present in said sample further comprises a topoisomerase II activity capable of processing said unprocessed substrate moiety of said oligonucleotide probe.

151. The method of claim 150, wherein a circular, oligonucleotide template capable of being amplified by rolling circle amplification is generated by said toposiomerase II activity.

152. The method of claim 151, wherein said circular, oligonucleotide template is amplified by rolling circle amplification, said amplification being indicative of the presence in said sample of at least one topoisomerase II activity.

153. The method of claim 152, wherein said rolling circle amplification product is detected by detecting a label covalently or non-covalently associated with said rolling circle amplification product, wherein said label is preferably fluorescently detectable, wherein said label is either a fluorescent molecule incorporated into the rolling circle amplification product, for example by being present in the primer used for probe amplification and generation of the rolling circle amplification product, or by being linked to a nucleotide incorporated into the rolling circle amplification product during the probe amplification process, or by being linked to a fluorescently labelled oligonucleotide hybridising to the rolling circle amplification product, or wherein said label is a molecule or a chemical group which can be detected by a fluorescently labelled molecule, such as an antibody.

154. A liquid composition comprising
a) one or more oligonucleotide probes selected from the group consisting of
i) oligonucleotide probes comprising unprocessed substrate moieties comprising or consisting of one or more nick(s) in one or more single strand(s) of a double stranded nucleotide sequence of said oligonucleotide probe, said one or more nick(s) forming one or more unprocessed substrate moieties of said oligonucleotide probe,
ii) oligonucleotide probes comprising unprocessed substrate moieties comprising or consisting of one or more single stranded nucleotide sequence(s) joined at one or both ends thereof by a double stranded nucleotide sequence, said single stranded sequence(s) creating one or more gap structure(s) forming one or more unprocessed substrate moieties of said oligonucleotide probe, and
iii) oligonucleotide probes comprising unprocessed substrate moieties comprising or consisting of one or more nick(s) or one or more gap(s), said gap(s) being in the form of a single stranded nucleotide sequence, said nick(s) or gap(s) being joined at one end thereof to a double stranded nucleotide sequence and at the other end thereof to at least one single stranded overhang joined to a double stranded nucleotide sequence of said oligonucleotide probe, wherein said nick(s) or gap(s) in combination with the at least one single stranded overhang forms one or more unprocessed substrate moieties of said oligonucleotide probe;
and
b) a liquid carrier, such as an aqueous solvent, allowing one or more enzymes to process the one or more unprocessed substrate moieties of said one or more oligonucleotide probes.

155. A composition comprising a tissue sample, or a biopsy sample, obtained from an animal, such as a human being, and the liquid composition according to claim 154.

156. A solid support comprising a plurality of attachment points for the attachment to the solid support of one or more oligonucleotide probes each comprising one or more unprocessed substrate moieties, wherein an oligonucleotide probe is either directly attached to an attachment point through one strand of the oligonucleotide probe, wherein said strand is capable of initiating rolling circle amplification of a second strand of the oligonucletide probe, or an oligonucleotide probe is attached to an attachment point through hybridisation of the oligonucleotide probe to a primer oligonucleotide attached to an attachment point, wherein said primer is capable of initiating rolling circle amplification of the oligonucletide probe, so that individual attachment points are associated with one or more oligonucleotide primers suitable for initiating rolling circle amplification of a circular template generated by enzyme processing of said one or more oligonucleotide probes each comprising one or more unprocessed substrate moieties,
wherein the same or different primers are associated with the same or different attachment points,
wherein the oligonucleotide probes attached to the solid support are selected from the group consisting of
i) oligonucleotide probes comprising unprocessed substrate moieties comprising or consisting of one or more nick(s) in one or more single strand(s) of a double stranded nucleotide sequence of said oligonucleotide probe, said one or more nick(s) forming one or more unprocessed substrate moieties of said oligonucleotide probe,
ii) oligonucleotide probes comprising unprocessed substrate moieties comprising or consisting of one or more single stranded nucleotide sequence(s) joined at one or both ends thereof by a double stranded nucleotide sequence, said single stranded sequence(s) creating one or more gap structure(s) forming one or more unprocessed substrate moieties of said oligonucleotide probe, and
iii) oligonucleotide probes comprising unprocessed substrate moieties comprising or consisting of one or more nick(s) or one or more gap(s), said gap(s) being in the form of a single stranded nucleotide sequence, said nick(s) or gap(s) being joined at one end thereof to a double stranded nucleotide sequence and at the other end thereof to at least one single stranded overhang joined to a double stranded nucleotide sequence of said oligonucleotide probe, wherein said nick(s) or gap(s) in combination with the at least one single stranded overhang forms one or more unprocessed substrate moieties of said oligonucleotide probe.

157. The solid support according to claim 156, wherein each oligonucleotide probe attached to the attachment site at a different, predetermined position comprises the same or a different nucleotide or sequence of nucleotides for use in probe detection and/or probe confirmation.

158. The solid support according to claim 156, wherein said primer is associated with one or more label(s) selected from the group consisting of chromophores and fluorophores.

159. The solid support according to any of claims 156 to 158, wherein some or all of said oligonucleotide probes further comprise one or more non-hybridised, single stranded portion(s) and one or more double stranded portion(s), each double stranded portion comprising complementary nucleotide strands.

160. The solid support according to claim 159, wherein said one or more single stranded portion(s) of said oligonucleotide probes does not hybridise to a complementary nucleotide sequence.

161. The solid support according to claim 159, wherein said some or all of said oligonucleotide probes comprise at least one nucleotide sequence which is complementary to one or more of said single stranded portion(s) of the same oligonucleotide probe.

162. The solid support according to any of claims 156 to 161, wherein some or all of said oligonucleotide probes are in the form of a single oligonucleotide comprising a contiguous sequence of nucleotides, wherein at least some of said nucleotides are capable of forming a double stranded sequence comprising complementary nucleotide strands.

163. The solid support according to any of claims 156 to 161, wherein some or all of said oligonucleotide probes comprise more than one single oligonucleotide, wherein each oligonucleotide of each probe comprises a single contiguous sequence of nucleotides, wherein at least some of said nucleotides of the different oligonucleotides of the probe are capable of hybridising to each other, and wherein, preferably, at least one of said more than one single oligonucleotides are capable of priming rolling circle amplification of another oligonucleotide.

164. The solid support according to any of claims 156 to 163, wherein some or all of the probes are self-templating probes each comprising at least two double stranded portions each comprising complementary nucleotide strands separated at the proximal ends thereof by an unprocessed substrate moiety.

165. The solid support according to claim 164, wherein the at least two double stranded portions comprising complementary nucleotide strands are each joined at the distal ends thereof by a single stranded nucleotide forming a loop structure.

166. The solid support according to any of claims 156 to 165, wherein some or all of the oligonucleotides comprise one or more unprocessed substrate moieties each comprising a nick or a single stranded nucleotide region.

167. The solid support according to any of claims 156 to 166, wherein the substrate moiety conversion of at least some of the oligonucleotide probes is capable of being mediated specifically by an enzyme capable of performing template directed nucleotide synthesis and/or a ligase.

168. The solid support according to claim 166, wherein the single stranded nucleotide region is adjoined at either end thereof to a double stranded nucleotide region.

169. The solid support according to any of claims 167 and 168, wherein the single stranded nucleotide region is a 5′ overhang nucleotide region adjoined at one end thereof to a double stranded nucleotide region of the oligonucleotide probe.

170. The solid support according to any of claims 167 and 168, wherein the single stranded nucleotide region is a 3′ overhang nucleotide region adjoined at one end thereof to a double stranded nucleotide region of the oligonucleotide probe.

171. The solid support according to any of claims 166 to 170, wherein the single stranded nucleotide region preferably contains less than 20 nucleotides.

172. The solid support according to any of claims 166 to 170, wherein the single stranded nucleotide region preferably contains less than 15 nucleotides.

173. The solid support according to any of claims 166 to 170, wherein the single stranded nucleotide region preferably contains less than 10 nucleotides.

174. The solid support according to any of claims 166 to 170, wherein the single stranded nucleotide region preferably contains less than 5 nucleotides.

175. The solid support according to any of claims 166 to 170, wherein the single stranded nucleotide region preferably contains less than 3 nucleotides.

176. The solid support according to any of claims 166 to 174, wherein the substrate moiety conversion of at least some of the oligonucleotide probes is capable of being mediated specifically by a flap endonuclease activity in combination with a ligase activity.

177. The solid support according to claim 176, wherein the flap endonuclease activity is mediated by one or more of FEN1, DNA2P and EXO1.

178. The solid support according to any of claims 166 to 174, wherein the substrate moiety conversion of at least some of the oligonucleotide probes is capable of being mediated specifically by a topoisomerase activity.

179. The solid support according to claim 178, wherein the topoisomerase activity is mediated by a Topoisomerase I.

180. The solid support according to claim 178, wherein the topoisomerase activity is mediated by a Topoisomerase II.

181. The solid support according to any of claims 156 to 174, wherein at least 3 different types of oligonucleotide probes are associated with said solid support through hybridisation to one or more oligonucleotide primers each associated with a solid support attachment point, wherein each of said 3 different types of oligonucleotide probes comprises an unprocessed substrate moiety, wherein the unprocessed substrate moiety of each type of oligonucleotide probe is different and each type of oligonucleotide probe is capable of being processed by at least one different enzyme, wherein said at least one different enzyme is selected from the group consisting of a ligase, an exonuclease, such as a 5′ to 3′ exonuclease or a 3′ to 5′ exonuclease, and an endonuclease, such as a flap endonuclease, such as FEN1 or DNA2P and EXO1, or a topoisomerase, such as a topoisomerase of type I or type II.

182. The solid support according to claim 181, wherein said different 3 types of oligonucleotide probes are
i) oligonucleotide probes comprising unprocessed substrate moieties comprising or consisting of one or more nick(s) in one or more single strand(s) of a double stranded nucleotide sequence of said oligonucleotide probe, said one or more nick(s) forming one or more unprocessed substrate moieties of said oligonucleotide probe, and
ii) oligonucleotide probes comprising unprocessed substrate moieties comprising or consisting of one or more single stranded nucleotide sequence(s) joined at one or both ends thereof by a double stranded nucleotide sequence, said single stranded sequence(s) creating one or more gap structure(s) forming one or more unprocessed substrate moieties of said oligonucleotide probe, and
iii) oligonucleotide probes comprising unprocessed substrate moieties comprising or consisting of one or more nick(s) or one or more gap(s), said gap(s) being in the form of a single stranded nucleotide sequence, said nick(s) or gap(s) being joined at one end thereof to a double stranded nucleotide sequence and at the other end thereof to at least one single stranded overhang joined to a double stranded nucleotide sequence of said oligonucleotide probe, wherein said nick(s) or gap(s) in combination with the at least one single stranded overhang forms one or more unprocessed substrate moieties of said oligonucleotide probe.

183. The solid support according to any of claims 156 and 182, wherein said solid support is associated with oligonucleotide probes each comprising one or more unprocessed substrate moieties each comprising or consisting of one or more nick(s) in one or more single strand(s) of a double stranded nucleotide sequence of some or all of said oligonucleotide probes, said one or more nick(s) forming one or more unprocessed substrate moieties of said oligonucleotide probe.

184. The solid support according to claim 183, wherein said oligonucleotide probes comprising said one or more unprocessed substrate moieties are capable of being converted to a circular oligonucleotide template by a ligase activity capable of ligating said one or more nick(s) of said oligonucleotide probe.

185. The solid support according to any of claims 183 and 184, wherein said circular oligonucleotide templates generated by ligation of said nick(s) are amplified in situ by rolling circle amplification initiated by a polymerase and the primer associated with a predetermined attachment point of the solid support, said rolling circle amplification generating a rolling circle amplification product which remains associated with an attachment point of said solid support.

186. The solid support according to claim 185, wherein said solid support further comprises detection means for detection of said rolling circle amplification product.

187. The solid support according to claim 186, wherein said rolling circle amplification product is detected by detecting a label covalently or non-covalently associated with said rolling circle amplification product, wherein said label is preferably fluorescently detectable, wherein said label is either a fluorescent molecule incorporated into the rolling circle amplification product, for example by being present in the primer used for probe amplification and generation of the rolling circle amplification product, or by being linked to a nucleotide incorporated into the rolling circle amplification product during the probe amplification process, or by being linked to a fluorescently labelled oligonucleotide hybridising to the rolling circle amplification product, or wherein said label is a molecule or a chemical group which can be detected by a fluorescently labelled molecule, such as an antibody.

188. The solid support according to any of claims 156 and 182, wherein said solid support is associated with oligonucleotide probes each comprising one or more unprocessed substrate moieties comprising or consisting of one or more single stranded nucleotide sequence(s) joined at one or both ends thereof by a double stranded nucleotide sequence, said single stranded sequence(s) creating one or more gap structure(s) forming said one or more unprocessed substrate moieties of each of said oligonucleotide probe(s).

189. The solid support according to claim 188, wherein said oligonucleotide probes comprising said one or more unprocessed substrate moieties are capable of being converted to a circular oligonucleotide template by a) filling-in said gap by using the at least one enzyme capable of performing a template directed nucleotide extension reaction, and b) ligating the end-positioned, filled-in nucleotides to the remaining, double stranded part of the oligonucleotide probe.

190. The solid support according to any of claims 188 and 189, wherein said circular oligonucleotide templates generated by filling-in and ligating said gap structure is amplified in situ by rolling circle amplification initiated by a polymerase and the primer associated with a predetermined attachment point of the solid support, said rolling circle amplification generating a rolling circle amplification product which remains associated with an attachment point of said solid support.

191. The solid support according to claim 190, wherein said solid support further comprises detection means for detection of said rolling circle amplification product.

192. The solid support according to claim 191, wherein said rolling circle amplification product can be detected by detecting a label covalently or non-covalently associated with said rolling circle amplification product, wherein said label is preferably fluorescently detectable, wherein said label is either a fluorescent molecule incorporated into the rolling circle amplification product, for example by being present in the primer used for probe amplification and generation of the rolling circle amplification product, or by being linked to a nucleotide incorporated into the rolling circle amplification product during the probe amplification process, or by being linked to a fluorescently labelled oligonucleotide hybridising to the rolling circle amplification product, or wherein said label is a molecule or a chemical group which can be detected by a fluorescently labelled molecule, such as an antibody.

193. The solid support according to any of claims 156 and 182, wherein said solid support is associated with oligonucleotide probes each comprising one or more unprocessed substrate moieties comprising or consisting of one or more nick(s) and/or one or more gap(s), said gap(s) being in the form of a single stranded nucleotide sequence, said nick(s) or gap(s) being joined at one end thereof to a double stranded nucleotide sequence and at the other end thereof to at least one single stranded overhang joined to a double stranded nucleotide sequence of said oligonucleotide probe, wherein said nick(s) or gap(s) in combination with the at least one single stranded overhang forms said one or more unprocessed substrate moieties of said oligonucleotide probe.

194. The solid support according to claim 193, wherein the one or more overhang(s) is a 5′ overhang, said oligonucleotide probe further comprising at least one 3′ end.

195. The solid support according to claim 194, wherein the 5′ overhang is protected by a protection group capable of preventing an exonuclease from digesting the 5′ overhang.

196. The solid support according to claim 195, wherein said oligonucleotide probes comprising said one or more unprocessed substrate moieties are capable of being converted to a circular oligonucleotide template by a) endonucleolytic digestion of said 5′ overhang and b) ligation of the end of the nucleotide strand resulting from the endonucleolytic digestion to a nucleotide strand of the remaining part of the oligonucleotide probe, thereby generating a circular oligonucleotide template.

197. The solid support according to claim 196, wherein said circular oligonucleotide templates generated by endonucleolytic cleavage and ligation is amplified in situ by rolling circle amplification initiated by a polymerase and the primer associated with a predetermined attachment point of the solid support, said rolling circle amplification generating a rolling circle amplification product which remains associated with an attachment point of said solid support.

198. The solid support according to claim 197, wherein said solid support further comprises detection means for detection of said rolling circle amplification product.

199. The solid support according to claim 198, wherein said rolling circle amplification product is detected by detecting a label covalently or non-covalently associated with said rolling circle amplification product, wherein said label is preferably fluorescently detectable, wherein said label is either a fluorescent molecule incorporated into the rolling circle amplification product, for example by being present in the primer used for probe amplification and generation of the rolling circle amplification product, or by being linked to a nucleotide incorporated into the rolling circle amplification product during the probe amplification process, or by being linked to a fluorescently labelled oligonucleotide hybridising to the rolling circle amplification product, or wherein said label is a molecule or a chemical group which can be detected by a fluorescently labelled molecule, such as an antibody.

200. The solid support according to any of claims 194 and 195, wherein the 5′ end of the 5′ overhang of the oligonucleotide probes comprises a protection group different from a phosphate group, wherein said protection group prevents ligation of said 5′ overhang to a 3′ end of a strand of the remaining part of the oligonucleotide probe.

201. The solid support according to claim 200, wherein said protection group different from a phosphate group is selected from the group consisting of H, biotin, amin, and an optionally substituted C₁-C₆-linker.

202. The solid support according to any of claims 200 and 201, wherein the protection group different from a phosphate group of the unprocessed substrate moiety of said oligonucleotide probe prevents said oligonucleotide from being processed and circularised by a topoisomerase I activity.

203. The solid support according to claim 202, wherein the unprocessed substrate moiety is processed by a flap endonuclease, wherein said processing results in the formation of a 5′ end having a phosphate reactive group capable of being ligated with a 3′ end of a strand of the remaining part of the oligonucleotide probe, thereby generating a circular oligonucleotide template capable of being amplified in situ by rolling circle amplification initiated by a polymerase and the primer associated with a predetermined attachment point of the solid support, said rolling circle amplification generating a rolling circle amplification product which remains associated with an attachment point of said solid support.

204. The solid support according to claim 203, wherein said solid support further comprises one or more rolling circle amplification products generated by in situ amplification of said circular, oligonucleotide template generated by the combined action of said flap endonuclease and a ligase.

205. The solid support according to claim 204, wherein said solid support further comprises means for detection of said rolling circle amplification product.

206. The solid support according to any of claims 194 and 195, wherein said 3′ end of the oligonucleotide probe comprises a protection group different from a hydroxy group, wherein said protection group prevents ligation of said 5′ overhang to the 3′ end of a strand of the remaining part of the oligonucleotide probe.

207. The solid support according to claim 206, wherein said protection group different from a hydroxy group is selected from the group consisting of H, biotin, amin, and an optionally substituted C₁-C₆-linker.

208. The solid support according to any of claims 206 and 207, wherein the protection group different from a hydroxy group of the unprocessed substrate moiety of said oligonucleotide probe prevents said oligonucleotide from being processed and circularised by a flap endonuclease activity in combination with a ligase.

209. The solid support according to claim 208, wherein a topoisomerase I activity present in a biological sample processes the unprocessed substrate moiety of said oligonucleotide probe, said processing resulting in the formation of a 3′-phospho-tyrosine intermediate (covalent DNA-protein intermediate) capable of being ligated with the HO-group of the 5′-end of the 5′-overhang of the oligonucleotide probe, wherein said ligation results in the formation of a circular oligonucleotide template capable of being amplified by rolling circle amplification initiated by a polymerase and the primer associated with a predetermined attachment point of the solid support, said rolling circle amplification generating a rolling circle amplification product which remains associated with an attachment point of said solid support.

210. The solid support according to claim 209, wherein said solid support further comprises means for detection of said rolling circle amplification product.

211. The solid support according to claim 210, wherein said rolling circle amplification product is detected by detecting a label covalently or non-covalently associated with said rolling circle amplification product, wherein said label is preferably fluorescently detectable, wherein said label is either a fluorescent molecule incorporated into the rolling circle amplification product, for example by being present in the primer used for probe amplification and generation of the rolling circle amplification product, or by being linked to a nucleotide incorporated into the rolling circle amplification product during the probe amplification process, or by being linked to a fluorescently labelled oligonucleotide hybridising to the rolling circle amplification product, or wherein said label is a molecule or a chemical group which can be detected by a fluorescently labelled molecule, such as an antibody.

212. The solid support according to any of claims 156 and 182, wherein the nucleotides of the 5′ overhang each comprises a nucleobase and a backbone unit, wherein the backbone unit comprises a sugar moiety and an internucleoside linker.

213. The solid support according to claim 212, wherein the nucleobase of the nucleotides of the 5′ overhang are selected from naturally occurring nucleobases and non-naturally occurring nucleobases.

214. The solid support according to claim 212, wherein the backbone unit of neighbouring nucleobases is selected from naturally occurring backbone units and non-naturally occurring backbone units.

215. The solid support according to claim 212, wherein the sugar moiety of the backbone unit of neighbouring nucleobases is selected from naturally occurring sugar moieties and non-naturally occurring sugar moieties.

216. The solid support according to claim 212, wherein the internucleoside linker of the backbone unit of neighbouring nucleobases is selected from naturally occurring internucleoside linkers and non-naturally occurring internucleoside linkers.

217. The solid support according to claim 213, wherein the nucleobases of the 5′ overhang are selected independently from the group consisting of natural and non-natural purine heterocycles, natural and non-natural pyrimidine heterocycles, including heterocyclic, non-natural analogues and tautomers of said natural purine heterocycles and said natural pyrimidine heterocycles.

218. The solid support according to claim 213, wherein the nucleobases of the 5′ overhang are selected independently from the group consisting of adenine, guanine, isoguanine, thymine, cytosine, isocytosine, pseudoisocytosine, uracil, inosine, purine, xanthine, diaminopurine, 8-oxo-N⁶-methyladenine, 7-deazaxanthine, 7-deazaguanine, N⁴,N⁴-ethanocytosin, N⁶,N⁶-ethano-2,6-diamino-purine, 5-methylcytosine, 5-(C³—C⁶)-alkynylcytosine, 5-fluorouracil, 5-bromouracil and 2-hydroxy-5-methyl-4-triazolopyridine.

219. The solid support according to claim 213, the nucleobases of the 5′ overhang are selected independently from the group consisting of adenine, guanine, thymine, cytosine, 5-methylcytosine and uracil.

220. The solid support according to claim 214, wherein the backbone units of the nucleotides of the 5′ overhang are the same or different backbone units.

221. The solid support according to claim 220, wherein the same or different backbone units of the nucleotides of the 5′ overhang are selected independently from the group consisting of 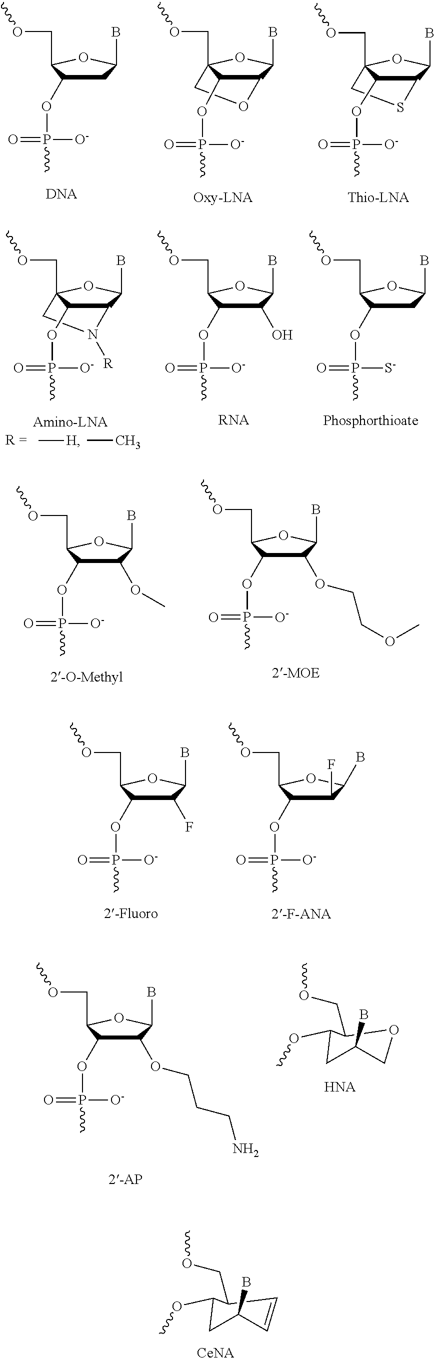 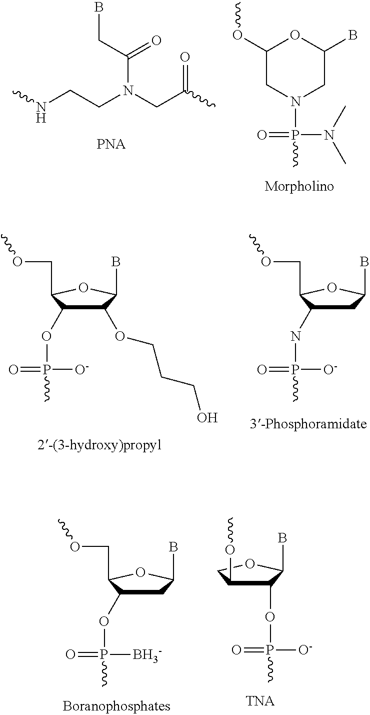
wherein B denotes a nucleobase.

222. The solid support according to claim 215, wherein the sugar moiety of the backbone unit of the nucleotides of the 5′ overhang comprises or consists of a pentose.

223. The solid support according to claim 222, wherein the pentose is selected from the group consisting of ribose, 2′-deoxyribose, 2′-O-methyl-ribose, 2′-fluor-ribose, and 2′-4′-O-methylene-ribose (LNA).

224. The solid support according to any of claims 222 and 223, wherein the nucleobase of the nucleotide is attached to the 1′ position of the pentose.

225. The solid support according to claim 224, wherein the backbone units linking any two neighbouring nucleotides of the 5′ overhang are the same or different backbone units.

226. The solid support according to claim 225, wherein at least some of the nucleotides of the 5′ overhang are linked by different backbone units.

227. The solid support according to claim 226, wherein at least some of said different backbone units are non-natural backbone units.

228. The solid support according to claim 216, wherein the internucleoside linkers linking any two neighbouring nucleotides of the 5′ overhang are the same or different internucleoside linkers.

229. The solid support according to claim 216, wherein at least some of the nucleotides of the 5′ overhang are linked by different internucleoside linkers.

230. The solid support according to claim 229, wherein at least some of said different internucleotide linkers are non-natural internucleotide linkers.

231. The solid support according to claim 216, wherein the internucleoside linkers of the 5′ overhang are selected from the group consisting of phosphodiester bonds, phosphorothioate bonds, methylphosphonate bonds, phosphoramidate bonds, phosphotriester bonds and phosphodithioate bonds.

232. The solid support according to claim 216, wherein the internucleoside linkers of the 5′ overhang are selected from the group consisting of phosphorothioate bonds, methylphosphonate bonds, phosphoramidate bonds, phosphotriester bonds and phosphodithioate bonds.

233. The solid support according to any of claims 194 and 195, wherein the nucleotides of the 5′ overhang are selected from naturally occurring nucleosides of the DNA and RNA family connected through phosphodiester linkages and at least one non-natural nucleotide selected from the group consisting of nucleotides comprising a non-natural nucleobase and/or a non-natural backbone unit comprising a non-natural sugar moiety and/or a non-natural internucleoside linker.

234. The solid support according to claim 233, wherein the naturally occurring nucleosides are deoxynucleosides selected from the group consisting of deoxyadenosine, deoxyguanosine, deoxythymidine, and deoxycytidine.

235. The solid support according to claim 233, wherein the naturally occurring nucleosides are selected from the group of nucleotides consisting of adenosine, guanosine, uridine, cytidine, and inosine.

236. The solid support according to any of claims 233 to 235, wherein the non-natural nucleobase of the one or more non-natural nucleotides is selected from the group consisting of 8-oxo-N⁶-methyladenine, 7-deazaxanthine, 7-deazaguanine, N⁴,N⁴-ethanocytosin, N⁶,N⁶-ethano-2,6-diamino-purine, 5-methylcytosine, 5-(C³—C⁶)-alkynylcytosine, 5-fluorouracil, 5-bromouracil, pseudoisocytosine, 2-hydroxy-5-methyl-4-triazolopyridine, isocytosine, isoguanine and inosine.

237. The solid support according to any of claims 233 to 236, wherein the non-natural backbone unit of the one or more non-natural nucleotides is selected from the group consisting of 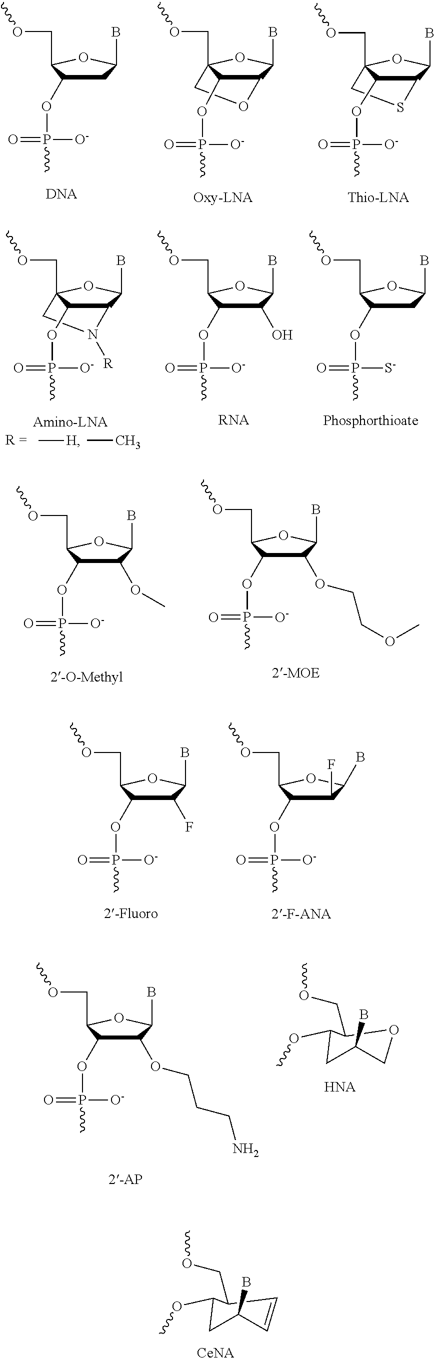 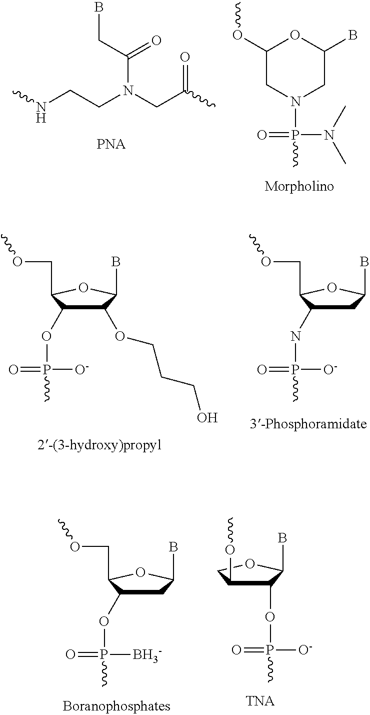
wherein B denotes a nucleobase.

238. The solid support according to any of claims 233 to 237, wherein the non-natural sugar moiety of the one or more non-natural backbone unit(s) is selected from the group consisting of 2′-deoxyribose, 2′-O-methyl-ribose, 2′-fluor-ribose and 2′-4′-O-methylene-ribose (LNA).

239. The solid support according to any of claims 233 to 237, wherein the non-natural internucleoside linker of the one or more non-natural backbone unit(s) is selected from the group consisting of phosphorothioate bonds, methylphosphonate bonds, phosphoramidate bonds, phosphotriester bonds and phosphodithioate bonds.

240. The solid support according to claim 213, wherein said 5′ overhang comprises naturally occurring nucleobases connected by naturally occurring backbone units, wherein said naturally occurring nucleobases and said naturally occurring backbone units do not prevent exonuclease degradation of said 5′ overhang.

241. The solid support according to claim 240, wherein said 5′ overhang further comprises non-naturally occurring nucleobases which do not prevent exonuclease degradation of said 5′ overhang.

242. The solid support according to claim 241, wherein said non-naturally occurring backbone units comprising sugar moieties and internucleoside linkers do not prevent exonuclease degradation of said 5′ overhang.

243. The solid support according to claim 242, wherein said sugar moieties are non-naturally occurring sugar moieties which do not prevent exonuclease degradation of said 5′ overhang.

244. The solid support according to claim 242, wherein said internucleoside linkers are non-naturally occurring internucleoside linkers which do not prevent exonuclease degradation of said 5′ overhang.

245. The solid support according to any of claims 240 to 244, wherein said one or more enzyme activities present in said sample comprises a 5′ to 3′ exonuclease activity capable of cleaving one or more, such as all of the internucleoside linkers connecting the nucleotides of the 5′ overhang and/or a ligase activity.

246. The solid support according to claim 245, wherein the circular oligonucleotide template capable of being amplified by rolling circle amplification is generated by ligating the oligonucleotide probe comprising a substrate moiety processed by 5′ to 3′ exonucleolytic digestion of the 5′ overhang, said ligation being performed by at least one ligase activity present in said sample.

247. The solid support according to claim 246, wherein said circular, oligonucleotide template is amplified by rolling circle amplification, said amplification being indicative of the presence in said sample of at least one 5′ to 3′ exonuclease activity and at least one ligase activity.

248. The solid support according to claim 246, wherein said rolling circle amplification product is detected by detecting a label covalently or non-covalently associated with said rolling circle amplification product, wherein said label is preferably fluorescently detectable, wherein said label is either a fluorescent molecule incorporated into the rolling circle amplification product, for example by being present in the primer used for probe amplification and generation of the rolling circle amplification product, or by being linked to a nucleotide incorporated into the rolling circle amplification product during the probe amplification process, or by being linked to a fluorescently labelled oligonucleotide hybridising to the rolling circle amplification product, or wherein said label is a molecule or a chemical group which can be detected by a fluorescently labelled molecule, such as an antibody.

249. The solid support according to claim 213, wherein said 5′ overhang comprises non-naturally occurring nucleobases connected by naturally occurring backbone units and/or non-naturally occurring backbone units, said backbone units comprising a sugar moiety and an internucleoside linker, wherein said non-naturally occurring nucleobases and said non-naturally occurring backbone units, when present, prevent exonuclease degradation of said 5′ overhang.

250. The solid support according to claim 249, wherein said non-naturally occurring nucleobases alone prevents exonuclease degradation of said 5′ overhang.

251. The solid support according to claim 249, wherein said non-naturally occurring backbone units prevent exonuclease degradation of said 5′ overhang.

252. The solid support according to claim 249, wherein said non-naturally occurring sugar moieties prevent exonuclease degradation of said 5′ overhang.

253. The solid support according to claim 249, wherein said non-naturally occurring internucleoside linkers prevent exonuclease degradation of said 5′ overhang.

254. The solid support according to any of claims 249 to 253, wherein a 5′ to 3′ exonuclease activity present in said sample cannot cleave the internucleoside linkers connecting the nucleotides of the 5′ overhang.

255. The solid support according to any of claims 249 to 253, wherein said one or more enzyme activities present in said sample further comprises a flap endonuclease activity capable of cleaving the internucleoside linkers connecting the nucleotides of the 5′ overhang and/or a ligase activity.

256. The solid support according to claim 255, wherein a circular, oligonucleotide template capable of being amplified by rolling circle amplification is generated by ligating the oligonucleotide probe comprising a processed substrate moiety, wherein said substrate moiety processing comprises flap endonucleolytically cleaving at least one internucleoside linker of the 5′ overhang of the probe, thereby releasing the 5′ overhang from the remaining part of the oligonucleotide probe, wherein the ligation is performed by at least one ligase activity present in said sample.

257. The solid support according to claim 256, wherein said circular, oligonucleotide template is amplified by rolling circle amplification, said amplification being indicative of the presence in said sample of at least one flap endonuclease activity and at least one ligase activity.

258. The solid support according to claim 257, wherein said rolling circle amplification product is detected by detecting a label covalently or non-covalently associated with said rolling circle amplification product, wherein said label is preferably fluorescently detectable, wherein said label is either a fluorescent molecule incorporated into the rolling circle amplification product, for example by being present in the primer used for probe amplification and generation of the rolling circle amplification product, or by being linked to a nucleotide incorporated into the rolling circle amplification product during the probe amplification process, or by being linked to a fluorescently labelled oligonucleotide hybridising to the rolling circle amplification product, or wherein said label is a molecule or a chemical group which can be detected by a fluorescently labelled molecule, such as an antibody.

259. The solid support according to claim 194, wherein the one or more overhang(s) is a 3′ overhang, said oligonucleotide probe further comprising at least one 5′ end.

260. The solid support according to claim 259, wherein the 3′ overhang is protected by a protection group preventing an exonuclease from digesting the 3′ overhang.

261. The solid support according to claim 260, wherein a circular, oligonucleotide template capable of being amplified by rolling circle amplification is generated by a) endonucleolytic digestion of said 3′ overhang and b) ligation of the end of the nucleotide strand resulting from the endonucleolytic digestion to a nucleotide strand of the remaining part of the oligonucleotide probe.

262. The solid support according to claim 261, wherein said circular, oligonucleotide template is amplified by rolling circle amplification, said amplification being indicative of the presence in said sample of at least one endonuclease.

263. The solid support according to claim 262, wherein said rolling circle amplification product is detected by detecting a label covalently or non-covalently associated with said rolling circle amplification product, wherein said label is preferably fluorescently detectable, wherein said label is either a fluorescent molecule incorporated into the rolling circle amplification product, for example by being present in the primer used for probe amplification and generation of the rolling circle amplification product, or by being linked to a nucleotide incorporated into the rolling circle amplification product during the probe amplification process, or by being linked to a fluorescently labelled oligonucleotide hybridising to the rolling circle amplification product, or wherein said label is a molecule or a chemical group which can be detected by a fluorescently labelled molecule, such as an antibody.

264. The solid support according to any of claims 259 and 260, wherein a topoisomerase II activity present in said sample processes the unprocessed substrate moiety of said oligonucleotide probe and thereby provides a circular oligonucleotide template.

265. The solid support according to claim 264, wherein said circular oligonucleotide template generated by the topoisomerase II activity present in said sample is amplified by rolling circle amplification, thereby generating a rolling circle amplification product.

266. The solid support according to claim 265, wherein said rolling circle amplification product is indicative of the presence in said sample of a topoisomerase II activity.

267. The solid support according to claim 266, wherein said rolling circle amplification product is detected by detecting a label covalently or non-covalently associated with said rolling circle amplification product, wherein said label is preferably fluorescently detectable, wherein said label is either a fluorescent molecule incorporated into the rolling circle amplification product, for example by being present in the primer used for probe amplification and generation of the rolling circle amplification product, or by being linked to a nucleotide incorporated into the rolling circle amplification product during the probe amplification process, or by being linked to a fluorescently labelled oligonucleotide hybridising to the rolling circle amplification product, or wherein said label is a molecule or a chemical group which can be detected by a fluorescently labelled molecule, such as an antibody.

268. The solid support according to any of claims 259 and 260, wherein the nucleotides of the 3′ overhang each comprises a nucleobase and a backbone unit, wherein the backbone unit comprises a sugar moiety and an internucleoside linker.

269. The solid support according to claim 268, wherein the nucleobase of the nucleotides of the 3′ overhang are selected from naturally occurring nucleobases and non-naturally occurring nucleobases.

270. The solid support according to claim 268, wherein the backbone unit of neighbouring nucleobases is selected from naturally occurring backbone units and non-naturally occurring backbone units.

271. The solid support according to claim 268, wherein the sugar moiety of the backbone unit of neighbouring nucleobases is selected from naturally occurring sugar moieties and non-naturally occurring sugar moieties.

272. The solid support according to claim 268, wherein the internucleoside linker of the backbone unit of neighbouring nucleobases is selected from naturally occurring internucleoside linkers and non-naturally occurring internucleoside linkers.

273. The solid support according to claim 269, wherein the nucleobases of the 3′ overhang are selected independently from the group consisting of natural and non-natural purine heterocycles, natural and non-natural pyrimidine heterocycles, including heterocyclic, non-natural analogues and tautomers of said natural purine heterocycles and said natural pyrimidine heterocycles.

274. The solid support according to claim 269, wherein the nucleobases of the 3′ overhang are selected independently from the group consisting of adenine, guanine, isoguanine, thymine, cytosine, isocytosine, pseudoisocytosine, uracil, inosine, purine, xanthine, diaminopurine, 8-oxo-N⁶-methyladenine, 7-deazaxanthine, 7-deazaguanine, N⁴,N⁴-ethanocytosin, N⁶,N⁶-ethano-2,6-diamino-purine, 5-methylcytosine, 5-(C³—C⁶)-alkynylcytosine, 5-fluorouracil, 5-bromouracil and 2-hydroxy-5-methyl-4-triazolopyridine.

275. The solid support according to claim 269, the nucleobases of the 3′ overhang are selected independently from the group consisting of adenine, guanine, thymine, cytosine, 5-methylcytosine and uracil.

276. The solid support according to claim 270, wherein the backbone units of the nucleotides of the 3′ overhang are the same or different backbone units.

277. The solid support according to claim 276, wherein the same or different backbone units of the nucleotides of the 3′ overhang are selected independently from the group consisting of 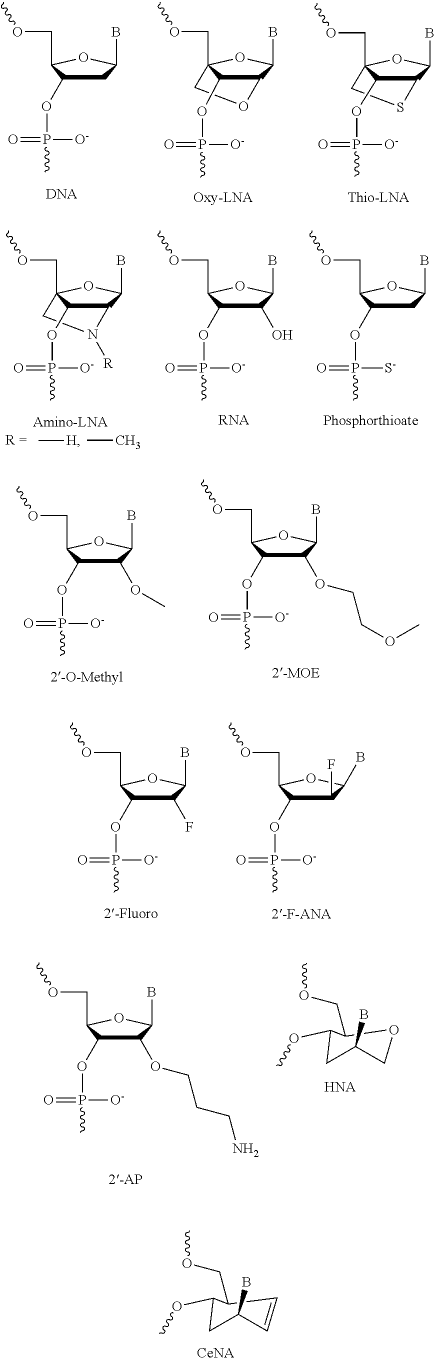 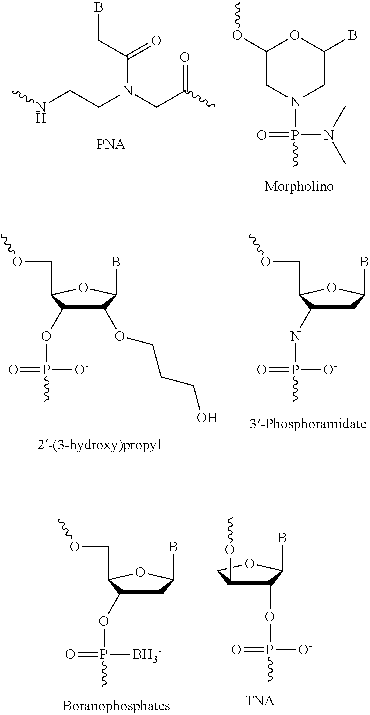
wherein B denotes a nucleobase.

278. The solid support according to claim 271, wherein the sugar moiety of the backbone unit of the nucleotides of the 3′ overhang comprises or consists of a pentose.

279. The solid support according to claim 278, wherein the pentose is selected from the group consisting of ribose, 2′-deoxyribose, 2′-O-methyl-ribose, 2′-fluor-ribose, and 2′-4′-O-methylene-ribose (LNA).

280. The solid support according to any of claims 278 and 279, wherein the nucleobase of the nucleotide is attached to the 1′ position of the pentose.

281. The solid support according to claim 280, wherein the backbone units linking any two neighbouring nucleotides of the 3′ overhang are the same or different backbone units.

282. The solid support according to claim 281, wherein at least some of the nucleotides of the 3′ overhang are linked by different backbone units.

283. The solid support according to claim 282, wherein at least some of said different backbone units are non-natural backbone units.

284. The solid support according to claim 282, wherein the internucleoside linkers linking any two neighbouring nucleotides of the 3′ overhang are the same or different internucleoside linkers.

285. The solid support according to claim 282, wherein at least some of the nucleotides of the 3′ overhang are linked by different internucleoside linkers.

286. The solid support according to claim 285, wherein at least some of said different internucleotide linkers are non-natural internucleotide linkers.

287. The solid support according to claim 282, wherein the internucleoside linkers of the 3′ overhang are selected from the group consisting of phosphodiester bonds, phosphorothioate bonds, methylphosphonate bonds, phosphoramidate bonds, phosphotriester bonds and phosphodithioate bonds.

288. The solid support according to claim 282, wherein the internucleoside linkers of the 3′ overhang are selected from the group consisting of phosphorothioate bonds, methylphosphonate bonds, phosphoramidate bonds, phosphotriester bonds and phosphodithioate bonds.

289. The solid support according to any of claims 259 and 260, wherein the nucleotides of the 3′ overhang are selected from naturally occurring nucleosides of the DNA and RNA family connected through phosphodiester linkages and at least one non-natural nucleotide selected from the group consisting of nucleotides comprising a non-natural nucleobase and/or a non-natural backbone unit comprising a non-natural sugar moiety and/or a non-natural internucleoside linker.

290. The solid support according to claim 289, wherein the naturally occurring nucleosides are deoxynucleosides selected from the group consisting of deoxyadenosine, deoxyguanosine, deoxythymidine, and deoxycytidine.

291. The solid support according to claim 289, wherein the naturally occurring nucleosides are selected from the group of nucleotides consisting of adenosine, guanosine, uridine, cytidine, and inosine.

292. The solid support according to any of claims 289 to 291, wherein the non-natural nucleobase of the one or more non-natural nucleotides is selected from the group consisting of 8-oxo-N⁶-methyladenine, 7-deazaxanthine, 7-deazaguanine, N⁴,N⁴-ethanocytosin, N⁶,N⁶-ethano-2,6-diamino-purine, 5-methylcytosine, 5-(C³—C⁶)-alkynylcytosine, 5-fluorouracil, 5-bromouracil, pseudoisocytosine, 2-hydroxy-5-methyl-4-triazolopyridine, isocytosine, isoguanine and inosine.

293. The solid support according to any of claims 289 to 292, wherein the non-natural backbone unit of the one or more non-natural nucleotides is selected from the group consisting of 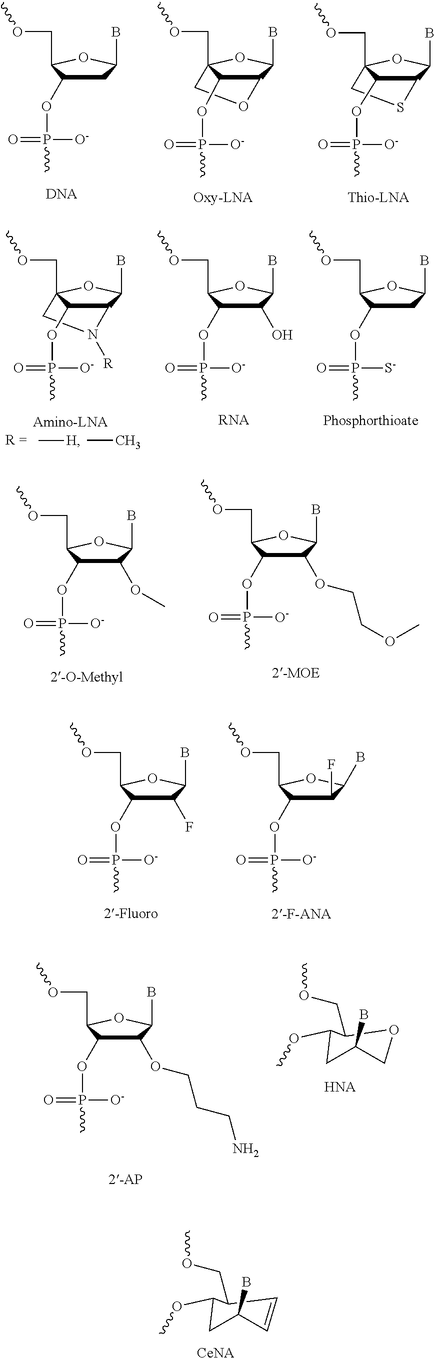 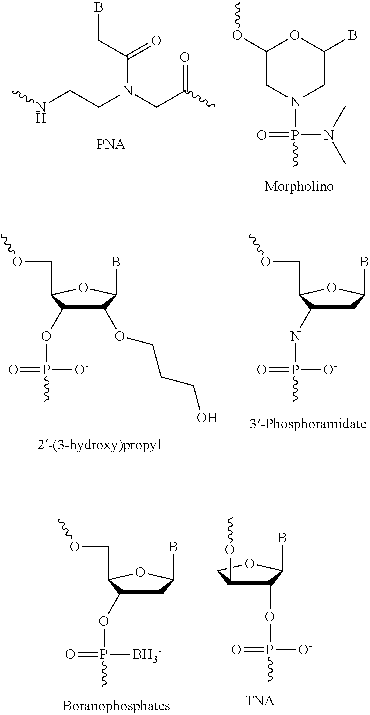
wherein B denotes a nucleobase.

294. The solid support according to any of claims 289 to 293, wherein the non-natural sugar moiety of the one or more non-natural backbone unit(s) is selected from the group consisting of 2′-deoxyribose, 2′-O-methyl-ribose, 2′-fluor-ribose and 2′-4′-O-methylene-ribose (LNA).

295. The solid support according to any of claims 289 to 294, wherein the non-natural internucleoside linker of the one or more non-natural backbone unit(s) is selected from the group consisting of phosphorothioate bonds, methylphosphonate bonds, phosphoramidate bonds, phosphotriester bonds and phosphodithioate bonds.

296. The solid support according to claim 269, wherein said 3′ overhang comprises naturally occurring nucleobases connected by naturally occurring backbone units, wherein said naturally occurring nucleobases and said naturally occurring backbone units do not prevent exonuclease degradation of said 3′ overhang.

297. The solid support according to claim 296, wherein said 3′ overhang further comprises non-naturally occurring nucleobases which do not prevent exonuclease degradation of said 3′ overhang.

298. The solid support according to claim 297, wherein said non-naturally occurring backbone units comprising sugar moieties and internucleoside linkers do not prevent exonuclease degradation of said 3′ overhang.

299. The solid support according to claim 298, wherein said sugar moieties are non-naturally occurring sugar moieties which do not prevent exonuclease degradation of said 3′ overhang.

300. The solid support according to claim 298, wherein said internucleoside linkers are non-naturally occurring internucleoside linkers which do not prevent exonuclease degradation of said 3′ overhang.

301. The solid support according to any of claims 296 to 300, wherein said one or more enzyme activities present in said sample comprises a 3′ to 5′ exonuclease activity capable of cleaving one or more, such as all of the internucleoside linkers connecting the nucleotides of the 3′ overhang and/or a ligase activity.

302. The solid support according to claim 301, wherein the circular oligonucleotide template capable of being amplified by rolling circle amplification is generated by ligating the oligonucleotide probe comprising a substrate moiety processed by 3′ to 5′ exonucleolytical digestion of the 3′ overhang, said ligation being performed by at least one ligase activity present in said sample.

303. The solid support according to claim 302, wherein said circular, oligonucleotide template is amplified by rolling circle amplification, said amplification being indicative of the presence in said sample of at least one 3′ to 5′ exonuclease activity and at least one ligase activity.

304. The solid support according to claim 303, wherein said rolling circle amplification product is detected by detecting a label covalently or non-covalently associated with said rolling circle amplification product, wherein said label is preferably fluorescently detectable, wherein said label is either a fluorescent molecule incorporated into the rolling circle amplification product, for example by being present in the primer used for probe amplification and generation of the rolling circle amplification product, or by being linked to a nucleotide incorporated into the rolling circle amplification product during the probe amplification process, or by being linked to a fluorescently labelled oligonucleotide hybridising to the rolling circle amplification product, or wherein said label is a molecule or a chemical group which can be detected by a fluorescently labelled molecule, such as an antibody.

305. The solid support according to claim 269, wherein said 3′ overhang comprises non-naturally occurring nucleobases connected by naturally occurring backbone units and/or non-naturally occurring backbone units, said backbone units comprising a sugar moiety and an internucleoside linker, wherein said non-naturally occurring nucleobases and said non-naturally occurring backbone units, when present, prevent exonuclease degradation of said 3′ overhang.

306. The solid support according to claim 305, wherein said non-naturally occurring nucleobases alone prevents exonuclease degradation of said 3′ overhang.

307. The solid support according to claim 305, wherein said non-naturally occurring backbone units prevent exonuclease degradation of said 3′ overhang.

308. The solid support according to claim 305, wherein said non-naturally occurring sugar moieties prevent exonuclease degradation of said 3′ overhang.

309. The solid support according to claim 305, wherein said non-naturally occurring internucleoside linkers prevent exonuclease degradation of said 3′ overhang.

310. The solid support according to any of claims 305 to 309, wherein a 3′ to 5′ exonuclease activity present in said sample cannot cleave the internucleoside linkers connecting the nucleotides of the 3′ overhang.

311. The solid support according to any of claims 305 to 309, wherein said one or more enzyme activities present in said sample further comprises a topoisomerase II activity capable of processing said unprocessed substrate moiety of said oligonucleotide probe.

312. The solid support according to claim 311, wherein a circular, oligonucleotide template capable of being amplified by rolling circle amplification is generated by said toposiomerase II activity.

313. The solid support according to claim 312, wherein said circular, oligonucleotide template is amplified by rolling circle amplification, said amplification being indicative of the presence in said sample of at least one topoisomerase II activity.

314. The solid support according to claim 313, wherein said rolling circle amplification product is detected by detecting a label covalently or non-covalently associated with said rolling circle amplification product, wherein said label is preferably fluorescently detectable, wherein said label is either a fluorescent molecule incorporated into the rolling circle amplification product, for example by being present in the primer used for probe amplification and generation of the rolling circle amplification product, or by being linked to a nucleotide incorporated into the rolling circle amplification product during the probe amplification process, or by being linked to a fluorescently labelled oligonucleotide hybridising to the rolling circle amplification product, or wherein said label is a molecule or a chemical group which can be detected by a fluorescently labelled molecule, such as an antibody.

315. A solid support comprising a plurality of attachment points for the attachment of one or more circular oligonucleotide templates to the solid support, wherein each attachment point is associated with one or more primers suitable for initiating rolling circle amplification of a circular oligonucleotide template generated by enzyme processing of an oligonucleotide probe comprising one or more unprocessed substrate moieties, said processing being performed according to the method of any of claims 1 to 155,
wherein the same or different primers are associated with the same or different attachment points, so that a plurality of circular oligonucleotide templates are attached to the solid support by means of hybridisation of each circular oligonucleotide template to said one or more primers associated with each of said plurality of attachment points,
wherein said circular oligonucleotide templates are selected from the group consisting of
i) circular oligonucleotide templates resulting from processing and ligation of oligonucleotide probes comprising unprocessed substrate moieties comprising or consisting of one or more nick(s) in one or more single strand(s) of a double stranded nucleotide sequence of said oligonucleotide probe, said one or more nick(s) forming one or more unprocessed substrate moieties of said oligonucleotide probe,
ii) circular oligonucleotide templates resulting from processing and ligation of oligonucleotide probes comprising unprocessed substrate moieties comprising or consisting of one or more single stranded nucleotide sequence(s) joined at one or both ends thereof by a double stranded nucleotide sequence, said single stranded sequence(s) creating one or more gap structure(s) forming one or more unprocessed substrate moieties of said oligonucleotide probe, and
iii) circular oligonucleotide templates resulting from processing and ligation of oligonucleotide probes comprising unprocessed substrate moieties comprising or consisting of one or more nick(s) or one or more gap(s), said gap(s) being in the form of a single stranded nucleotide sequence, said nick(s) or gap(s) being joined at one end thereof to a double stranded nucleotide sequence and at the other end thereof to at least one single stranded overhang joined to a double stranded nucleotide sequence of said oligonucleotide probe, wherein said nick(s) or gap(s) in combination with the at least one single stranded overhang forms one or more unprocessed substrate moieties of said oligonucleotide probe.

316. The solid support according to claim 315, wherein each primer attached to an attachment site at a different, predetermined position of the solid support comprises the same or a different label.

317. The solid support according to claim 316, wherein said different labels are selected from the group consisting of chromophores and fluorophores.

318. A microfluidic device comprising one or more reaction compartments for performing one or more rolling circle amplification events of a circular oligonucleotide template and one or more detection compartments for the detection of said rolling circle amplification events performed in said one or more reaction compartments.

319. The microfluidic device according to claim 318 further comprising the solid support according to any of claims 156 to 317.

320. A method for correlating one or more rolling circle amplification event(s) with the activity of one or more enzymes in a sample, said method comprising the steps of performing the method according to any of claims 1 to 155 and amplifying by rolling circle amplification the one or more circular templates having been generated as a result of the presence in said sample of said one or more enzyme activities, wherein the detection of said amplification events is done using the solid support according to any of claims 156 to 317 or the microfluidic device according to any of claims 318 and 319, wherein a predetermined number of rolling circle amplification events correlate with a predetermined enzyme activity, and wherein the actual number of rolling circle amplification events recorded for a given sample is compared to the number of events correlating with said predetermined enzyme activity, thereby correlating the actual number of rolling circle amplification events with said activity of said one or more enzyme activities present in said sample.

321. A method for testing the efficacy of a drug or drug-lead, said method comprising the steps of
i) providing a drug or drug-lead to be tested;
ii) providing a biological sample to be treated with the drug or drug-lead;
iii) performing the correlation method of claim 320 for the biological sample in the absence of drug or drug-lead and determining the activity of one or more enzyme activities involved in circularising a non-circular oligonucleotide probe;
iv) contacting the drug or drug-lead and the biological sample;
v) performing the correlation method of claim 320 for the biological sample in the presence of drug or drug-lead and determining the activity of one or more enzyme activities involved in circularising a non-circular oligonucleotide probe;
vi) comparing the enzyme activities in the biological sample in the presence and absence, respectively, of the drug or drug-lead, wherein said comparison is obtained by comparing the rolling circle amplification events in the presence and absence, respectively, of the drug or drug-lead, and
vii) evaluating the efficacy of the drug or drug-lead based on the comparison performed in step vi).

322. A method for diagnosing or prognosing a disease in an individual by determining the activity of one or more enzyme activities involved in circularising a non-circular oligonucleotide probe, said method comprising the steps of
i) obtaining a biological sample from an individual to be tested, said biological sample comprising said one or more enzyme activities to be tested in the diagnostic or prognostic method,
ii) performing on said biological sample the method according to any of claims 1 to 155 and amplifying by rolling circle amplification the one or more circular templates having been generated as a result of the presence in said sample of said one or more enzyme activities being tested for, and optionally detecting said amplification events by using the solid support according to any of claims 156 to 317 or the microfluidic device according to any of claims 318 and 319, and
iii) determining the number of rolling circle amplification events and
iv) correlating said number of rolling amplification events with a predetermined enzyme activity corresponding to standard defining a physiologically normal activity of the one or more enzyme activities being tested for in a healthy individual,
wherein the actual number of rolling circle amplification events recorded for a given sample is compared to the number of events correlating with said predetermined enzyme activity, thereby correlating the actual number of rolling circle amplification events with said activity of said one or more enzyme activities present in said sample, and diagnosing or prognosing said individual with said disease, or the likelihood of developing said disease, based on the enzyme activities determined in said biological sample.

323. A method for treating a disease diagnosed according to the method of claim 322, said method comprising the steps of administering a pharmaceutical composition to said individual having being diagnosed with said disease, wherein said medicament is capable of treating said disease by curing the disease or ameliorating the disease.

324. A method for treating prophylactically a disease prognosed according to the method of claim 322, said method comprising the steps of administering a pharmaceutical composition to said individual having being prognosed with the likelihood of developing said disease, wherein said pharmaceutical composition is capable of treating prophylactically said disease.

325. The method of any of claims 322 to 324, wherein said disease is cancer.

326. The method of claim 325, wherein said cancer disease is selected from the group consisting of bladder carcinoma, blood (and bone marrow)-hematological malignancies, leukemia, lymphoma, Hodgkin's disease, non-Hodgkin's lymphoma, multiple myeloma, brain tumor, breast cancer, cervical cancer, colorectal cancer—in the colon, rectum, anus, or appendix, esophageal cancer, endometrial cancer—in the uterus, hepatocellular carcinoma—in the liver, gastrointestinal stromal tumor (GIST), laryngeal cancer, lung cancer, mesothelioma—in the pleura or pericardium, oral cancer, osteosarcoma—in bones, ovarian cancer, pancreatic cancer, prostate cancer, renal cell carcinoma—in the kidneys, rhabdomyosarcoma—in muscles, skin cancer (including benign moles and dysplastic nevi), stomach cancer, testicular cancer, and thyroid cancer.

327. The method of claim 325, wherein said cancer disease is selected from the group consisting of neuroblastoma, leukemia, a cancer in the central nervous system, retinoblastoma, Wilms' tumor, germ cell cancer, soft tissue sarcomas, hepatic cancer, lymphomas, and epithelial cancer.

328. The method of any of claims 322 to 324, wherein said disease is related to cellular aging.

329. The method of claim 328, wherein the disease related to cellular aging is selected from the group consisting of Alzheimer's Disease, Creutzfeld-Jakob Disease, Dementia, Multiple Systems Atrophy, Neurodegenerative Diseases, such as Parkinsonism, Retrogenesis, Sundown Syndrome and Vascular Dementia.
